(19) United States
(12) Patent Application Publication
(10) Pub. No.: US 2026/0234213 A1
(43) Pub. Date: Aug. 13, 2026

(54) RELAXIN-2 FUSION PROTEIN ANALOGS AND METHODS OF USING SAME

(71) Applicant: TECTONIC OPERATING COMPANY, INC., Watertown, MA (US)

(72) Inventors: John DIENER, Watertown, MA (US); Andrew KRUSE, Watertown, MA (US); Franz GRUSWITZ, Watertown, MA (US)

(21) Appl. No.: 19/397,507

(22) Filed: Nov. 21, 2025

Related U.S. Application Data
(62) Division of application No. 18/054,596, filed on Nov. 11, 2022, now Pat. No. 12,509,497.
(60) Provisional application No. 63/263,917, filed on Nov. 11, 2021.

(51) Int. Cl. C07K 14/64; A61P 43/00; C12N 15/63

(52) U.S. Cl. CPC C07K 14/64; A61P 43/00; C12N 15/63; C07K 2319/30

(57) ABSTRACT

The present disclosure provides relaxin-2 fusion protein analogs with enhanced in vivo half-life and methods for making the same. Also disclosed herein are methods of treating relaxin-2-associated disorders or diseases using the relaxin-2 fusion protein analogs described herein.

### RELATED APPLICATIONS

This application is a continuation of U.S. patent application Ser. No. 18/054,596, filed Nov. 11, 2022, which claims priority to U.S. Provisional Patent Application Ser. No. 63/263,917, filed Nov. 11, 2021, the entire disclosures of which are hereby incorporated herein by reference.

### REFERENCE TO SEQUENCE LISTING

This application contains a sequence listing which has been submitted electronically in ASCII format and is hereby incorporated by reference in its entirety (said ASCII copy, created Nov. 11, 2022, is named “TECW-002_SL_ST26_2022-11-11” and is 284,217 bytes in size).

### FIELD

This disclosure provides relaxin-2 fusion protein analogs with improved pharmacokinetic properties, methods for making these fusion proteins, and methods of using these fusion proteins to enhance relaxin-2 related activity in a subject and treat or prevent relaxin-2 related diseases.

### BACKGROUND

Relaxin-2 exhibits strong antifibrotic activity. In injured tissues, fibroblast activation and proliferation cause increased collagen production and interstitial fibrosis. Fibrosis in the heart is increased by biomechanical overload, and influences ventricular dysfunction, remodeling, and arrhythmogenesis. However, due to the limited in vivo half-life of relaxin, treatment of patients has to be repeated every 14 to 21 days, whereby compound administration has to be performed as a continuous infusion for at least 48 hours. Further, the synthesis of relaxin-2 is difficult. Due to the low solubility of the B-chain and the requirement for the laborious, specific introduction of cysteine bridges between A and B-chains, yields of active peptide obtained by these methods are extremely low.

There is a need for an engineered relaxin-2 analog with greater half-life and greater ease in production.

### SUMMARY

This disclosure provides fusion proteins that are engineered relaxin-2 analogs with improved pharmacokinetic properties. This disclosure also provides methods of using these fusion proteins to enhance relaxin-2 related activity in a subject and to treat or prevent relaxin-2 related diseases.

Provided herein is a fusion protein comprising, from N-terminus to C-terminus, a first peptide; a linker peptide; and a second peptide, wherein the first peptide comprises an amino acid sequence that differs at 0, 1, 2, 3, 4, or 5 amino acids when compared to the amino acid sequence of SEQ ID NO: 7 and the second peptide comprises an amino acid sequence that differs at 0, 1, 2, 3, 4, or 5 amino acids when compared to the amino acid sequence of SEQ ID NO: 9; or the first peptide comprises an amino acid sequence that differs at 0, 1, 2, 3, 4, or 5 amino acids when compared to the amino acid sequence of SEQ ID NO: 9 and the second peptide comprises an amino acid sequence that differs at 0, 1, 2, 3, 4, or 5 amino acids when compared to the amino acid sequence of SEQ ID NO: 7; the linker peptide comprises an amino acid sequence with 12-15 amino acids, comprising 2-5 acidic amino acids and 10-13 non-acidic amino acids; and the fusion protein has a pI from 6.0 to 8.2.

In some embodiments, the linker peptide comprises an amino acid sequence selected from the group consisting of

R₁R₁R₁R₂R₁R₁R₁R₂R₁R₁R₁R₂R₁;
R₁R₁R₁R₂R₁R₁R₁R₂R₁R₁R₁R₂R₁R₁R₁;
R₁R₁R₂R₁R₁R₁R₂R₂R₁R₁R₁R₂R₁R₁;
R₁R₁R₁R₂R₂R₁R₁R₁R₂R₂R₁R₁R₁; and
R₁R₁R₂R₁R₂R₁R₁R₂R₁R₂R₁R₁R₁,

wherein R₁ is a non-acidic amino acid and R₂ is an acidic amino acid.

In some embodiments, the acidic amino acid(s) are aspartate or glutamate. In some embodiments, the acidic amino acid(s) are glutamate. In some embodiments, the non-acidic amino acid(s) are glycine, proline, or serine. In some embodiments, the non-acidic amino acid(s) are glycine.

In some embodiments, the linker peptide comprises the amino acid sequence of one or more of SEQ ID NO: 14, 15, 16, 17, or 18. In some embodiments, the linker peptide comprises an amino acid sequence selected from the group consisting of SEQ ID NOs: 19-23.

In some embodiments, the first peptide comprises the amino acid sequence of DSWX₃EEVIKLCGRELVRAQIAICGX₄ST (SEQ ID NO: 3), wherein X₃ is methionine, lysine, or glutamine, and wherein X₄ is methionine or lysine. In some embodiments, the first peptide comprises the amino acid sequence of X₅QX₆YSALANKCCHVGCTKRSLAX₇FC (SEQ ID NO: 4), wherein X₅ is arginine or absent, X₆ is leucine or aspartic acid, and wherein X₇ is arginine, glutamine, or glutamate.

In some embodiments, the second peptide comprises the amino acid sequence of DSWX₃EEVIKLCGRELVRAQIAICGX₄ST (SEQ ID NO: 3), wherein X₃ is methionine, lysine, or glutamine, and wherein X₄ is methionine or lysine. In some embodiments, the second peptide comprises the amino acid sequence of X₅QX₆YSALANKCCHVGCTKRSLAX₇FC (SEQ ID NO: 4), wherein X₅ is arginine or absent, X₆ is leucine or aspartate, and wherein X₇ is arginine, glutamine or glutamate.

In some embodiments, the first peptide comprises an amino acid sequence selected from the group consisting of SEQ ID NOs: 5-7. In some embodiments, the second peptide comprises an amino acid sequence selected from the group consisting of SEQ ID NOs: 5-7. In some embodiments, the first peptide comprises an amino acid sequence selected from the group consisting of SEQ ID NOs: 8-13. In some embodiments, the second peptide comprises an amino acid sequence selected from the group consisting of SEQ ID NOs: 8-13.

In some embodiments, the first peptide comprises the amino acid sequence of SEQ ID NO: 5 and the second peptide comprises the amino acid sequence of SEQ ID NO: 8; the first peptide comprises the amino acid sequence of SEQ ID NO: 5 and the second peptide comprises the amino acid sequence of SEQ ID NO: 9; the first peptide comprises the amino acid sequence of SEQ ID NO: 5 and the second peptide comprises the amino acid sequence of SEQ ID NO: 10; the first peptide comprises the amino acid sequence of SEQ ID NO: 5 and the second peptide comprises the amino acid sequence of SEQ ID NO: 11; the first peptide comprises the amino acid sequence of SEQ ID NO: 5 and the second peptide comprises the amino acid sequence of SEQ ID NO: 12; the first peptide comprises the amino acid sequence of SEQ ID NO: 5 and the second peptide comprises the amino acid sequence of SEQ ID NO: 13; the first peptide comprises the amino acid sequence of SEQ ID NO: 6 and the second peptide comprises the amino acid sequence of SEQ ID NO: 8; the first peptide comprises the amino acid sequence of SEQ ID NO: 6 and the second peptide comprises the amino acid sequence of SEQ ID NO: 9; the first peptide comprises the amino acid sequence of SEQ ID NO: 6 and the second peptide comprises the amino acid sequence of SEQ ID NO: 10; the first peptide comprises the amino acid sequence of SEQ ID NO: 6 and the second peptide comprises the amino acid sequence of SEQ ID NO: 11; the first peptide comprises the amino acid sequence of SEQ ID NO: 6 and the second peptide comprises the amino acid sequence of SEQ ID NO: 12; the first peptide comprises the amino acid sequence of SEQ ID NO: 6 and the second peptide comprises the amino acid sequence of SEQ ID NO: 13; the first peptide comprises the amino acid sequence of SEQ ID NO: 7 and the second peptide comprises the amino acid sequence of SEQ ID NO: 8; the first peptide comprises the amino acid sequence of SEQ ID NO: 7 and the second peptide comprises the amino acid sequence of SEQ ID NO: 9; the first peptide comprises the amino acid sequence of SEQ ID NO: 7 and the second peptide comprises the amino acid sequence of SEQ ID NO: 10; the first peptide comprises the amino acid sequence of SEQ ID NO: 7 and the second peptide comprises the amino acid sequence of SEQ ID NO: 11; the first peptide comprises the amino acid sequence of SEQ ID NO: 7 and the second peptide comprises the amino acid sequence of SEQ ID NO: 12; or the first peptide comprises the amino acid sequence of SEQ ID NO: 7 and the second peptide comprises the amino acid sequence of SEQ ID NO: 13.

In some embodiments, the second peptide comprises the amino acid sequence of SEQ ID NO: 5 and the first peptide comprises the amino acid sequence of SEQ ID NO: 8; the second peptide comprises the amino acid sequence of SEQ ID NO: 5 and the first peptide comprises the amino acid sequence of SEQ ID NO: 9; the second peptide comprises the amino acid sequence of SEQ ID NO: 5 and the first peptide comprises the amino acid sequence of SEQ ID NO: 10; the second peptide comprises the amino acid sequence of SEQ ID NO: 5 and the first peptide comprises the amino acid sequence of SEQ ID NO: 11; the second peptide comprises the amino acid sequence of SEQ ID NO: 5 and the first peptide comprises the amino acid sequence of SEQ ID NO: 12; the second peptide comprises the amino acid sequence of SEQ ID NO: 5 and the first peptide comprises the amino acid sequence of SEQ ID NO: 13; the second peptide comprises the amino acid sequence of SEQ ID NO: 6 and the first peptide comprises the amino acid sequence of SEQ ID NO: 8; the second peptide comprises the amino acid sequence of SEQ ID NO: 6 and the first peptide comprises the amino acid sequence of SEQ ID NO: 9; the second peptide comprises the amino acid sequence of SEQ ID NO: 6 and the first peptide comprises the amino acid sequence of SEQ ID NO: 10; the second peptide comprises the amino acid sequence of SEQ ID NO: 6 and the first peptide comprises the amino acid sequence of SEQ ID NO: 11; the second peptide comprises the amino acid sequence of SEQ ID NO: 6 and the first peptide comprises the amino acid sequence of SEQ ID NO: 12; the second peptide comprises the amino acid sequence of SEQ ID NO: 6 and the first peptide comprises the amino acid sequence of SEQ ID NO: 13; the second peptide comprises the amino acid sequence of SEQ ID NO: 7 and the first peptide comprises the amino acid sequence of SEQ ID NO: 8; the second peptide comprises the amino acid sequence of SEQ ID NO: 7 and the first peptide comprises the amino acid sequence of SEQ ID NO: 9; the second peptide comprises the amino acid sequence of SEQ ID NO: 7 and the first peptide comprises the amino acid sequence of SEQ ID NO: 10; the second peptide comprises the amino acid sequence of SEQ ID NO: 7 and the first peptide comprises the amino acid sequence of SEQ ID NO: 11; the second peptide comprises the amino acid sequence of SEQ ID NO: 7 and the first peptide comprises the amino acid sequence of SEQ ID NO: 12; or the second peptide comprises the amino acid sequence of SEQ ID NO: 7 and the first peptide comprises the amino acid sequence of SEQ ID NO: 13.

In some embodiments, the fusion protein comprises an amino acid sequence selected from the group consisting of SEQ ID NOs: 25-48.

In some embodiments, the first peptide consists of an amino acid sequence selected from the group consisting of SEQ ID NOs: 5-7. In some embodiments, the second peptide consists of an amino acid sequence selected from the group consisting of SEQ ID NOs: 5-7. In some embodiments, the first peptide consists of an amino acid sequence selected from the group consisting of SEQ ID NOs: 8-13. In some embodiments, the second peptide consists of an amino acid sequence selected from the group consisting of SEQ ID NOs: 8-13.

In some embodiments, the first peptide consists of the amino acid sequence of SEQ ID NO: 5 and the second peptide consists of the amino acid sequence of SEQ ID NO: 8; the first peptide consists of the amino acid sequence of SEQ ID NO: 5 and the second peptide consists of the amino acid sequence of SEQ ID NO: 9; the first peptide consists of the amino acid sequence of SEQ ID NO: 5 and the second peptide consists of the amino acid sequence of SEQ ID NO: 10; the first peptide consists of the amino acid sequence of SEQ ID NO: 5 and the second peptide consists of the amino acid sequence of SEQ ID NO: 11; the first peptide consists of the amino acid sequence of SEQ ID NO: 5 and the second peptide consists of the amino acid sequence of SEQ ID NO: 12; the first peptide consists of the amino acid sequence of SEQ ID NO: 5 and the second peptide consists of the amino acid sequence of SEQ ID NO: 13; the first peptide consists of the amino acid sequence of SEQ ID NO: 6 and the second peptide consists of the amino acid sequence of SEQ ID NO: 8; the first peptide consists of the amino acid sequence of SEQ ID NO: 6 and the second peptide consists of the amino acid sequence of SEQ ID NO: 9; the first peptide consists of the amino acid sequence of SEQ ID NO: 6 and the second peptide consists of the amino acid sequence of SEQ ID NO: 10; the first peptide consists of the amino acid sequence of SEQ ID NO: 6 and the second peptide consists of the amino acid sequence of SEQ ID NO: 11; the first peptide consists of the amino acid sequence of SEQ ID NO: 6 and the second peptide consists of the amino acid sequence of SEQ ID NO: 12; the first peptide consists of the amino acid sequence of SEQ ID NO: 6 and the second peptide consists of the amino acid sequence of SEQ ID NO: 13; the first peptide consists of the amino acid sequence of SEQ ID NO: 7 and the second peptide consists of the amino acid sequence of SEQ ID NO: 8; the first peptide consists of the amino acid sequence of SEQ ID NO: 7 and the second peptide consists of the amino acid sequence of SEQ ID NO: 9; the first peptide consists of the amino acid sequence of SEQ ID NO: 7 and the second peptide consists of the amino acid sequence of SEQ ID NO: 10; the first peptide consists of the amino acid sequence of SEQ ID NO: 7 and the second peptide consists of the amino acid sequence of SEQ ID NO: 11; the first peptide consists of the amino acid sequence of SEQ ID NO: 7 and the second peptide consists of the amino acid sequence of SEQ ID NO: 12; or the first peptide consists of the amino acid sequence of SEQ ID NO: 7 and the second peptide consists of the amino acid sequence of SEQ ID NO: 13.

In some embodiments, the second peptide consists of the amino acid sequence of SEQ ID NO: 5 and the first peptide consists of the amino acid sequence of SEQ ID NO: 8; the second peptide consists of the amino acid sequence of SEQ ID NO: 5 and the first peptide consists of the amino acid sequence of SEQ ID NO: 9; the second peptide consists of the amino acid sequence of SEQ ID NO: 5 and the first peptide consists of the amino acid sequence of SEQ ID NO: 10; the second peptide consists of the amino acid sequence of SEQ ID NO: 5 and the first peptide consists of the amino acid sequence of SEQ ID NO: 11; the second peptide consists of the amino acid sequence of SEQ ID NO: 5 and the first peptide consists of the amino acid sequence of SEQ ID NO: 12; the second peptide consists of the amino acid sequence of SEQ ID NO: 5 and the first peptide consists of the amino acid sequence of SEQ ID NO: 13; the second peptide consists of the amino acid sequence of SEQ ID NO: 6 and the first peptide consists of the amino acid sequence of SEQ ID NO: 8; the second peptide consists of the amino acid sequence of SEQ ID NO: 6 and the first peptide consists of the amino acid sequence of SEQ ID NO: 9; the second peptide consists of the amino acid sequence of SEQ ID NO: 6 and the first peptide consists of the amino acid sequence of SEQ ID NO: 10; the second peptide consists of the amino acid sequence of SEQ ID NO: 6 and the first peptide consists of the amino acid sequence of SEQ ID NO: 11; the second peptide consists of the amino acid sequence of SEQ ID NO: 6 and the first peptide consists of the amino acid sequence of SEQ ID NO: 12; the second peptide consists of the amino acid sequence of SEQ ID NO: 6 and the first peptide consists of the amino acid sequence of SEQ ID NO: 13; the second peptide consists of the amino acid sequence of SEQ ID NO: 7 and the first peptide consists of the amino acid sequence of SEQ ID NO: 8; the second peptide consists of the amino acid sequence of SEQ ID NO: 7 and the first peptide consists of the amino acid sequence of SEQ ID NO: 9; the second peptide consists of the amino acid sequence of SEQ ID NO: 7 and the first peptide consists of the amino acid sequence of SEQ ID NO: 10; the second peptide consists of the amino acid sequence of SEQ ID NO: 7 and the first peptide consists of the amino acid sequence of SEQ ID NO: 11; the second peptide consists of the amino acid sequence of SEQ ID NO: 7 and the first peptide consists of the amino acid sequence of SEQ ID NO: 12; or the second peptide consists of the amino acid sequence of SEQ ID NO: 7 and the first peptide consists of the amino acid sequence of SEQ ID NO: 13.

In some embodiments, the fusion protein further comprises an IgG Fc. In some embodiments, the IgG Fc comprises the amino acid alanine at EU positions 234 and 235. In some embodiments, the IgG Fc comprises the amino acid alanine at EU position 329. In some embodiments, the IgG Fc comprises the amino acid alanine at EU positions 234, 235 and 329. In some embodiments, the IgG Fc comprises the amino acids alanine, alanine, alanine, leucine, and serine at EU positions 234, 235, 329, 428 and 434, respectively. In some embodiments, the IgG Fc comprises the amino acids lysine, phenylalanine, and tyrosine at EU positions 433, 434 and 436, respectively. In some embodiments, the IgG Fc comprises the amino acids tyrosine, threonine and glutamate at EU positions 252, 254 and 256, respectively. In some embodiments, the IgG Fc comprises the amino acids leucine and serine at EU positions 428 and 434, respectively.

In some embodiments, the IgG Fc comprises an amino acid sequence at least 85% identical to the amino acid sequence of a human IgG1 Fc. In some embodiments, the IgG Fc comprises the amino acid sequence of a human IgG1 Fc. In some embodiments, the IgG Fc comprises an amino acid sequence at least 95% identical to an amino acid sequence selected from the group consisting of SEQ ID NOs: 50-52 and 201-203. In some embodiments, the IgG Fc comprises an amino acid sequence selected from the group consisting of SEQ ID NOs: 50-52 and 201-203.

In some embodiments, the IgG Fc is linked to the N-terminus of the first peptide. In some embodiments, the IgG Fc is linked to the C-terminus of the second peptide.

In some embodiments, the fusion protein comprises an amino acid sequence selected from the group consisting of SEQ ID NOs: 53-85 and 204-211. In some embodiments, the fusion protein comprises an amino acid sequence selected from the group consisting of SEQ ID NOs: 86-118 and 214-221.

In some embodiments, the fusion protein consists of an amino acid sequence selected from the group consisting of SEQ ID NOs: 53-85 and 204-211. In some embodiments, the fusion protein consists of an amino acid sequence selected from the group consisting of SEQ ID NOs: 86-118 and 214-221.

In some embodiments, the first and second peptides do not comprise the amino acid sequence of a peptide selected from the group consisting of

(SEQ ID NO: 187)
DSWKEEVIKLCGRELVRAQIAICGKSTAS;
(SEQ ID NO: 188)
DSWKEEVIKLCGRELVRAQIAICGKSTWS;
(SEQ ID NO: 189)
DSWMEEVIKLCGRELVRAQIAICGKSTAS;
and
(SEQ ID NO: 190)
DSWMEEVIKLCGRELVRAQIAICGKSTWS.

Also provided herein is a fusion protein comprising an amino acid sequence selected from the group consisting of SEQ ID NOs: 53-118 and 204-225. In some embodiments, the fusion protein consists of an amino acid sequence selected from the group consisting of SEQ ID NOs: 53-118 and 204-225.

Also provided herein is a polynucleotide comprising a nucleotide sequence encoding any of the fusion proteins provided above and herein. In some embodiments, the polynucleotide is an RNA molecule. In some embodiments, the polynucleotide is a DNA molecule.

Also provided herein is an expression vector comprising any polynucleotides described above and herein. In some embodiments, the expression vector is a plasmid. In some embodiments, the expression vector is a viral vector.

Also provided herein is a host cell comprising any polynucleotides or expression vectors described above and herein. In some embodiments, the host cell is a prokaryotic cell. In some embodiments, the host cell is a eukaryotic cell. In some embodiments, the prokaryotic cell an E. coli cell or a Bacillus cell. In some embodiments, the eukaryotic cell is selected from the group consisting of a yeast cell, an insect cell, and a mammalian cell. In some embodiments, the mammalian cell is selected from the group consisting of a CHO cell, a HeLa cell, and a 293 cell.

Also provided herein is a population of cells comprising two or more of any of the host cells described above and herein.

Also provided herein is a method of producing any of the fusion proteins described above and herein comprising culturing any of the host cells described above and herein under conditions such that the fusion protein is produced.

Also provided herein is a pharmaceutical composition comprising an effective amount of any of the fusion proteins described above and herein, or any polynucleotides described above and herein, or any expression vectors described above and herein.

Also provided herein is a method of enhancing a relaxin-2-related activity in a primary cell, comprising contacting the primary cell with any of the fusion proteins described above and herein, thereby enhancing relaxin-2-related activity in the cell. In some embodiments, the fusion protein activates the relaxin-2 receptor, RXFP1, on a cell surface. In some embodiments, the method elevates cAMP levels in the primary cell, inducing vasodilation, inducing the expression of angiogenic factors, inducing the expression of MMPs, and inducing collagen degradation. In some embodiments, the primary cell is selected from the group consisting of endothelial cells, vascular smooth muscle cells, other vascular cells, cardiomyocytes, other cardiac cells, and fibroblasts. In some embodiments, the primary cell is within a subject. In some embodiments, the subject has a relaxin-2-associated disorder. In some embodiments, the relaxin-2-associated disorder is selected from the group consisting of kidney diseases, fibrotic diseases, and cardiovascular diseases. In some embodiments, the disorder is selected from the group consisting of focal segmental glomerular sclerosis (FSGS), diabetic nephropathy, hepatorenal syndrome, scleroderma, idiopathic pulmonary fibrosis, renal fibrosis, cardiac fibrosis, NASH, dilated cardiomyopathy, diastolic heart failure, pulmonary arterial hypertension, chronic heart failure, acute heart failure, congestive heart failure, coronary artery disease, hypertension, and pre-eclampsia.

Also provided herein is a method of treating a relaxin-associated disorder in a subject in need thereof, comprising administering to the subject an effective amount of any of the fusion proteins described above and herein, any polynucleotides described above and herein, any expression vectors described above and herein, or any pharmaceutical composition described above and herein, thereby treating the relaxin-associated disorder. In some embodiments, the relaxin-2-associated disorder is selected from the group consisting of kidney diseases, fibrotic diseases, and cardiovascular diseases. In some embodiments, the disorder is selected from the group consisting of focal segmental glomerular sclerosis (FSGS), diabetic nephropathy, hepatorenal syndrome, scleroderma, idiopathic pulmonary fibrosis, renal fibrosis, cardiac fibrosis, NASH, dilated cardiomyopathy, diastolic heart failure, pulmonary arterial hypertension, chronic heart failure, acute heart failure, congestive heart failure, coronary artery disease, hypertension, and pre-eclampsia. In some embodiments, the method decreases arterial pressure, increases renal artery blood flow, increases cardiac filling at diastole, resolves established fibrosis, or suppresses new fibrosis development.

Also provided herein is a kit comprising an effective amount any of the fusion proteins described above and herein, any polynucleotides described above and herein, any expression vectors described above and herein, or any pharmaceutical composition described above and herein, and an instruction of use.

### DETAILED DESCRIPTION

The disclosure provides fusion proteins comprising a human relaxin-2 B chain, or a derivative thereof, and a human relaxin-2 A chain, or a derivative thereof, joined by a peptide linker, wherein the fusion proteins have high in vivo circulating half-life when administered to mammals. In some embodiments, the in vivo circulating half-life of the fusion proteins provided in this disclosure is greater than 2 hours. In some embodiments, the fusion proteins provided in this disclosure have low pI. In some embodiments, the pI of the fusion proteins provided in this disclosure is less than 8.5. In some embodiments, the low pI of the fusion proteins provided in this disclosure is caused by acidic amino acid residues present in the peptide linker. In some embodiments, the peptide linker of the fusion protein comprises 2 or more acidic amino acids. In some embodiments, the peptide linker is 10-15 total amino acids in length.

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include,” “includes,” and “included,” is not limiting. The section headings used herein are for organizational purposes only and are not to be construed as limiting the subject matter described.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “protein” and “polypeptide” are used interchangeably herein and refer to a polymer of amino acids connected by one or more peptide bonds. As used herein, “amino acid sequence” refers to the information describing the relative order and identity of amino acid residues which make up a polypeptide.

As used herein, the term “an amino acid sequence that differs at 1 or more amino acids,” with reference to an amino acid sequence, refers to an amino acid sequence that comprises at least one substitution, alteration, inversion, addition, or deletion of an amino acid residue compared to a reference amino acid sequence.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F, (1990) PNAS 87: 2264-2268, modified as in Karlin S & Altschul S F, (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., at score=100, wordlength=12 to obtain nucleotide sequences homologous to a nucleic acid molecule described herein. BLAST protein searches can be performed with the XBLAST program parameters set, e.g., at score=50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules. Id. When utilizing BLAST, Gapped BLAST, and PSI BLAST programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, (1988) CABIOS 4:11-17, which is herein incorporated by reference in its entirety. Such an algorithm is incorporated in the ALIGN program (version 2.0) which is part of the GCG sequence alignment software package.

When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety.

As used herein, the terms “human relaxin-2 B chain” or “relaxin B chain” or “relaxin B” or “rel B” refer to a peptide comprising or consisting of the amino acid sequence as set forth in DSWMEEVIKLCGRELVRAQIAICGMSTWS (SEQ ID NO: 1) or derivatives thereof. In some embodiments, a derivative of a relaxin B chain comprises the amino acid sequence of SEQ ID NO: 1 with 1, 2, 3, 4, or 5 amino acid changes.

As used herein, the terms “human relaxin-2 A chain” or “relaxin A chain” or “relaxin A” or “rel A” refer to a peptide comprising or consisting of the amino acid sequence as set forth in QLYSALANKCCHVGCTKRSLARFC (SEQ ID NO: 2) or derivatives thereof. In some embodiments, a derivative of a relaxin A chain comprises the amino acid sequence of SEQ ID NO: 2 with 1, 2, 3, 4, or 5 amino acid changes.

As used herein, the term “linker peptide” refers to a peptide that links the relaxin A chain and the relaxin B chain in the fusion proteins described herein.

As used herein, the term “acidic amino acid” refers to an amino acid that has a carboxylic acid in its side chain. In some embodiments, the acidic amino acid is aspartate, glutamate, 2-aminoadipic acid, 2-aminobutyric acid or 2-aminopimelic acid. In some embodiments, acid amino acids include aspartate and glutamate.

As used herein, the term “non-acidic amino acid” refers to amino acids that are not acidic amino acids. In some embodiments, non-acidic amino acids include glycine, proline, and serine. In some embodiments, non-specific amino acids also include arginine, histidine, lysine, threonine, asparagine, glutamine, cysteine, alanine, valine, isoleucine, leucine, methionine, phenylalanine, tyrosine, and tryptophan.

As used herein, the term “IgG Fc” refers to the immunoglobulin G (IgG) fragment crystallizable (Fc) region. In some embodiments, the IgG Fc is the human IgG1, IgG2, IgG3, or IgG4 Fc region. In some embodiments, the IgG Fc is the IgG1 Fc region.

As used herein, the term “EU numbering system” refers to the EU numbering convention for the constant regions of an antibody, as described in Edelman, G. M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety.

As used herein, the term “relaxin-2 receptor,” “human relaxin-2 receptor,” “human relaxin receptor 1,” “RXFP1,” or “LGR7” is the native receptor of relaxin-2 in humans. In some embodiments, RXFP1 comprises the amino acid sequence shown in NCBI Reference Sequence: NP_067647.2, NP_001240656.1, NP_001240657.1, NP_001240658.1, NP_001240659.1, NP_001240661.1, NP_001240662.1, or NP_001350705.1 incorporated herein by reference in its entirety.

As used herein, the terms “treat,” “treating,” and “treatment” refer to therapeutic or preventative measures described herein. In some embodiments, the methods of “treatment” employ administration of a fusion protein to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

As used herein, the term “pI” means the isoelectric point, i.e., the pH of a solution at which the next charge on a fusion protein is zero. In some embodiments, the pI is the calculated or theoretical pI. In some embodiments, the pI is measured experimentally by an instrument.

### Fusion Proteins

The disclosure provides fusion proteins comprising a human relaxin-2 B chain, or a derivative thereof, and a human relaxin-2 A chain, or a derivative thereof, linked by a peptide linker, wherein the fusion proteins have high in vivo circulating half-life when administered to mammals. In some embodiments, the fusion protein comprises, from N-terminus to C-terminus, a human relaxin-2 B chain, or a derivative thereof, a peptide linker and a human relaxin-2 A chain, or a derivative thereof. In some embodiments, the fusion protein comprises, from N-terminus to C-terminus, a human relaxin-2 A chain, or a derivative thereof, a peptide linker and a human relaxin-2 B chain, or a derivative thereof. In some embodiments, the fusion protein further comprises an IgG Fc. The IgG Fc is linked to the N-terminus or C-terminus of the human relaxin B chain-linker protein-human relaxin A chain fusion protein or the human relaxin A chain-linker protein-human relaxin B chain fusion protein. In some embodiments, the IgG Fc described above is replaced with PEG.

### Human Relaxin-2 B Chain Derivatives

The disclosure provides human relaxin-2 B chain derivatives, wherein the derivatives have 1, 2, 3, 4, or 5 amino acid changes when compared to the amino acid sequence of SEQ ID NO: 1. In some embodiments, the amino acid that corresponds with position 13 of SEQ ID NO: 1 must be arginine. In some embodiments, the amino acid that corresponds with position 17 of SEQ ID NO: 1 must be arginine. In some embodiments, the amino acid that corresponds with position 20 of SEQ ID NO: 1 must be isoleucine. In some embodiments, the amino acid that corresponds with position 13 of SEQ ID NO: 1 must be arginine; the amino acid that corresponds with position 17 of SEQ ID NO: 1 must be arginine; and the amino acid that corresponds with position 20 of SEQ ID NO: 1 must be isoleucine.

In some embodiments, the human relaxin-2 B chain derivatives comprise or consist of the following formula: DSWX₃EEVIKLCGRELVRAQIAICGX₄ST (SEQ ID NO: 3), wherein X₃ and X₄ are absent or any amino acid. In some embodiments, X₃ is methionine, lysine or glutamine, and X₄ is methionine or lysine. In some embodiments, X₄ is lysine.

In some embodiments, the human relaxin-2 B chain derivatives used in the fusion proteins described herein do not include the amino acid sequences of SEQ ID NOs: 187-190.

In some embodiments, the human relaxin-2 B chain derivatives are from 22, 23, 24, 25, 26, 27, 28, 29, 30, 31, or 32 amino acids in length. In some embodiments, the human relaxin-2 B chain derivatives are 25, 26, 27, 28, or 29 amino acids in length. In some embodiments, the human relaxin-2 B chain derivatives are 27 amino acids in length.

In some embodiments, the human relaxin-2 B chain derivatives comprise or consist of the amino acid sequences shown in Table 1, below.

TABLE 1
Human Relaxin-2 B Chain Derivative Sequences

| SEQ ID NO: | Amino Acid Sequence |
|---|---|
| 5 | DSWKEEVIKLCGRELVRAQIAICGKST |
| 6 | DSWMEEVIKLCGRELVRAQIAICGKST |
| 7 | DSWQEEVIKLCGRELVRAQIAICGKST |

### Human Relaxin-2 A Chain Derivatives

The disclosure provides human relaxin-2 A chain derivatives, wherein the derivatives have 1, 2, 3, 4, or 5 amino acid changes when compared to the amino acid sequence of SEQ ID NO: 2. In some embodiments, the amino acid that corresponds with position 3 of SEQ ID NO: 2 must be lysine. In some embodiments, the amino acid that corresponds with position 23 of SEQ ID NO: 2 must be phenylalanine. In some embodiments, the amino acid that corresponds with position 3 of SEQ ID NO: 2 must be lysine; and the amino acid that corresponds with position 23 of SEQ ID NO: 2 must be phenylalanine.

In some embodiments, the human relaxin-2 A chain derivatives comprise or consist of the following formula: X₅QX₆YSALANKCCHVGCTKRSLAX₇FC (SEQ ID NO: 4), wherein X₅, X₆, and X₇ are absent or any amino acid. In some embodiments, X₅ is arginine or absent, X₆ is leucine or aspartate, and X₇ is arginine, glutamine, or glutamate.

In some embodiments, the human relaxin-2 A chain derivatives are from 19, 20, 21, 22, 23, 24, 25, 26, 27, 28, 29, or 30 amino acids in length. In some embodiments, the human relaxin-2 A chain derivatives are 22, 23, 24, 25, or 26 amino acids in length. In some embodiments, the human relaxin-2 A chain derivatives are 24 amino acids in length. In some embodiments, the human relaxin-2 A chain derivatives are 25 amino acids in length.

In some embodiments, the human relaxin-2 A chain derivatives comprise or consist of the amino acid sequences shown in Table 2, below.

TABLE 2
Human Relaxin-2 A Chain Derivative Sequences

| SEQ ID NO: | Amino Acid Sequence |
|---|---|
| 8 | RQLYSALANKCCHVGCTKRSLARFC |
| 9 | QLYSALANKCCHVGCTKRSLARFC |
| 10 | RQLYSALANKCCHVGCTKRSLAQFC |
| 11 | RQLYSALANKCCHVGCTKRSLAEFC |
| 12 | RQDYSALANKCCHVGCTKRSLAQFC |
| 13 | RQDYSALANKCCHVGCTKRSLARFC |

### Linker Peptides

The disclosure provides linker peptides, wherein the peptides have at least two acidic amino acids. In some embodiments, the acidic amino acid is glutamate. In some embodiments, the acidic amino acid is aspartate. In some embodiments, the acidic amino acid is a non-standard amino acid. In some embodiments, the acidic amino acid is 2-aminoadipic acid, 2-aminobutyric acid or 2-aminopimelic acid. In some embodiments, the linker peptide has 2, 3, 4, 5, 6, 7, 8, 9, or 10 acidic amino acids.

In some embodiments, the linker peptide is 8, 9, 10, 11, 12, 13, 14, or 15 amino acids in length. In some embodiments, the linker peptide is 12, 13, 14, or 15 amino acids in length. In some embodiments, the linker peptide has 2, 3, 4, or 5 acidic amino acids. In some embodiments, the linker peptide is 12, 13, 14, or 15 amino acids in length and has 2, 3, 4, or 5 acidic amino acids.

In some embodiments, the remaining amino acids are non-acidic amino acids. In some embodiments, the non-acidic amino acids can be any standard amino acid that is not aspartate or glutamate. In some embodiments, non-acidic amino acids can be any amino acid that does not have a carboxylic acid in its side chain. In some embodiments, the non-acidic amino acid is glycine, proline, serine, arginine, histidine, lysine, threonine, asparagine, glutamine, cysteine, alanine, valine, isoleucine, leucine, methionine, phenylalanine, tyrosine, or tryptophan. In some embodiments, the non-acidic amino acid is glycine, proline, or cysteine. In some embodiments, the non-acidic amino acid is glycine.

In some embodiments, the linker peptide comprises acidic amino acids, wherein all the acidic amino acids are the same amino acids. In some embodiments, the acidic amino acids in the linker peptide are both/all glutamates. In some embodiments, the acidic amino acids in the linker peptide are both/all aspartates. In some embodiments, the linker peptide comprises amino acids that are a mixture of acidic amino acids. In some embodiments, the linker peptide comprises both glutamate and aspartate as acidic amino acids.

In some embodiments, the linker peptide comprises an amino acid sequence selected from the group consisting of

R₁R₁R₁R₂R₁R₁R₁R₂R₁R₁R₁R₂R₁;
R₁R₁R₁R₂R₁R₁R₁R₂R₁R₁R₁R₂R₁R₁R₁;
R₁R₁R₂R₁R₁R₁R₂R₂R₁R₁R₁R₂R₁R₁;
R₁R₁R₁R₂R₂R₁R₁R₁R₂R₂R₁R₁R₁; and
R₁R₁R₂R₁R₂R₁R₁R₂R₁R₂R₁R₁R₁,

wherein R₁ is a non-acidic amino acid and R₂ is an acidic amino acid.

In some embodiments, the linker peptide comprises non-acidic amino acids, wherein all the non-acidic amino acids are the same amino acids. In some embodiments, the non-acidic amino acids in the linker peptide are all glycine. In some embodiments, the linker peptide comprises amino acids that are a mixture of non-acidic amino acids. In some embodiments, the linker peptide comprises 2, 3, 4, 5, 6, 7, 8, 9, or 10 different types of non-acidic amino acids.

In some embodiments, the linker peptide comprises or consists of the amino acid sequences shown in Table 3, below.

TABLE 3
Linker Peptide Sequences

| SEQ ID NO: | Amino Acid Sequence |
|---|---|
| 14 | GGGE |
| 15 | GEGE |
| 16 | GGEG |
| 17 | GEGG |
| 18 | GGEE |
| 19 | GGGEGGGEGGGEG |
| 20 | GGGEGGGEGGGEGGG |
| 21 | GEGGGEEGGGEGG |
| 22 | GGGEEGGGEEGGG |
| 23 | GGEGEGGEGEGGS |

In some embodiments, the linker peptide comprises 2, 3, 4, or 5 repeats of SEQ ID NO: 14, 15, 16, 17, or 18. For example, 3 repeats of SEQ ID NO: 14 would be the amino acid sequence of GGGEGGGEGGGE (SEQ ID NO: 24).

### Relaxin/Linker Peptide Combinations for the Fusion Protein

In some embodiments, the fusion protein comprises an N-terminal or first peptide, a linker peptide, and a C-terminal or second peptide. In some embodiments, the N-terminal peptide comprises a human relaxin-2 A chain or a derivative thereof and the C-terminal peptide comprises a human relaxin-2 B chain or a derivative thereof. In some embodiments, the N-terminal peptide comprises a human relaxin-2 B chain or a derivative thereof and the C-terminal peptide comprises a human relaxin-2 A chain or a derivative thereof. Any combination of any of the embodiments of the human relaxin-2 A chain or a derivative thereof, with a human relaxin-2 A chain or a derivative thereof linked by any of the linker peptides disclosed herein can be used to construct embodiments of the fusion proteins described herein. In some embodiments, at least one of the N-terminal peptide and the C-terminal peptide is a derivative of a human relaxin-2 A chain or a human relaxin-2 B chain. In some embodiments, the N-terminal peptide comprises a human relaxin-2 A chain derivative and the C-terminal peptide comprises a human relaxin-2 B chain derivative. In some embodiments, the N-terminal peptide comprises a human relaxin-2 B chain derivative and the C-terminal peptide comprises a human relaxin-2 A chain derivative. Specific embodiments of the fusion proteins provided in this disclosure are shown below in Table 4.

TABLE 4
Fusion Proteins

| B-SEQ ID NO: 19-A | | | B-SEQ ID NO: 20-A | | |
|---|---|---|---|---|---|
| SEQ ID NO | SEQ ID NO | SEQ ID NO | SEQ ID NO | SEQ ID NO | SEQ ID NO |
| of N-terminal | of linker | of C-terminal | of N-terminal | of linker | of C-terminal |
| Peptide | peptide | Peptide | Peptide | peptide | Peptide |
| 5 | 19 | 8 | 5 | 20 | 8 |
| 6 | 19 | 8 | 6 | 20 | 8 |
| 7 | 19 | 8 | 7 | 20 | 8 |
| 5 | 19 | 9 | 5 | 20 | 9 |
| 6 | 19 | 9 | 6 | 20 | 9 |
| 7 | 19 | 9 | 7 | 20 | 9 |
| 5 | 19 | 10 | 5 | 20 | 10 |
| 6 | 19 | 10 | 6 | 20 | 10 |
| 7 | 19 | 10 | 7 | 20 | 10 |
| 5 | 19 | 11 | 5 | 20 | 11 |
| 6 | 19 | 11 | 6 | 20 | 11 |
| 7 | 19 | 11 | 7 | 20 | 11 |
| 5 | 19 | 12 | 5 | 20 | 12 |
| 6 | 19 | 12 | 6 | 20 | 12 |
| 7 | 19 | 12 | 7 | 20 | 12 |
| 5 | 19 | 13 | 5 | 20 | 13 |
| 6 | 19 | 13 | 6 | 20 | 13 |
| 7 | 19 | 13 | 7 | 20 | 13 |
| B-SEQ ID NO: 21-A | | | B-SEQ ID NO: 22-A | | |
| SEQ ID NO | SEQ ID NO | SEQ ID NO | SEQ ID NO | SEQ ID NO | SEQ ID NO |
| of N-terminal | of linker | of C-terminal | of N-terminal | of linker | of C-terminal |
| Peptide | peptide | Peptide | Peptide | peptide | Peptide |
| 5 | 21 | 8 | 5 | 22 | 8 |
| 6 | 21 | 8 | 6 | 22 | 8 |
| 7 | 21 | 8 | 7 | 22 | 8 |
| 5 | 21 | 9 | 5 | 22 | 9 |
| 6 | 21 | 9 | 6 | 22 | 9 |
| 7 | 21 | 9 | 7 | 22 | 9 |
| 5 | 21 | 10 | 5 | 22 | 10 |
| 6 | 21 | 10 | 6 | 22 | 10 |
| 7 | 21 | 10 | 7 | 22 | 10 |
| 5 | 21 | 11 | 5 | 22 | 11 |
| 6 | 21 | 11 | 6 | 22 | 11 |
| 7 | 21 | 11 | 7 | 22 | 11 |
| 5 | 21 | 12 | 5 | 22 | 12 |
| 6 | 21 | 12 | 6 | 22 | 12 |
| 7 | 21 | 12 | 7 | 22 | 12 |
| 5 | 21 | 13 | 5 | 22 | 13 |
| 6 | 21 | 13 | 6 | 22 | 13 |
| 7 | 21 | 13 | 7 | 22 | 13 |
| B-SEQ ID NO: 23-A | | | A-SEQ ID NO: 19-B | | |
| SEQ ID NO | SEQ ID NO | SEQ ID NO | SEQ ID NO | SEQ ID NO | SEQ ID NO |
| of N-terminal | of linker | of C-terminal | of N-terminal | of linker | of C-terminal |
| Peptide | peptide | Peptide | Peptide | peptide | Peptide |
| 5 | 23 | 8 | 8 | 19 | 5 |
| 6 | 23 | 8 | 8 | 19 | 6 |
| 7 | 23 | 8 | 8 | 19 | 7 |
| 5 | 23 | 9 | 9 | 19 | 5 |
| 6 | 23 | 9 | 9 | 19 | 6 |
| 7 | 23 | 9 | 9 | 19 | 7 |
| 5 | 23 | 10 | 10 | 19 | 5 |
| 6 | 23 | 10 | 10 | 19 | 6 |
| 7 | 23 | 10 | 10 | 19 | 7 |
| 5 | 23 | 11 | 11 | 19 | 5 |
| 6 | 23 | 11 | 11 | 19 | 6 |
| 7 | 23 | 11 | 11 | 19 | 7 |
| 5 | 23 | 12 | 12 | 19 | 5 |
| 6 | 23 | 12 | 12 | 19 | 6 |
| 7 | 23 | 12 | 12 | 19 | 7 |
| 5 | 23 | 13 | 13 | 19 | 5 |
| 6 | 23 | 13 | 13 | 19 | 6 |
| 7 | 23 | 13 | 13 | 19 | 7 |
| A-SEQ ID NO: 20-B | | | A-SEQ ID NO: 21-B | | |
| SEQ ID NO | SEQ ID NO | SEQ ID NO | SEQ ID NO | SEQ ID NO | SEQ ID NO |
| of N-terminal | of linker | of C-terminal | of N-terminal | of linker | of C-terminal |
| Peptide | peptide | Peptide | Peptide | peptide | Peptide |
| 8 | 20 | 5 | 8 | 21 | 5 |
| 8 | 20 | 6 | 8 | 21 | 6 |
| 8 | 20 | 7 | 8 | 21 | 7 |
| 9 | 20 | 5 | 9 | 21 | 5 |
| 9 | 20 | 6 | 9 | 21 | 6 |
| 9 | 20 | 7 | 9 | 21 | 7 |
| 10 | 20 | 5 | 10 | 21 | 5 |
| 10 | 20 | 6 | 10 | 21 | 6 |
| 10 | 20 | 7 | 10 | 21 | 7 |
| 11 | 20 | 5 | 11 | 21 | 5 |
| 11 | 20 | 6 | 11 | 21 | 6 |
| 11 | 20 | 7 | 11 | 21 | 7 |
| 12 | 20 | 5 | 12 | 21 | 5 |
| 12 | 20 | 6 | 12 | 21 | 6 |
| 12 | 20 | 7 | 12 | 21 | 7 |
| 13 | 20 | 5 | 13 | 21 | 5 |
| 13 | 20 | 6 | 13 | 21 | 6 |
| 13 | 20 | 7 | 13 | 21 | 7 |
| A-SEQ ID NO: 22-B | | | A-SEQ ID NO: 23-B | | |
| SEQ ID NO | SEQ ID NO | SEQ ID NO | SEQ ID NO | SEQ ID NO | SEQ ID NO |
| of N-terminal | of linker | of C-terminal | of N-terminal | of linker | of C-terminal |
| Peptide | peptide | Peptide | Peptide | peptide | Peptide |
| 8 | 22 | 5 | 8 | 23 | 5 |
| 8 | 22 | 6 | 8 | 23 | 6 |
| 8 | 22 | 7 | 8 | 23 | 7 |
| 9 | 22 | 5 | 9 | 23 | 5 |
| 9 | 22 | 6 | 9 | 23 | 6 |
| 9 | 22 | 7 | 9 | 23 | 7 |
| 10 | 22 | 5 | 10 | 23 | 5 |
| 10 | 22 | 6 | 10 | 23 | 6 |
| 10 | 22 | 7 | 10 | 23 | 7 |
| 11 | 22 | 5 | 11 | 23 | 5 |
| 11 | 22 | 6 | 11 | 23 | 6 |
| 11 | 22 | 7 | 11 | 23 | 7 |
| 12 | 22 | 5 | 12 | 23 | 5 |
| 12 | 22 | 6 | 12 | 23 | 6 |
| 12 | 22 | 7 | 12 | 23 | 7 |
| 13 | 22 | 5 | 13 | 23 | 5 |
| 13 | 22 | 6 | 13 | 23 | 6 |
| 13 | 22 | 7 | 13 | 23 | 7 |

In some embodiments, there are additional amino acids between the N-terminal peptide and the linker peptide. In some embodiments, there are additional amino acids between the C-terminal peptide and the linker peptide. In some embodiments, there are no additional amino acids between the N-terminal peptide and the linker peptide. In some embodiments, there are no additional amino acids between the C-terminal peptide and the linker peptide.

In some embodiments, the portion of the fusion protein comprising the N-terminal peptide, the linker peptide, and the C-terminal peptide comprises or consists of the amino acid sequences shown in Table 5, below.

TABLE 5
Peptide Combinations for the Fusion Protein

| SEQ | |
|---|---|
| ID NO: | Amino Acid Sequence |
| 25 | DSWKEEVIKLCGRELVRAQIAICGKSTGGGEGGGEGGGEGRQLYSALANKCCHVGCTK |
| | RSLARFC |
| 26 | DSWKEEVIKLCGRELVRAQIAICGKSTGGGEGGGEGGGEGGGQLYSALANKCCHVGCT |
| | KRSLARFC |
| 27 | DSWMEEVIKLCGRELVRAQIAICGKSTGGGEGGGEGGGEGRQLYSALANKCCHVGCTK |
| | RSLARFC |
| 28 | DSWQEEVIKLCGRELVRAQIAICGKSTGGGEGGGEGGGEGRQLYSALANKCCHVGCTK |
| | RSLARFC |
| 29 | DSWMEEVIKLCGRELVRAQIAICGKSTGGGEGGGEGGGEGGGQLYSALANKCCHVGCT |
| | KRSLARFC |
| 30 | DSWQEEVIKLCGRELVRAQIAICGKSTGGGEGGGEGGGEGGGQLYSALANKCCHVGCT |
| | KRSLARFC |
| 31 | DSWMEEVIKLCGRELVRAQIAICGKSTGGEGGGEEGGGEGGQLYSALANKCCHVGCTK |
| | RSLARFC |
| 32 | DSWQEEVIKLCGRELVRAQIAICGKSTGGEGGGEEGGGEGGQLYSALANKCCHVGCTK |
| | RSLARFC |
| 33 | DSWMEEVIKLCGRELVRAQIAICGKSTGGEGGGEEGGGEGGQLYSALANKCCHVGCTK |
| | RSLARFC |
| 34 | DSWQEEVIKLCGRELVRAQIAICGKSTGGEGGGEEGGGEGGQLYSALANKCCHVGCTK |
| | RSLARFC |
| 35 | DSWQEEVIKLCGRELVRAQIAICGKSTGGGEGGGEGGGEGRQLYSALANKCCHVGCTK |
| | RSLAQFC |
| 36 | DSWQEEVIKLCGRELVRAQIAICGKSTGGGEGGGEGGGEGRQLYSALANKCCHVGCTK |
| | RSLARFC |
| 37 | DSWQEEVIKLCGRELVRAQIAICGKSTGGGEGGGEGGGEGRQLYSALANKCCHVGCTK |
| | RSLAQFC |
| 38 | DSWQEEVIKLCGRELVRAQIAICGKSTGGGEEGGGEEGGGRQLYSALANKCCHVGCTK |
| | RSLARFC |
| 39 | DSWQEEVIKLCGRELVRAQIAICGKSTGGGEEGGGEEGGGRQLYSALANKCCHVGCTK |
| | RSLAQFC |
| 40 | DSWQEEVIKLCGRELVRAQIAICGKSTGGGEEGGGEEGGGRQLYSALANKCCHVGCTK |
| | RSLARFC |
| 41 | DSWQEEVIKLCGRELVRAQIAICGKSTGGGEEGGGEEGGGRQLYSALANKCCHVGCTK |
| | RSLAQFC |
| 42 | DSWQEEVIKLCGRELVRAQIAICGKSTGGGEGGGEGGGEGRQDYSALANKCCHVGCTK |
| | RSLAQFC |
| 43 | DSWQEEVIKLCGRELVRAQIAICGKSTGGGEGGGEGGGEGRQDYSALANKCCHVGCTK |
| | RSLARFC |
| 44 | DSWQEEVIKLCGRELVRAQIAICGKSTGGGEGGGEGGGEGRQDYSALANKCCHVGCTK |
| | RSLAQFC |
| 45 | DSWQEEVIKLCGRELVRAQIAICGKSTGGEGEGGEGEGGSRQLYSALANKCCHVGCTK |
| | RSLAQFC |
| 46 | DSWQEEVIKLCGRELVRAQIAICGKSTGGEGEGGEGEGGSRQLYSALANKCCHVGCTK |
| | RSLARFC |
| 47 | DSWQEEVIKLCGRELVRAQIAICGKSTGGEGEGGEGEGGSRQLYSALANKCCHVGCTK |
| | RSLAQFC |
| 48 | DSWQEEVIKLCGRELVRAQIAICGKSTGGEGEGGEGEGGSRQDYSALANKCCHVGCTK |
| | RSLAQFC |

### IgG Fc

In some embodiments, the fusion proteins provided herein further comprise an IgG Fc. The IgG Fc can be linked to the N-terminal end of the N-terminal peptide or the C-terminal end of the C-terminal peptide. The IgG Fc can be linked directly to the N-terminal peptide or the C-terminal peptide or they can be linked to the N-terminal peptide or the C-terminal peptide through an IgG Fc linker. In some embodiments, the IgG Fc linker comprises or consists of 1, 2, 3, 4, 5, 6, 7, 8, 9, or 10 amino acids. In some embodiments, the IgG Fc linker comprises or consists of 1, 2, 3, 4, or 5 amino acids. In some embodiments, the IgG Fc linker comprises or consists of 3 or 4 amino acids. In some embodiments, the IgG Fc linker comprises or consists of the amino acid sequence of GGS. It is known in the art that the C-terminal lysine (K) in many monoclonal antibodies is flexible, and is often clipped off during expression and purification with no known impairment in activity. In some embodiments, the IgG Fc comprises the amino acid sequence of one of SEQ ID NOs: 49-52 with GGS as the IgG Fc linker at the C-terminal end of the IgG Fc. In some embodiments, the IgG Fc comprises the amino acid sequence of one of SEQ ID NOs: 200-203 with GGS as the IgG Fc linker at the C-terminal end of the IgG Fc.

In some embodiments, one, two, or more mutations (e.g., amino acid substitutions) are introduced into the Fc region of an antibody described herein (e.g., CH2 domain (residues 231-340 of human IgG1) and/or CH3 domain (residues 341-447 of human IgG1)) and/or the hinge region, numbered according to the EU numbering system, to alter one or more functional properties of the antibody, such as serum half-life, complement fixation, Fc receptor binding, and/or antigen-dependent cellular cytotoxicity.

In certain embodiments, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425, herein incorporated by reference in its entirety. The number of cysteine residues in the hinge region of the CH1 domain may be altered to, e.g., facilitate assembly of the light and heavy chains, or to alter (e.g., increase or decrease) the stability of the antibody.

In a specific embodiment, one, two, or more amino acid mutations (e.g., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375, and 6,165,745, all of which are herein incorporated by reference in their entireties, for examples of mutations that will alter (e.g., decrease or increase) the half-life of an antibody in vivo. In certain embodiments, one, two, or more amino acid mutations (e.g., substitutions, insertions, or deletions) are introduced into an IgG constant domain, or FcRn-binding fragment thereof (preferably an Fc or hinge-Fc domain fragment) to decrease the half-life of the antibody in vivo. In other embodiments, one, two, or more amino acid mutations (e.g., substitutions, insertions, or deletions) are introduced into an IgG constant domain, or FcRn-binding fragment thereof (preferably an Fc or hinge-Fc domain fragment) to increase the half-life of the antibody in vivo. In a specific embodiment, the antibodies may have one or more amino acid mutations (e.g., substitutions) in the second constant (CH2) domain (residues 231-340 of human IgG1) and/or the third constant (CH3) domain (residues 341-447 of human IgG1), numbered according to the EU numbering system. In a specific embodiment, the constant region of the IgG1 of an antibody described herein comprises a methionine (M) to tyrosine (Y) substitution in position 252, a serine (S) to threonine (T) substitution in position 254, and a threonine (T) to glutamic acid (E) substitution in position 256, numbered according to the EU numbering system. See, U.S. Pat. No. 7,658,921, which is herein incorporated by reference in its entirety. This type of mutant IgG, referred to as “YTE mutant” has been shown to display fourfold increased half-life as compared to wild-type versions of the same antibody (see, Dall'Acqua W F et al., (2006) J Biol Chem 281: 23514-24, which is herein incorporated by reference in its entirety). In certain embodiments, an antibody comprises an IgG constant domain comprising one, two, three or more amino acid substitutions of amino acid residues at positions 251-257, 285-290, 308-314, 385-389, and 428-436, numbered according to the EU numbering system.

In certain embodiments, one, two, or more mutations (e.g., amino acid substitutions) are introduced into the Fc region of an antibody described herein (e.g., CH2 domain (residues 231-340 of human IgG1) and/or CH3 domain (residues 341-447 of human IgG1)) and/or the hinge region, numbered according to the EU numbering system, to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, all of which are herein incorporated by reference in their entireties.

In certain embodiments, the antibody comprises a heavy chain constant region that is a variant of a wild-type heavy chain constant region, wherein the variant heavy chain constant region binds to FcγRIIB with higher affinity than the wild-type heavy chain constant region binds to FcγRIIB. In certain embodiments, the variant heavy chain constant region is a variant human heavy chain constant region, e.g., a variant human IgG1, a variant human IgG2, or a variant human IgG4 heavy chain constant region. In certain embodiments, the variant human IgG heavy chain constant region comprises one or more of the following amino acid mutations, according to the EU numbering system: G236D, P238D, S239D, S267E, L328F, and L328E. In certain embodiments, the variant human IgG heavy chain constant region comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F, according to the EU numbering system. In certain embodiments, the FcγRIIB is expressed on a cell selected from the group consisting of macrophages, monocytes, B cells, dendritic cells, endothelial cells, and activated T cells.

In a further embodiment, one, two, or more amino acid substitutions are introduced into an IgG constant domain Fc region to alter the effector function(s) of the antibody. For example, one or more amino acids selected from amino acid residues 234, 235, 236, 237, 239, 243, 267, 292, 297, 300, 318, 320, 322, 328, 330, 332, and 396, numbered according to the EU numbering system, can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, each of which is herein incorporated by reference in its entirety. In certain embodiments, the deletion or inactivation (through point mutations or other means) of a constant region domain may reduce Fc receptor binding of the circulating antibody thereby increasing tumor localization. See, e.g., U.S. Pat. Nos. 5,585,097 and 8,591,886, each of which is herein incorporated by reference in its entirety, for a description of mutations that delete or inactivate the constant domain and thereby increase tumor localization. In certain embodiments, one or more amino acid substitutions may be introduced into the Fc region of an antibody described herein to remove potential glycosylation sites on the Fc region, which may reduce Fc receptor binding (see, e.g., Shields R L et al., (2001) J Biol Chem 276: 6591-604, which is herein incorporated by reference in its entirety). In various embodiments, one or more of the following mutations in the constant region of an antibody described herein may be made: an N297A substitution; an N297Q substitution; an L234A substitution; an L234F substitution; an L235A substitution; an L235F substitution; an L235V substitution; an L237A substitution; an S239D substitution; an E233P substitution; an L234V substitution; an L235A substitution; a C236 deletion; a P238A substitution; an S239D substitution; an F243L substitution; a D265A substitution; an S267E substitution; an L328F substitution; an R292P substitution; a Y300L substitution; an A327Q substitution; a P329A substitution (PA); an A332L substitution; an 1332E substitution; or a P396L substitution, numbered according to the EU numbering system.

In certain embodiments, a mutation selected from the group consisting of D265A, P329A, and a combination thereof, numbered according to the EU numbering system, may be made in the constant region of an antibody described herein. In certain embodiments, a mutation selected from the group consisting of L235A, L237A, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein. In certain embodiments, a mutation selected from the group consisting of S239D, I332E, optionally A330L, and a combination thereof, numbered according to the EU numbering system, may be made in the constant region of an antibody described herein. In certain embodiments, a mutation selected from the group consisting of L235V, F243L, R292P, Y300L, P396L, and a combination thereof, numbered according to the EU numbering system, may be made in the constant region of an antibody described herein. In certain embodiments, a mutation selected from the group consisting of S267E, L328F, and a combination thereof, numbered according to the EU numbering system, may be made in the constant region of an antibody described herein.

In a specific embodiment, an antibody described herein comprises the constant domain of an IgG1 with an N297Q or N297A amino acid substitution, numbered according to the EU numbering system. In one embodiment, an antibody described herein comprises the constant domain of an IgG1 with a mutation selected from the group consisting of D265A, P329A, and a combination thereof, numbered according to the EU numbering system. In another embodiment, an antibody described herein comprises the constant domain of an IgG1 with a mutation selected from the group consisting of L234A, L235A (LALA), and a combination thereof, numbered according to the EU numbering system. In another embodiment, an antibody described herein comprises the constant domain of an IgG1 with a mutation selected from the group consisting of L234F, L235F, N297A, and a combination thereof, numbered according to the EU numbering system. In certain embodiments, amino acid residues in the constant region of an antibody described herein in the positions corresponding to positions L234, L235, and D265 in a human IgG1 heavy chain, numbered according to the EU numbering system, are not L, L, and D, respectively. This approach is described in detail in International Publication No. WO 14/108483, which is herein incorporated by reference in its entirety. In a particular embodiment, the amino acids corresponding to positions L234, L235, and D265 in a human IgG1 heavy chain are F, E, and A; or A, A, and A, respectively, numbered according to the EU numbering system.

In certain embodiments, one or more amino acids selected from amino acid residues 329, 331, and 322 in the constant region of an antibody described herein, numbered according to the EU numbering system, can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al.), which is herein incorporated by reference in its entirety. In certain embodiments, one or more amino acid residues within amino acid positions 231 to 238 in the N-terminal region of the CH2 domain of an antibody described herein are altered to thereby alter the ability of the antibody to fix complement, numbered according to the EU numbering system. This approach is described further in International Publication No. WO 94/29351, which is herein incorporated by reference in its entirety. In certain embodiments, the Fc region of an antibody described herein is modified to increase the ability of the antibody to mediate antibody dependent cellular cytotoxicity (ADCC) and/or to increase the affinity of the antibody for an Fcγ receptor by mutating one or more amino acids (e.g., introducing amino acid substitutions) at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 328, 329, 330, 331, 333, 334, 335, 337, 338, 340, 360, 373, 376, 378, 382, 388, 389, 398, 414, 416, 419, 430, 434, 435, 437, 438, or 439, numbered according to the EU numbering system. This approach is described further in International Publication No. WO 00/42072, which is herein incorporated by reference in its entirety.

In some embodiments, the IgG Fc is an IgG1 Fc, or a derivative thereof. In some embodiments, the IgG Fc or IgG1 Fc comprises an amino acid sequence at least 85, 90, 95, 96, 97, 98, or 99% identical to the amino acid sequence of IgG1 Fc. In some embodiments, the IgG Fc or IgG1 Fc comprises an amino acid sequence at least 85, 90, 95, 96, 97, 98, 99, or 100% identical to an amino acid sequence provided below in Table 6.

TABLE 6
IgG Fc Amino Acid Sequences

| SEQ | | |
|---|---|---|
| ID NO: | Description | Sequence |
| 49 | IgG1 Fc | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 50 | IgG1 Fc | DKTHTCPPCPAPEAAGGPSVFLFPPKPKDTLMISRTPEVTCVVVDV |
| | LALA | SHEDPEVKFNWYVDGVEVHNAKTKPREEQYNSTYRVVSVLTVLHQD |
| | | WLNGKEYKCKVSNKALPAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 51 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | | WLNGKEYKCKVSNKALAAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 52 | IgG1 Fc | DKTHTCPPCPAPEAAGGPSVFLFPPKPKDTLMISRTPEVTCVVVDV |
| | LALA PA LS | SHEDPEVKFNWYVDGVEVHNAKTKPREEQYNSTYRVVSVLTVLHQD |
| | | WLNGKEYKCKVSNKALAAPIEKTISKAKGQPREPQVYTLPPSRDEL |
| | | TKNQVSLTCLVKGFYPSDIAVEWESNGQPENNYKTTPPVLDSDGSF |
| | | FLYSKLTVDKSRWQQGNVFSCSVLHEALHSHYTQKSLSLSPGK |
| 200 | IgG1 Fc | DKTHTCPPCPAPELLGGPSVFLFPPKPKDTLMISRTPEVTCVVVDV |
| | without | SHEDPEVKFNWYVDGVEVHNAKTKPREEQYNSTYRVVSVLTVLHQD |
| | C- | WLNGKEYKCKVSNKALPAPIEKTISKAKGQPREPQVYTLPPSRDEL |
| | terminal | TKNQVSLTCLVKGFYPSDIAVEWESNGQPENNYKTTPPVLDSDGSF |
| | lysine | FLYSKLTVDKSRWQQGNVFSCSVMHEALHNHYTQKSLSLSPG |
| 201 | IgG1 Fc | DKTHTCPPCPAPEAAGGPSVFLFPPKPKDTLMISRTPEVTCVVVDV |
| | LALA | SHEDPEVKFNWYVDGVEVHNAKTKPREEQYNSTYRVVSVLTVLHQD |
| | without | WLNGKEYKCKVSNKALPAPIEKTISKAKGQPREPQVYTLPPSRDEL |
| | C- | TKNQVSLTCLVKGFYPSDIAVEWESNGQPENNYKTTPPVLDSDGSF |
| | terminal | FLYSKLTVDKSRWQQGNVFSCSVMHEALHNHYTQKSLSLSPG |
| | lysine | |
| 202 | IgG1 Fc | DKTHTCPPCPAPEAAGGPSVFLFPPKPKDTLMISRTPEVTCVVVDV |
| | LALA PA | SHEDPEVKFNWYVDGVEVHNAKTKPREEQYNSTYRVVSVLTVLHQD |
| | without | WLNGKEYKCKVSNKALAAPIEKTISKAKGQPREPQVYTLPPSRDEL |
| | C- | TKNQVSLTCLVKGFYPSDIAVEWESNGQPENNYKTTPPVLDSDGSF |
| | terminal | FLYSKLTVDKSRWQQGNVFSCSVMHEALHNHYTQKSLSLSPG |
| | lysine | |
| 203 | IgG1 Fc | DKTHTCPPCPAPEAAGGPSVFLFPPKPKDTLMISRTPEVTCVVVDV |
| | LALA PA LS | SHEDPEVKFNWYVDGVEVHNAKTKPREEQYNSTYRVVSVLTVLHQD |
| | without | WLNGKEYKCKVSNKALAAPIEKTISKAKGQPREPQVYTLPPSRDEL |
| | C- | TKNQVSLTCLVKGFYPSDIAVEWESNGQPENNYKTTPPVLDSDGSF |
| | terminal | FLYSKLTVDKSRWQQGNVFSCSVLHEALHSHYTQKSLSLSPG |
| | lysine | |

In some embodiments, any IgG Fc, or derivative thereof, can be linked to the N-terminus or C-terminus of any of the embodiments described in Table 4 or 5 above with or without an IgG Fc linker. In some embodiments, human IgG1 Fc, or a derivative thereof, can be linked to the N-terminus or C-terminus of any of the embodiments described in Table 4 or 5 above with or without an IgG Fc linker. In some embodiments, the amino acid sequence of the human IgG1 Fc comprises or consists of the amino acid sequence of SEQ ID NO: 49 or 200. In some embodiments, the derivative if human IgG1 Fc comprises an amino acid sequence at least 85, 90, 95, 96, 97, 98, or 99% identical to the amino acid sequence of SEQ ID NO: 49 or 200.

In some embodiments, a human IgG1 Fc comprising a LALA mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA mutation comprises or consists of the amino acid sequence of SEQ ID NO: 50 or 201. In some embodiments, the derivative if human IgG1 Fc comprising a LALA mutation comprises an amino acid sequence at least 85, 90, 95, 96, 97, 98, or 99% identical to the amino acid sequence of SEQ ID NO: 50 or 201.

In some embodiments, a human IgG1 Fc comprising a LALA PA mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA PA mutation comprises or consists of the amino acid sequence of SEQ ID NO: 51 or 202. In some embodiments, the derivative if human IgG1 Fc comprising a LALA PA mutation comprises an amino acid sequence at least 85, 90, 95, 96, 97, 98, or 99% identical to the amino acid sequence of SEQ ID NO: 51 or 202.

In some embodiments, a human IgG1 Fc comprising a LALA PA LS mutation, or a derivative thereof, can be linked to the N-terminus or C-terminus of any of the embodiments described in Table 4 or 5 above with or without an IgG Fc linker. In some embodiments, the amino acid sequence of the human IgG1 Fc comprising a LALA PA LS mutation comprises or consists of the amino acid sequence of SEQ ID NO: 52 or 203. In some embodiments, the derivative if human IgG1 Fc comprising a LALA PA LS mutation comprises an amino acid sequence at least 85, 90, 95, 96, 97, 98, or 99% identical to the amino acid sequence of SEQ ID NO: 52 or 203.

In some embodiments, the fusion protein comprises or consists of the amino acid sequences shown in Table 7, below.

TABLE 7
Fusion Protein Amino Acid Sequences

| SEQ | |
|---|---|
| ID NO: | Sequence |
| 53 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWKEEVIKLCGRELVRA |
| | QIAICGKSTGGGEGGGEGGGEGRQLYSALANKCCHVGCTKRSLARFC |
| 54 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWKEEVIKLCGRELVRA |
| | QIAICGKSTGGGEGGGEGGGEGGGQLYSALANKCCHVGCTKRSLARFC |
| 55 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWMEEVIKLCGRELVRA |
| | QIAICGKSTGGGEGGGEGGGEGRQLYSALANKCCHVGCTKRSLARFC |
| 56 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGGEGGGEGGGEGRQLYSALANKCCHVGCTKRSLARFC |
| 57 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEGGGEGGGEGRQLYSALANKCCHVGCTKRSLARFC |
| 58 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWMEEVIKLCGRELVRA |
| | QIAICGKSTGGGEGGGEGGGEGGGQLYSALANKCCHVGCTKRSLARFC |
| 59 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGGEGGGEGGGEGGGQLYSALANKCCHVGCTKRSLARFC |
| 60 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEGGGEGGGEGGGQLYSALANKCCHVGCTKRSLARFC |
| 61 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWMEEVIKLCGRELVRA |
| | QIAICGKSTGGEGGGEEGGGEGGQLYSALANKCCHVGCTKRSLARFC |
| 62 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGEGGGEEGGGEGGQLYSALANKCCHVGCTKRSLARFC |
| 63 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWMEEVIKLCGRELVRAQ |
| | IAICGKSTGGEGGGEEGGGEGGQLYSALANKCCHVGCTKRSLARFC |
| 64 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWQEEVIKLCGRELVRAQ |
| | IAICGKSTGGEGGGEEGGGEGGQLYSALANKCCHVGCTKRSLARFC |
| 65 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGGEGGGEGGGEGRQLYSALANKCCHVGCTKRSLAQFC |
| 66 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEGGGEGGGEGRQLYSALANKCCHVGCTKRSLAQFC |
| 67 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWQEEVIKLCGRELVRAQ |
| | IAICGKSTGGGEGGGEGGGEGRQLYSALANKCCHVGCTKRSLARFC |
| 68 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEGGGEGGGEGRQLYSALANKCCHVGCTKRSLARFC |
| 69 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWQEEVIKLCGRELVRAQ |
| | IAICGKSTGGGEGGGEGGGEGRQLYSALANKCCHVGCTKRSLAQFC |
| 70 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEGGGEGGGEGRQLYSALANKCCHVGCTKRSLAQFC |
| 71 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGGEEGGGEEGGGRQLYSALANKCCHVGCTKRSLARFC |
| 72 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEEGGGEEGGGRQLYSALANKCCHVGCTKRSLARFC |
| 73 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWQEEVIKLCGRELVRA |
| | QIAICGKSTGGGEEGGGEEGGGRQLYSALANKCCHVGCTKRSLAQFC |
| 74 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEEGGGEEGGGRQLYSALANKCCHVGCTKRSLAQFC |
| 75 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWQEEVIKLCGRELVRAQ |
| | IAICGKSTGGGEEGGGEEGGGRQLYSALANKCCHVGCTKRSLARFC |
| 76 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEEGGGEEGGGRQLYSALANKCCHVGCTKRSLARFC |
| 77 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGSDSWQEEVIKLCGRELVRAQ |
| | IAICGKSTGGGEEGGGEEGGGRQLYSALANKCCHVGCTKRSLAQFC |
| 78 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEEGGGEEGGGRQLYSALANKCCHVGCTKRSLAQFC |
| 79 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGGEGGGEGGGEGRQDYSALANKCCHVGCTKRSLAQFC |
| 80 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEGGGEGGGEGRQDYSALANKCCHVGCTKRSLARFC |
| 81 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGGEGGGEGGGEGRQDYSALANKCCHVGCTKRSLAQFC |
| 82 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGEGEGGEGEGGSRQLYSALANKCCHVGCTKRSLAQFC |
| 83 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGEGEGGEGEGGSRQLYSALANKCCHVGCTKRSLARFC |
| 84 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGGGSDSWQEEVIKLCGRELVRAQ |
| | IAICGKSTGGEGEGGEGEGGSRQLYSALANKCCHVGCTKRSLAQFC |
| 85 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALAAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVLHEALHSHYTQKSLSLSPGKGGSDSWQEEVIKLCGRELVRA |
| | QIAICGKSTGGEGEGGEGEGGSRQDYSALANKCCHVGCTKRSLAQFC |
| 191 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWKEEVIKLCGRELVRA |
| | QIAICGKSTASDAAGANANAGARQLYSALANKCCHVGCTKRSLAQFC |
| 192 | DKTHTCPPCPAPEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGGSDSWKEEVIKLCGRELVRA |
| | QIAICGKSTASDAAGANANAGARQLYSALANKCCHVGCTKRSLAEFC |
| 86 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWKEEVIKLCGRELVRAQIAICGKSTGGGEGGGEGGGEGRQLYSALANKCCHVGCTKRS |
| | LARFC |
| 87 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWKEEVIKLCGRELVRAQIAICGKSTGGGEGGGEGGGEGGGQLYSALANKCCHVGCTKR |
| | SLARFC |
| 88 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWMEEVIKLCGRELVRAQIAICGKSTGGGEGGGEGGGEGRQLYSALANKCCHVGCTKRS |
| | LARFC |
| 89 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGGEGGGEGGGEGRQLYSALANKCCHVGCTKRS |
| | LARFC |
| 90 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEGGGEGGGEGRQLYSALANKCCHVGCTKRS |
| | LARFC |
| 91 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWMEEVIKLCGRELVRAQIAICGKSTGGGEGGGEGGGEGGGQLYSALANKCCHVGCTKR |
| | SLARFC |
| 92 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGGEGGGEGGGEGGGQLYSALANKCCHVGCTKR |
| | SLARFC |
| 93 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEGGGEGGGEGGGQLYSALANKCCHVGCTKR |
| | SLARFC |
| 94 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWMEEVIKLCGRELVRAQIAICGKSTGGEGGGEEGGGEGGQLYSALANKCCHVGCTKRS |
| | LARFC |
| 95 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGEGGGEEGGGEGGQLYSALANKCCHVGCTKRS |
| | LARFC |
| 96 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWMEEVIKLCGRELVRAQIAICGKSTGGEGGGEEGGGEGGQLYSALANKCCHVGCTKRSL |
| | ARFC |
| 97 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWQEEVIKLCGRELVRAQIAICGKSTGGEGGGEEGGGEGGQLYSALANKCCHVGCTKRSL |
| | ARFC |
| 98 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGGEGGGEGGGEGRQLYSALANKCCHVGCTKRS |
| | LAQFC |
| 99 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEGGGEGGGEGRQLYSALANKCCHVGCTKRS |
| | LAQFC |
| 100 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWQEEVIKLCGRELVRAQIAICGKSTGGGEGGGEGGGEGRQLYSALANKCCHVGCTKRSL |
| | ARFC |
| 101 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEGGGEGGGEGRQLYSALANKCCHVGCTKRSL |
| | ARFC |
| 102 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWQEEVIKLCGRELVRAQIAICGKSTGGGEGGGEGGGEGRQLYSALANKCCHVGCTKRSL |
| | AQFC |
| 103 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEGGGEGGGEGRQLYSALANKCCHVGCTKRSL |
| | AQFC |
| 104 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGGEEGGGEEGGGRQLYSALANKCCHVGCTKRS |
| | LARFC |
| 105 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEEGGGEEGGGRQLYSALANKCCHVGCTKRS |
| | LARFC |
| 106 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGKG |
| | GSDSWQEEVIKLCGRELVRAQIAICGKSTGGGEEGGGEEGGGRQLYSALANKCCHVGCTKRS |
| | LAQFC |
| 107 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEEGGGEEGGGRQLYSALANKCCHVGCTKRS |
| | LAQFC |
| 108 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWQEEVIKLCGRELVRAQIAICGKSTGGGEEGGGEEGGGRQLYSALANKCCHVGCTKRSL |
| | ARFC |
| 109 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEEGGGEEGGGRQLYSALANKCCHVGCTKRSL |
| | ARFC |
| 110 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGGG |
| | SDSWQEEVIKLCGRELVRAQIAICGKSTGGGEEGGGEEGGGRQLYSALANKCCHVGCTKRSL |
| | AQFC |
| 111 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEEGGGEEGGGRQLYSALANKCCHVGCTKRSL |
| | AQFC |
| 112 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGGEGGGEGGGEGRQDYSALANKCCHVGCTKRS |
| | LAQFC |
| 113 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEGGGEGGGEGRQDYSALANKCCHVGCTKRSL |
| | ARFC |
| 114 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGGEGGGEGGGEGRQDYSALANKCCHVGCTKRSL |
| | AQFC |
| 115 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGEGEGGEGEGGSRQLYSALANKCCHVGCTKRS |
| | LAQFC |
| 116 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGEGEGGEGEGGSRQLYSALANKCCHVGCTKRSL |
| | ARFC |
| 117 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGGG |
| | SDSWQEEVIKLCGRELVRAQIAICGKSTGGEGEGGEGEGGSRQLYSALANKCCHVGCTKRSL |
| | AQFC |
| 118 | METDTLLLWVLLLWVPGSTGDKTHTCPPCPAPEAAGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVS |
| | NKALAAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVLHEALHSHYTQKSLSLSPGKG |
| | GSDSWQEEVIKLCGRELVRAQIAICGKSTGGEGEGGEGEGGSRQDYSALANKCCHVGCTKRS |
| | LAQFC |

As shown in Table 7, above, in some embodiments, the IgG Fc comprises a mouse IgG kappa signal sequence comprising the amino acid sequence of METDTLLLWVLLLWVPGSTG (SEQ ID NO: 194). In some embodiments a different signal sequence is used. In some embodiments, some shown in Table 7, no signal sequence is present on the fusion protein as produced.

### Other Half-Life Extending Moieties

As used herein, the term “half-life extending moiety” includes non-proteinaceous, half-life extending moieties, such as PEG or HES, and proteinaceous half-life extending moieties such as Fc domain. In some embodiments, non-proteinaceous half-life extending moieties are linked to the fusion proteins described herein. In some embodiments, the non-proteinaceous half-life extending moieties are linked to the fusion proteins instead of IgG Fc. In some embodiments, the non-proteinaceous half-life extending moieties are linked to the fusion proteins in addition to IgG Fc.

Examples of suitable polymer molecules that act as non-proteinaceous half-life extending moieties include polymer molecules selected from the group consisting of polyalkylene oxide (PAO), including polyalkylene glycol (PAG), such as polyethylene glycol (PEG) and polypropylene glycol (PPG), branched PEGs, hydroxyalkyl starch (HAS), such as hydroxyethyl starch (HES), polysialic acid (PSA), poly-vinyl alcohol (PVA), poly-carboxylate, poly-(vinylpyrrolidone), polyethylene-co-maleic acid anhydride, polystyrene-co-maleic acid anhydride, dextran, including carboxymethyl-dextran, or any other biopolymer suitable for reducing immunogenicity and/or increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms.

PEG has the advantage of having only few reactive groups capable of cross-linking compared to, e.g., polysaccharides such as dextran. In particular, monofunctional PEG, e.g., methoxypolyethylene glycol (mPEG), is of interest since its coupling chemistry is relatively simple (only one reactive group is available for conjugating with attachment groups on the polypeptide). Consequently, as the risk of cross-linking is eliminated, the resulting conjugated fusion proteins described herein are more homogeneous, and the reaction of the polymer molecules with the variant polypeptide is easier to control.

To effect covalent attachment of the polymer molecule(s) to the fusion proteins described herein, the hydroxyl end groups of the polymer molecule must be provided in activated form, i.e., with reactive functional groups (examples of which include primary amino groups, hydrazide (HZ), thiol, succinate (SUC), succinimidyl succinate (SS), succinimidyl succinamide (SSA), succinimidyl propionate (SPA), succinimidyl butyrate (SBA), succinimidyl carboxymethylate (SCM), benzotriazole carbonate (BTC), N-hydroxysuccinimide (NHS), aldehyde, nitrophenylcarbonate (NPC), and tresylate (TRES)). Suitable activated polymer molecules are commercially available, e.g., from Shearwater Polymers, Inc., Huntsville, Ala., USA, or from PolyMASC Pharmaceuticals plc, UK.

Alternatively, the polymer molecules can be activated by conventional methods known in the art, e.g., as disclosed in WO 90/13540. Specific examples of activated linear or branched polymer molecules for use herein are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g., SPA-PEG, SSPA-PEG, SBA-PEG, SS-PEG, SSA-PEG, SC-PEG, SG-PEG, and SCM-PEG), and NOR-PEG, BTC-PEG, EPOXPEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. Nos. 5,932,462 and 5,643,575, both of which are incorporated herein by reference.

Furthermore, the following publications disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO 95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503, and EP 154 316.

Specific examples of activated PEG polymers particularly preferred for coupling to cysteine residues, include the following linear PEGs: vinylsulfone-PEG (VS-PEG), preferably vinylsulfone-mPEG (VS-mPEG); maleimide-PEG (MAL-PEG), preferably maleimide-mPEG (MAL-mPEG) and orthopyridyl-disulfide-PEG (OPSS-PEG), preferably orthopyridyl-disulfide-mPEG (OPSS-mPEG). Typically, such PEG or mPEG polymers will have a size of about 5 kDa, about 10 kDa, about 12 kDa or about 20 kDa.

The conjugation of the fusion proteins described herein and the activated polymer molecules is conducted by use of any conventional method, e.g., as described in the following references (which also describe suitable methods for activation of polymer molecules): Harris and Zalipsky, eds., Poly(ethylene glycol) Chemistry and Biological Applications, AZC Washington; R. F. Taylor, (1991), “Protein immobilisation. Fundamental and applications,” Marcel Dekker, N.Y.; S. S. Wong, (1992), “Chemistry of Protein Conjugation and Crosslinking,” CRC Press, Boca Raton; G. T. Hermanson et al., (1993), “Immobilized Affinity Ligand Techniques”, Academic Press, N.Y.

The skilled person will be aware that the activation method and/or conjugation chemistry to be used depends on the attachment group(s) of the fusion protein (examples of which are given further above), as well as the functional groups of the polymer (e.g., being amine, hydroxyl, carboxyl, aldehyde, sulfhydryl, succinimidyl, maleimide, vinylsulfone or haloacetate). The PEGylation may be directed towards conjugation to all available attachment groups on the fusion protein (i.e., such attachment groups that are exposed at the surface of the polypeptide) or may be directed towards one or more specific attachment groups, e.g., the N-terminal amino group as described in U.S. Pat. No. 5,985,265 or to cysteine residues. Furthermore, the conjugation may be achieved in one step or in a stepwise manner (e.g., as described in WO 99/55377).

For PEGylation to cysteine residues (see above) the fusion protein is usually treated with a reducing agent, such as dithiothreitol (DDT) prior to PEGylation. The reducing agent is subsequently removed by any conventional method, such as by desalting. Conjugation of PEG to a cysteine residue typically takes place in a suitable buffer at pH 6-9 at temperatures varying from 4° C. to 25° C. for periods up to 16 hours.

It will be understood that the PEGylation is designed so as to produce the optimal molecule with respect to the number of PEG molecules attached, the size and form of such molecules (e.g., whether they are linear or branched), and the attachment site(s) in the fusion protein. The molecular weight of the polymer to be used may e.g., be chosen on the basis of the desired effect to be achieved.

In connection with conjugation to only a single attachment group on the fusion protein (e.g., the N-terminal amino group), it may be advantageous that the polymer molecule, which may be linear or branched, has a high molecular weight, preferably about 10-25 kDa, such as about 15-25 kDa, e.g., about 20 kDa.

Normally, the polymer conjugation is performed under conditions aimed at reacting as many of the available polymer attachment groups with polymer molecules. This is achieved by means of a suitable molar excess of the polymer relative to the polypeptide. Typically, the molar ratios of activated polymer molecules to polypeptide are up to about 1000-1, such as up to about 200-1, or up to about 100-1. In some cases the ratio may be somewhat lower, however, such as up to about 50-1, 10-1, 5-1, 2-1 or 1-1 in order to obtain optimal reaction.

It is also contemplated to couple the polymer molecules to the fusion protein through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are blocked according to methods known in the art, e.g., by addition of primary amine to the reaction mixture, and the resulting inactivated polymer molecules are removed by a suitable method.

It will be understood that depending on the circumstances, e.g., the amino acid sequence of the fusion protein, the nature of the activated PEG compound being used and the specific PEGylation conditions, including the molar ratio of PEG to polypeptide, varying degrees of PEGylation may be obtained, with a higher degree of PEGylation generally being obtained with a higher ratio of PEG to fusion protein. The PEGylated fusion proteins resulting from any given PEGylation process will, however, normally comprise a stochastic distribution of conjugated fusion protein having slightly different degrees of PEGylation.

For improvement of the biological half-life of the fusion proteins described herein, chemical modification such as PEGylation, or HESylation are applicable.

HAS and HES non-proteinaceous polymers, as well as methods of producing HAS or HES conjugates are disclosed for example in WO 02/080979, WO 03/070772, WO 057092391 and WO 057092390.

Polysialytion is another technology, which uses the natural polymer polysialic acid (PSA) to prolong the half-life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the fusion protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

### Biological Activity of the Relaxin-2 Fusion Proteins

In some embodiments, the relaxin-2 fusion proteins described herein have high levels of biological activity as compared to native relaxin-2. In some embodiments, any of the relaxin-2 fusion proteins described herein have from about 1% to about 200% of a biological activity as compared to native relaxin-2. In some embodiments, the relaxin-2 fusion protein has at least about 5%, about 10%, about 15%, about 20%, about 25%, about 30%, about 35%, about 40%, about 50%, about 60%, about 70%, about 80%, about 90%, about 100%, about 125% about 150%, about 175%, or about 200% of a biological activity as compared to native relaxin-2.

In some embodiments, any of the relaxin-2 fusion proteins described herein have from about 1% to about 200% of maximal biological activity as compared to native relaxin-2. In some embodiments, maximal biological activity is the maximum response (Eₘₐₓ) of relaxin-2 or relaxin-2 fusion protein. In some embodiments, the relaxin-2 fusion protein has at least about 5%, about 10%, about 15%, about 20%, about 25%, about 30%, about 35%, about 40%, about 50%, about 60%, about 70%, about 80%, about 90%, about 100%, about 125% about 150%, about 175%, or about 200% of a maximal biological activity as compared to native relaxin-2.

In some embodiments, any of the relaxin-2 fusion proteins described herein have about at least about 0.001-fold to about at least 1,000-fold enhanced potency as compared to native relaxin-2. In some embodiments, potency is the concentration of relaxin-2 or relaxin-2 fusion protein to elicit a half-maximal response (EC₅₀). In some embodiments, the relaxin-2 fusion protein has at least about 0.001-fold, about 0.01-fold, about 0.1-fold, about 1-fold, about 10-fold, about 100-fold, or about 1,000-fold of the potency as compared to native relaxin-2.

The biological activity can be any biological activity of native relaxin-2. For example, the biological activity can be the capacity to bind the receptor of native relaxin-2, RXFP1. The binding of relaxin-2 to RXFP1 can be measured by any well-known methods in the art, such as radioligand binding. In some embodiments, the fusion proteins described herein bind to RXFP1 when it is expressed on a cell surface.

In some embodiments, the biological activity can be the capacity to activate RXFP1 on a cell surface. The activation of RXFP1 by the relaxin-2 fusion proteins described herein can be determined by the increase of cAMP using any methods well known in the art, such as measuring the activity of a cAMP-driven reporter gene, e.g., 0-galactosidase. The activation of RXFP1 by the relaxin-2 fusion proteins described herein in a cell may also be determined by using a biosensor such as the GloSensor biosensor. The activation of RXFP1 by the relaxin-2 fusion proteins described herein in a cell may also be determined by measuring the expression of certain genes, such as angiogenic factors, e.g., VEGF, or the expression of MMPs using well-known methods in the art. In some embodiments, the biological activity is a physiological, biochemical activity or any other effect-inducing activity of the relaxin-2. Exemplary biological activities include, but are not limited to, vasodilation, collagen degradation, angiogenesis, decreasing arterial blood pressure, increasing renal artery blood flow, increasing cardiac filling at diastole, resolving established fibrosis, and suppressing new fibrosis development.

In some embodiments, the fusion proteins described herein have improved pharmacokinetics profiles. Without wishing to be bound by any theory, the structure of the fusion proteins described herein is based upon, at least in part, the surprising discovery that reducing the pI of relaxin-2 fusion protein analogs increases their circulating half-life. In some embodiments, the circulating half-life is in a mammal. In some embodiments, the mammal is a rodent or a primate. In some embodiments, the rodent is a rat or a mouse. In some embodiments, the primate is a human or a monkey. In some embodiments, the monkey is a cynomolgus monkey. In some embodiments, the mammal is a human. In some embodiments, the fusion proteins described herein may have a circulating half-life of greater than about 5 hours, 10 hours, 20 hours, 50 hours, 75 hours, 100 hours, 125 hours, 150 hours, or more. In some embodiments, the fusion proteins described herein may have a circulating half-life of 5-10 hours, 10-20 hours, 20-50 hours, 50-75 hours, 75-100 hours, 100-125 hours, or 125-150 hours. Values and ranges intermediate to the recited values are also intended to be part of this disclosure. In some embodiments, the fusion proteins described herein have a longer circulating half-life than a native two chain relaxin-2. For example, the circulating half-life of a native two chain relaxin-2 may be less than about 5 hours. (See, e.g., Chen et al., The Pharmacokinetics of Recombinant Human Relaxin in Non-Pregnant Women after Intravenous, Intravaginal, and Intracervical Administration, Pharm. Res. 10: 834038 (1993), incorporated herein by reference).

This is increased half-life can be, at least in part, attributed to the reduced pI of the fusion proteins described herein. In some embodiments, the pI of the fusion protein is less than 9.4. In some embodiments, the pI of the fusion protein is less than 9.0, 8.9, 8.8, 8.7, 8.6, 8.5, 8.4, 8.3, 8.2, 8.1, or 8.0. In some embodiments, the pI of the fusion proteins described herein are between 6.0 and 9.4. In some embodiments, the pI of the fusion proteins described herein are 6.5-8.5, 6.6-8.4, 6.7-8.3, 6.8-8.2, 6.8-8.1, 6.8-8.0, or 6.8-7.9. In some embodiments, the pI referred to above is the calculated or theoretical pI. In some embodiments, the pI referred to above is the experimentally measured pI.

“Circulating half-life,” as used herein, refers to the time it takes for the blood plasma concentration of a drug to halve its steady-state when circulating in the full blood of an organism. Circulating half-life of a particular agent may vary depending on a multitude of factors including, but not limited to, dosage, formulation, and/or administration route of the agent. One of ordinary skill in the art is able to determine the circulating half-life of an agent using well known methods in the art, such as the method described Chen supra.

### Vectors

The disclosure also provides nucleic acid molecules that encode any of the fusion proteins or peptides described herein. In some embodiments, the nucleic acid molecules described herein are DNA molecules. In some embodiments, the nucleic acid molecules described herein are RNA molecules.

The nucleic acid molecules described herein can be transcribed from a promoter in an expression vector. In some embodiments, the vector is a non-viral vector. Exemplary non-viral vectors include, but are not limited to, plasmid DNA, transposons, episomal plasmids, minicircles, ministrings, and oligonucleotides (e.g., mRNA, naked DNA). In some embodiments, the vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picomnavirus vectors (e.g., Coxsackievirus).

In some embodiments, the vector or expression cassette comprises one or more additional elements. Additional elements include, but are not limited to, promoters, enhancers, polyadenylation (polyA) sequences, and selection genes.

In some embodiments, the vector comprises a polynucleotide sequence that encodes an amino acid sequence at least 90%, 95%, 96%, 97%, 98%, 99%, or 100% identical to an amino acid sequence recited in any of Tables 1-7. In some embodiments, the vector comprises or consists of a nucleotide sequence at least 75%, 80%, 85%, 90%, 95%, 96%, 97%, 98%, 99%, or 100% identical to any of the nucleotide sequence recited in Table 8, below.

TABLE 8
Nucleotide Sequences Encoding Fusion Proteins and Peptide Components

| SEQ | |
|---|---|
| ID NO: | Sequence |
| 119 | GATAAAACACACACGTGTCCCCCCTGCCCGGCTCCAGAGGCGGCTGGTGGTCCCAGCGTATT |
| | CTTGTTTCCTCCCAAACCTAAGGATACGCTCATGATATCCCGCACCCCAGAAGTTACGTGTG |
| | TAGTCGTCGACGTCAGTCACGAAGATCCAGAGGTCAAATTTAACTGGTATGTCGACGGAGTA |
| | GAGGTCCACAATGCGAAAACCAAGCCCAGAGAAGAGCAGTACAACTCCACGTATCGCGTCGT |
| | CTCCGTCCTCACCGTACTCCATCAAGATTGGCTGAATGGGAAAGAGTATAAATGCAAAGTAT |
| | CTAACAAGGCTCTGCCAGCTCCGATAGAAAAGACTATATCAAAGGCCAAGGGGCAGCCAAGG |
| | GAGCCTCAAGTCTATACTTTGCCCCCATCTCGGGATGAGCTTACGAAAAACCAGGTCAGCCT |
| | TACCTGTCTTGTTAAAGGTTTTTATCCGAGTGACATCGCAGTGGAATGGGAATCTAATGGTC |
| | AACCTGAAAACAATTACAAAACCACACCGCCAGTATTGGACAGCGATGGTAGTTTTTTTCTT |
| | TACTCAAAACTGACTGTAGATAAAAGCAGATGGCAGCAGGGCAATGTCTTTTCATGTAGCGT |
| | TATGCATGAGGCTCTTCACAACCACTATACCCAAAAGTCATTGTCTCTTAGTCCCGGAAAGG |
| | GCGGAAGTGATTCTTGGAAGGAGGAGGTAATCAAGTTGTGCGGGCGAGAGTTGGTACGGGCA |
| | CAGATCGCGATATGCGGAAAATCCACAGGTGGGGGCGAAGGAGGAGGTGAGGGTGGAGGTGA |
| | AGGACGACAGTTGTATTCCGCCTTGGCAAACAAGTGTTGCCATGTGGGTTGCACAAAACGCA |
| | GTCTTGCCCGCTTCTGT |
| 120 | GATAAGACACATACATGCCCTCCCTGTCCGGCTCCAGAGGCAGCCGGGGGTCCATCAGTCTT |
| | CCTTTTTCCGCCTAAACCTAAGGATACACTGATGATCTCTCGAACACCGGAGGTCACTTGTG |
| | TTGTCGTTGACGTATCACATGAGGATCCCGAAGTAAAGTTCAACTGGTATGTCGATGGTGTG |
| | GAGGTTCATAATGCTAAAACTAAACCACGGGAGGAGCAATATAATTCCACATATAGGGTCGT |
| | GAGCGTGTTGACGGTGCTTCATCAAGACTGGCTTAATGGGAAGGAATATAAATGCAAAGTGT |
| | CAAATAAAGCACTTCCTGCGCCAATCGAGAAAACAATTAGTAAGGCAAAGGGGCAGCCGCGA |
| | GAACCTCAGGTGTACACCTTGCCGCCTTCTAGAGACGAGCTCACAAAGAACCAAGTTTCCCT |
| | GACTTGCCTCGTTAAGGGGTTTTATCCGTCCGATATAGCCGTGGAGTGGGAGTCAAACGGCC |
| | AACCGGAAAATAATTACAAAACGACACCCCCAGTATTGGATAGTGACGGCTCTTTTTTCCTT |
| | TATTCTAAGCTGACTGTGGACAAAAGCCGCTGGCAGCAGGGCAATGTCTTTTCATGCAGCGT |
| | AATGCATGAAGCCCTGCACAACCACTACACGCAAAAATCCCTTTCCTTGTCACCCGGCAAGG |
| | GCGGCTCTGACTCCTGGAAAGAGGAAGTTATAAAACTCTGTGGCCGAGAACTTGTTCGAGCT |
| | CAAATCGCGATTTGTGGTAAGTCAACGGGTGGGGGCGAAGGTGGAGGCGAGGGTGGGGGAGA |
| | AGGAGGAGGCCAGTTGTACTCAGCTCTTGCAAATAAGTGTTGCCACGTTGGTTGTACGAAGC |
| | GGAGCCTTGCTCGCTTCTGC |
| 121 | GACAAAACACATACTTGTCCGCCTTGCCCGGCACCCGAAGCGGCCGGCGGACCCAGTGTCTT |
| | TCTCTTCCCACCCAAACCGAAAGACACTCTGATGATTTCCAGGACGCCTGAAGTGACCTGCG |
| | TTGTAGTTGATGTATCACACGAGGATCCCGAGGTCAAGTTCAATTGGTATGTAGATGGGGTG |
| | GAGGTCCATAATGCAAAGACGAAGCCACGGGAGGAACAGTACAACTCTACGTACAGAGTTGT |
| | CAGTGTTTTGACCGTCCTTCATCAGGATTGGCTGAACGGTAAAGAATATAAATGCAAGGTTA |
| | GCAATAAAGCTTTGCCCGCCCCTATAGAGAAAACGATCAGTAAGGCGAAGGGGCAGCCTAGG |
| | GAACCCCAGGTATATACCTTGCCGCCAAGTCGAGATGAGCTGACGAAGAACCAAGTGAGTCT |
| | GACATGCCTCGTGAAGGGCTTCTATCCGAGCGATATCGCTGTCGAATGGGAGAGCAATGGGC |
| | AGCCTGAGAATAACTATAAAACAACGCCACCCGTCCTCGACTCCGATGGCTCATTCTTCCTG |
| | TACAGTAAACTTACAGTAGATAAGAGTAGATGGCAGCAGGGTAACGTCTTTAGTTGCTCCGT |
| | GATGCACGAGGCATTGCACAATCATTACACTCAAAAATCTCTGTCCCTGAGTCCGGGCAAAG |
| | GCGGTTCAGATAGCTGGATGGAGGAGGTCATAAAGCTTTGTGGACGAGAACTCGTTCGCGCC |
| | CAGATAGCTATTTGTGGGAAATCAACCGGGGGTGGAGAAGGTGGCGGAGAAGGGGGAGGCGA |
| | AGGGCGCCAACTGTATTCTGCATTGGCTAATAAGTGCTGTCACGTAGGATGTACAAAAAGGT |
| | CTCTGGCGAGATTCTGC |
| 122 | GACAAGACGCACACTTGTCCACCTTGCCCTGCGCCGGAAGCTGCTGGAGGCCCCAGTGTCTT |
| | TTTGTTCCCGCCCAAACCGAAGGACACTTTGATGATAAGTCGCACGCCCGAGGTTACCTGTG |
| | TGGTTGTCGATGTCTCACACGAAGATCCGGAGGTGAAGTTTAATTGGTATGTAGATGGCGTG |
| | GAGGTTCATAACGCCAAAACGAAACCCAGAGAAGAACAATATAACAGTACATATCGAGTAGT |
| | ATCCGTTCTCACTGTCCTGCATCAAGACTGGTTGAACGGGAAGGAATATAAGTGCAAGGTGA |
| | GCAATAAAGCACTCCCGGCCCCAATCGAAAAGACCATCAGCAAAGCGAAGGGGCAACCTCGA |
| | GAACCCCAGGTATATACGCTCCCCCCTAGTCGGGATGAACTTACTAAAAATCAGGTTAGCCT |
| | CACTTGCCTTGTTAAAGGGTTCTATCCCAGTGATATTGCCGTCGAATGGGAATCAAACGGGC |
| | AGCCGGAAAATAACTACAAGACAACCCCTCCTGTGCTCGATAGCGATGGCTCTTTTTTCCTC |
| | TACAGCAAACTTACCGTTGATAAGAGCCGGTGGCAACAAGGTAATGTTTTCTCCTGCTCCGT |
| | TATGCATGAAGCACTCCATAACCATTATACCCAAAAAAGCCTGTCACTTAGTCCGGGTAAAG |
| | GAGGTAGTGATTCTTGGCAGGAGGAGGTAATCAAACTTTGTGGGAGGGAGCTGGTACGAGCT |
| | CAGATTGCTATATGTGGAAAAAGCACGGGCGGAGGAGAAGGAGGTGGCGAAGGCGGGGGTGA |
| | AGGTCGGCAACTCTACTCCGCTCTCGCTAATAAGTGCTGCCACGTCGGGTGTACGAAGCGCT |
| | CCCTGGCGCGATTCTGC |
| 123 | GATAAAACGCACACGTGTCCGCCCTGCCCAGCGCCTGAAGCCGCAGGCGGGCCGTCCGTCTT |
| | CCTCTTTCCTCCAAAACCCAAAGACACACTTATGATCAGTAGGACCCCAGAGGTAACCTGCG |
| | TCGTGGTCGACGTTTCCCATGAAGACCCAGAGGTCAAGTTCAACTGGTACGTCGACGGTGTC |
| | GAAGTACATAATGCTAAAACGAAGCCTCGGGAAGAGCAGTACAACTCTACCTACCGCGTCGT |
| | TTCCGTACTCACCGTACTTCACCAGGACTGGCTTAACGGTAAAGAGTATAAATGCAAAGTAT |
| | CTAATAAGGCTCTCGCCGCGCCGATTGAGAAGACAATTTCAAAGGCCAAGGGGCAGCCGCGG |
| | GAGCCCCAAGTGTATACCTTGCCCCCGTCCCGAGATGAGCTGACTAAAAACCAAGTAAGCTT |
| | GACTTGCTTGGTCAAAGGCTTCTACCCTTCCGATATAGCTGTCGAATGGGAGTCAAATGGCC |
| | AACCAGAGAACAATTATAAAACTACACCCCCGGTCTTGGATTCTGATGGCTCATTTTTTCTC |
| | TATTCTAAACTGACCGTGGATAAGTCTCGCTGGCAGCAAGGTAACGTGTTCAGTTGCTCTGT |
| | TCTTCACGAAGCACTGCACAGTCATTACACTCAGAAGAGTCTTAGCCTGAGCCCTGGTAAAG |
| | GGGGTTCTGATTCCTGGCAGGAGGAAGTAATAAAACTCTGTGGCCGGGAGTTGGTACGGGCG |
| | CAGATTGCGATATGCGGTAAGAGCACCGGCGGAGGCGAAGGCGGTGGGGAAGGAGGAGGAGA |
| | AGGGAGACAACTCTATTCCGCATTGGCAAATAAGTGCTGCCACGTCGGGTGTACCAAACGAT |
| | CCCTTGCACGGTTCTGT |
| 124 | GATAAGACCCATACGTGCCCCCCTTGCCCTGCGCCTGAGGCAGCGGGTGGCCCATCAGTCTT |
| | TTTGTTCCCGCCCAAGCCAAAGGACACCCTCATGATTAGTAGAACACCGGAGGTTACGTGCG |
| | TCGTAGTGGATGTCAGCCACGAGGATCCCGAGGTTAAGTTTAACTGGTACGTTGATGGGGTT |
| | GAGGTCCATAATGCGAAGACTAAGCCGAGAGAGGAACAGTACAATTCCACGTATAGAGTTGT |
| | CTCTGTACTGACTGTGCTGCATCAAGATTGGCTTAACGGTAAGGAGTACAAGTGCAAAGTCT |
| | CTAATAAGGCTCTTCCTGCACCCATTGAGAAAACTATAAGCAAAGCAAAAGGTCAACCTCGC |
| | GAACCTCAGGTGTACACACTGCCACCCTCTAGGGACGAGCTTACCAAAAATCAAGTATCTCT |
| | TACCTGCCTTGTGAAAGGGTTTTATCCCTCAGATATTGCGGTTGAGTGGGAGTCTAACGGAC |
| | AACCTGAGAACAACTATAAGACTACTCCCCCGGTGCTTGATTCAGACGGGAGTTTTTTTTTG |
| | TATAGCAAACTTACCGTCGACAAAAGCCGGTGGCAACAGGGCAATGTATTCAGTTGTTCTGT |
| | AATGCATGAAGCTTTGCATAATCATTACACCCAAAAGAGTCTTTCCCTGTCTCCTGGAAAAG |
| | GGGGGTCAGACTCCTGGATGGAGGAGGTGATCAAACTGTGTGGGAGAGAGCTCGTCCGGGCT |
| | CAGATAGCTATATGCGGCAAGTCTACGGGTGGGGGAGAGGGCGGAGGAGAGGGCGGTGGAGA |
| | AGGAGGCGGCCAACTCTACAGCGCTCTGGCCAATAAATGTTGTCATGTCGGGTGTACTAAGC |
| | GCTCACTGGCACGCTTTTGC |
| 125 | GACAAGACGCATACATGCCCGCCATGCCCGGCCCCCGAAGCTGCTGGGGGACCATCCGTATT |
| | CCTCTTCCCTCCCAAACCAAAAGACACGTTGATGATAAGTAGAACACCAGAGGTAACGTGCG |
| | TGGTTGTCGATGTTTCCCACGAAGATCCGGAGGTAAAATTCAATTGGTATGTAGATGGGGTG |
| | GAAGTGCACAATGCCAAAACAAAGCCGCGAGAAGAACAATACAATAGTACTTACCGGGTTGT |
| | GAGCGTGCTCACGGTGTTGCACCAAGACTGGCTCAACGGCAAGGAATACAAGTGCAAAGTAT |
| | CTAATAAAGCTCTGCCTGCGCCGATAGAGAAGACCATCAGTAAGGCCAAAGGGCAGCCCCGA |
| | GAGCCGCAAGTTTACACTCTTCCTCCGAGCAGAGATGAATTGACCAAGAACCAAGTAAGTTT |
| | GACGTGCCTGGTGAAGGGCTTCTACCCCTCAGACATTGCGGTGGAGTGGGAAAGTAATGGTC |
| | AACCGGAAAACAACTACAAGACCACGCCGCCCGTCCTCGACTCCGATGGGTCTTTCTTTCTT |
| | TATTCAAAGTTGACAGTAGATAAGTCAAGGTGGCAGCAAGGTAACGTGTTTAGTTGTAGTGT |
| | AATGCACGAGGCCCTGCATAATCATTATACCCAAAAGAGTTTGAGCCTCTCACCAGGAAAAG |
| | GCGGATCAGACAGCTGGCAGGAGGAGGTAATTAAATTGTGTGGACGGGAGTTGGTCAGGGCG |
| | CAAATAGCCATCTGCGGTAAGAGCACGGGTGGAGGAGAGGGTGGAGGGGAAGGTGGGGGAGA |
| | AGGCGGCGGGCAGCTCTATTCTGCACTCGCCAACAAGTGTTGTCACGTCGGATGCACAAAGA |
| | GATCTCTTGCTCGATTCTGC |
| 126 | GACAAAACACACACCTGTCCGCCTTGCCCGGCTCCTGAAGCCGCGGGTGGCCCTAGTGTGTT |
| | TTTGTTTCCGCCGAAACCTAAGGATACCCTCATGATAAGCCGGACGCCCGAGGTTACCTGTG |
| | TCGTGGTCGATGTTAGTCATGAGGATCCAGAAGTCAAGTTTAATTGGTACGTCGACGGCGTT |
| | GAAGTCCACAATGCAAAAACTAAACCGCGAGAAGAACAGTACAACTCCACCTACAGAGTTGT |
| | CTCAGTTTTGACAGTTCTCCATCAGGATTGGCTCAATGGAAAGGAATATAAGTGCAAGGTCA |
| | GCAATAAAGCGCTTGCCGCCCCTATAGAGAAGACCATTAGCAAGGCGAAAGGACAGCCCCGC |
| | GAGCCCCAGGTCTATACGCTGCCTCCTAGCAGAGATGAGCTCACGAAAAATCAGGTCAGCTT |
| | GACATGCTTGGTGAAGGGCTTCTACCCCAGTGACATCGCAGTTGAATGGGAGAGCAACGGCC |
| | AACCTGAGAACAACTACAAAACAACGCCCCCGGTTCTTGACAGCGATGGGTCCTTCTTTCTT |
| | TACTCTAAGCTTACAGTTGATAAAAGCAGGTGGCAGCAGGGGAATGTGTTCTCATGTTCCGT |
| | ACTGCATGAGGCTCTGCATTCTCACTACACCCAAAAAAGCCTTAGCCTGAGCCCCGGTAAGG |
| | GAGGTAGTGACTCATGGCAAGAGGAAGTGATTAAGCTCTGCGGCCGGGAGTTGGTGAGAGCC |
| | CAAATCGCCATTTGCGGTAAAAGTACCGGAGGGGGCGAGGGAGGAGGCGAAGGTGGAGGTGA |
| | AGGAGGTGGACAGTTGTACTCAGCTCTTGCAAATAAATGTTGTCATGTTGGTTGCACGAAAA |
| | GATCTCTTGCGAGGTTCTGT |
| 127 | GATAAGACGCATACTTGTCCACCGTGCCCCGCACCGGAAGCGGCTGGTGGTCCATCAGTTTT |
| | TCTGTTCCCACCGAAACCTAAGGACACGTTGATGATATCACGGACACCAGAGGTTACGTGCG |
| | TAGTGGTGGATGTGAGCCACGAGGATCCAGAAGTTAAATTTAATTGGTACGTAGATGGAGTG |
| | GAGGTTCATAATGCGAAGACAAAGCCTCGCGAGGAACAGTATAATTCCACCTATCGCGTCGT |
| | ATCTGTGCTTACGGTACTTCACCAAGACTGGTTGAACGGTAAGGAATATAAATGCAAGGTTT |
| | CCAATAAAGCACTTCCTGCGCCAATTGAGAAGACAATATCCAAAGCTAAAGGTCAACCCAGG |
| | GAACCGCAAGTCTACACTCTCCCCCCGTCTCGCGATGAATTGACGAAGAACCAGGTTAGTCT |
| | CACCTGCCTGGTCAAGGGGTTTTACCCCTCTGACATAGCTGTAGAATGGGAGTCTAATGGAC |
| | AGCCAGAGAACAATTACAAAACGACCCCCCCGGTCCTCGATTCTGATGGGAGTTTTTTTCTT |
| | TATTCAAAATTGACTGTCGATAAGTCAAGATGGCAACAGGGTAACGTATTTTCTTGCAGTGT |
| | TATGCATGAAGCATTGCACAACCACTATACACAAAAATCATTGAGTTTGAGTCCCGGTAAAG |
| | GGGGAAGCGACTCATGGATGGAAGAAGTAATCAAGCTGTGCGGGCGAGAGCTTGTGCGAGCT |
| | CAGATAGCAATCTGTGGTAAGTCTACAGGTGGAGAGGGTGGCGGTGAAGAAGGCGGGGGAGA |
| | GGGAGGCCAGCTTTATTCTGCCCTGGCTAACAAGTGCTGTCACGTTGGATGCACGAAGCGCT |
| | CCCTGGCCCGATTCTGC |
| 128 | GATAAGACGCATACTTGTCCCCCATGTCCCGCTCCGGAAGCCGCTGGCGGCCCCTCCGTTTT |
| | TCTGTTCCCGCCGAAACCGAAAGACACCCTGATGATATCACGCACTCCCGAGGTCACTTGCG |
| | TGGTAGTCGATGTTAGTCATGAAGATCCTGAGGTCAAATTCAATTGGTATGTAGATGGCGTT |
| | GAGGTACACAACGCGAAGACAAAACCCCGAGAAGAACAGTATAACTCAACCTACCGCGTAGT |
| | TTCAGTTCTTACCGTACTGCACCAAGACTGGTTGAACGGTAAAGAGTACAAATGTAAAGTCA |
| | GCAATAAAGCTTTGCCAGCACCTATCGAAAAAACCATCAGTAAGGCCAAGGGTCAACCCAGG |
| | GAGCCGCAAGTGTACACTCTTCCCCCTAGCAGGGATGAATTGACCAAGAATCAGGTCTCTTT |
| | GACGTGCCTCGTTAAGGGTTTCTATCCCAGCGATATAGCCGTAGAATGGGAGTCTAACGGTC |
| | AGCCAGAAAATAACTATAAGACAACCCCGCCTGTTTTGGATTCCGACGGCTCTTTTTTTCTC |
| | TACTCTAAGTTGACCGTTGATAAGAGCAGATGGCAGCAGGGAAACGTATTTTCTTGTTCCGT |
| | GATGCACGAAGCCCTGCACAATCACTATACGCAAAAGTCTCTGAGCTTGAGTCCGGGTAAAG |
| | GCGGTTCTGACTCCTGGCAGGAGGAAGTCATAAAACTCTGCGGAAGAGAGCTCGTAAGGGCG |
| | CAAATCGCTATTTGTGGTAAGAGCACCGGTGGGGAAGGAGGCGGTGAAGAGGGTGGCGGCGA |
| | GGGTGGGCAATTGTATTCCGCGCTTGCCAATAAATGTTGTCACGTAGGCTGCACAAAGCGAA |
| | GTCTCGCTAGGTTCTGC |
| 129 | GACAAGACCCACACATGTCCCCCGTGTCCGGCACCAGAAGCAGCGGGGGGACCGTCAGTATT |
| | CTTGTTTCCACCGAAGCCCAAAGACACATTGATGATTTCACGAACTCCTGAAGTTACCTGTG |
| | TGGTTGTAGATGTATCACACGAAGACCCAGAAGTCAAATTCAATTGGTATGTCGACGGGGTT |
| | GAAGTTCACAATGCGAAGACGAAGCCCCGGGAGGAACAGTACAACAGCACGTACAGGGTTGT |
| | GAGCGTTCTTACTGTATTGCACCAGGATTGGCTCAACGGCAAGGAGTATAAATGTAAAGTTT |
| | CTAATAAGGCTCTTCCTGCCCCAATTGAAAAGACGATATCTAAAGCGAAGGGCCAACCACGG |
| | GAACCTCAGGTGTACACACTTCCGCCTAGCAGGGATGAGTTGACCAAGAATCAAGTCTCTTT |
| | GACGTGCCTGGTCAAGGGGTTTTACCCATCAGATATCGCCGTCGAATGGGAGTCAAACGGAC |
| | AACCCGAAAATAACTATAAAACTACTCCACCAGTTCTGGATAGCGACGGCTCATTTTTTCTG |
| | TATTCAAAGCTCACTGTAGACAAGTCTAGGTGGCAGCAGGGTAATGTCTTCTCCTGCTCAGT |
| | AATGCATGAGGCTCTTCACAACCACTATACTCAAAAGAGCCTTTCCCTGTCACCTGGCGGTG |
| | GAAGCGACTCATGGATGGAGGAGGTAATAAAGCTCTGCGGAAGAGAACTGGTACGCGCACAA |
| | ATCGCAATTTGTGGTAAGAGTACTGGCGGGGAAGGAGGTGGGGAAGAAGGGGGCGGTGAGGG |
| | CGGACAGCTCTATTCTGCACTTGCAAACAAATGTTGCCACGTGGGATGTACTAAGCGAAGCC |
| | TTGCAAGATTCTGC |
| 130 | GATAAAACCCACACATGCCCTCCATGCCCTGCTCCAGAGGCCGCCGGTGGGCCATCAGTTTT |
| | CTTGTTTCCGCCTAAACCAAAGGACACGCTTATGATCTCCAGGACCCCCGAAGTTACGTGTG |
| | TGGTGGTTGATGTTAGTCACGAGGACCCGGAAGTCAAGTTCAACTGGTACGTTGATGGTGTA |
| | GAGGTGCACAATGCAAAGACGAAGCCACGCGAAGAACAATACAACAGCACATATCGAGTTGT |
| | GAGCGTACTCACGGTACTGCATCAGGACTGGCTGAACGGTAAAGAATACAAATGTAAAGTCT |
| | CCAATAAGGCACTTCCTGCGCCGATAGAAAAAACGATCAGTAAGGCCAAGGGCCAACCCCGA |
| | GAACCACAGGTATATACGCTCCCACCGTCACGAGACGAGTTGACAAAAAATCAGGTCTCCCT |
| | GACTTGCCTCGTGAAAGGTTTTTATCCCTCAGATATTGCTGTTGAGTGGGAAAGCAATGGGC |
| | AGCCAGAGAATAATTATAAGACGACTCCTCCGGTTTTGGATTCCGACGGTAGTTTTTTCTTG |
| | TATAGTAAGCTTACTGTAGACAAGTCAAGATGGCAACAAGGTAATGTGTTCTCTTGCTCAGT |
| | TATGCATGAAGCTCTTCATAACCATTACACGCAAAAGAGTCTCAGTCTGAGCCCCGGTGGCG |
| | GTAGCGACAGTTGGCAGGAAGAGGTGATTAAGTTGTGCGGTCGCGAGCTCGTTCGGGCCCAA |
| | ATTGCAATCTGCGGAAAATCTACGGGCGGAGAGGGCGGGGGTGAGGAGGGTGGGGGTGAAGG |
| | TGGGCAGCTCTATAGCGCCCTTGCGAATAAATGTTGTCACGTCGGATGCACAAAGAGGTCCC |
| | TCGCCAGGTTCTGC |
| 131 | GATAAGACCCACACTTGCCCCCCTTGCCCTGCCCCCGAAGCGGCCGGAGGTCCTTCAGTATT |
| | TTTGTTTCCACCGAAACCCAAAGATACTTTGATGATATCAAGAACTCCTGAAGTCACCTGCG |
| | TGGTAGTTGACGTATCTCATGAGGATCCCGAGGTGAAGTTCAATTGGTACGTCGATGGCGTC |
| | GAGGTTCATAACGCTAAGACTAAGCCGAGGGAAGAGCAATATAATTCCACTTATAGGGTGGT |
| | GTCCGTCTTGACTGTTTTGCACCAGGATTGGTTGAACGGGAAAGAGTACAAATGTAAGGTGA |
| | GTAATAAAGCTTTGGCTGCTCCCATCGAAAAGACAATAAGCAAGGCCAAGGGGCAACCTCGG |
| | GAGCCGCAGGTGTACACCCTTCCTCCCAGTAGAGACGAACTGACAAAAAACCAGGTGTCCCT |
| | GACCTGCCTTGTGAAGGGGTTTTACCCGAGCGACATAGCGGTTGAATGGGAGAGCAACGGGC |
| | AACCCGAGAACAACTACAAAACTACACCGCCTGTCCTGGACTCCGATGGAAGCTTCTTCCTC |
| | TACTCCAAACTGACCGTGGACAAAAGCAGATGGCAACAAGGAAACGTATTCTCATGCTCAGT |
| | AATGCACGAAGCATTGCACAATCACTACACCCAAAAGTCCCTCTCACTCTCCCCTGGTAAGG |
| | GCGGATCAGACTCATGGCAAGAGGAGGTAATTAAGTTGTGCGGGAGGGAGCTCGTCCGCGCG |
| | CAAATAGCCATTTGTGGCAAGTCCACTGGAGGAGGCGAGGGTGGAGGAGAGGGTGGTGGGGA |
| | GGGCAGGCAACTCTACAGTGCGCTCGCCAATAAATGCTGCCATGTTGGGTGCACGAAGCGCA |
| | GTCTCGCACAATTCTGC |
| 132 | GATAAGACCCACACGTGTCCTCCATGTCCGGCACCGGAGGCTGCTGGCGGGCCTTCTGTATT |
| | CCTCTTCCCACCCAAGCCAAAAGACACATTGATGATATCAAGGACGCCGGAAGTCACCTGTG |
| | TTGTTGTGGACGTTTCCCATGAAGACCCAGAGGTAAAATTCAATTGGTATGTGGACGGCGTA |
| | GAGGTTCACAACGCCAAAACCAAACCCCGAGAGGAACAGTATAATAGCACATATCGAGTAGT |
| | ATCTGTTCTCACAGTGCTCCATCAAGACTGGCTTAATGGTAAAGAGTATAAATGCAAAGTTT |
| | CCAATAAAGCCCTCGCTGCACCGATCGAGAAGACAATCAGTAAAGCGAAGGGCCAGCCTCGG |
| | GAACCGCAGGTGTATACTCTTCCACCCTCAAGAGACGAGCTCACTAAAAACCAAGTTTCATT |
| | GACATGCCTCGTCAAAGGTTTCTACCCATCAGACATCGCGGTCGAATGGGAAAGTAATGGGC |
| | AGCCGGAAAACAACTATAAAACGACGCCGCCCGTCTTGGATTCTGATGGTTCATTTTTTCTT |
| | TACTCTAAATTGACCGTCGATAAAAGTAGGTGGCAACAAGGAAATGTTTTTTCCTGCTCCGT |
| | CCTGCATGAAGCGTTGCACAGTCACTATACCCAGAAGAGTCTTTCTTTGTCACCCGGAAAAG |
| | GCGGTTCAGATTCATGGCAGGAAGAAGTAATTAAACTCTGTGGCCGCGAGCTTGTTAGGGCG |
| | CAGATAGCCATATGTGGTAAAAGCACCGGAGGAGGTGAAGGCGGAGGCGAAGGAGGTGGGGA |
| | AGGAAGACAATTGTATTCTGCACTTGCAAATAAATGCTGTCATGTGGGGTGCACGAAACGCA |
| | GTCTTGCACAATTTTGT |
| 133 | GACAAAACCCATACCTGCCCCCCTTGCCCTGCACCAGAAGCGGCGGGAGGACCTAGCGTTTT |
| | TCTTTTTCCTCCGAAACCGAAAGATACCCTCATGATATCAAGAACACCTGAGGTTACTTGCG |
| | TTGTCGTGGACGTGAGTCACGAAGACCCCGAGGTGAAGTTCAACTGGTATGTAGATGGAGTG |
| | GAGGTCCATAATGCAAAAACGAAACCGAGAGAAGAACAATACAACTCTACATATCGAGTCGT |
| | GTCAGTACTCACGGTTTTGCATCAAGATTGGCTGAACGGTAAGGAGTACAAGTGTAAGGTTA |
| | GCAACAAGGCTCTCGCGGCGCCGATAGAAAAGACTATAAGTAAAGCAAAAGGCCAGCCCAGA |
| | GAACCTCAAGTTTACACTCTGCCTCCCAGCAGAGATGAACTGACTAAAAATCAGGTTTCATT |
| | GACCTGTCTCGTCAAGGGTTTTTATCCAAGCGACATAGCAGTTGAATGGGAAAGCAACGGTC |
| | AACCAGAAAATAATTACAAAACCACTCCACCAGTCTTGGACTCTGACGGATCCTTCTTTCTC |
| | TATTCAAAATTGACGGTGGATAAATCTAGGTGGCAGCAAGGCAACGTCTTCTCTTGTAGCGT |
| | TATGCATGAGGCGCTGCACAACCACTACACACAAAAGTCTCTTAGTTTGAGCCCGGGCGGCG |
| | GAAGCGACTCTTGGCAAGAGGAAGTGATAAAACTCTGTGGTCGAGAATTGGTACGCGCGCAG |
| | ATCGCTATCTGCGGCAAGTCCACAGGGGGAGGGGAAGGTGGCGGGGAAGGTGGTGGCGAGGG |
| | CAGGCAGTTGTATAGTGCACTTGCCAACAAGTGCTGCCATGTGGGGTGCACCAAGCGCAGTT |
| | TGGCACGGTTCTGC |
| 134 | GATAAAACTCACACTTGTCCCCCGTGTCCGGCACCAGAAGCCGCAGGAGGGCCATCTGTCTT |
| | TCTTTTTCCCCCAAAACCCAAGGATACACTGATGATCTCCCGCACTCCCGAAGTTACTTGTG |
| | TCGTAGTAGACGTTTCTCACGAGGACCCAGAGGTGAAATTCAATTGGTATGTTGACGGAGTA |
| | GAGGTGCATAATGCCAAGACAAAGCCCCGAGAGGAACAATACAATTCAACCTACAGAGTAGT |
| | GTCCGTTCTTACGGTTCTCCATCAGGATTGGCTCAACGGTAAGGAATATAAGTGCAAGGTAA |
| | GCAACAAAGCGCTGGCCGCACCCATTGAGAAAACCATTTCAAAAGCTAAAGGCCAACCCCGC |
| | GAACCACAAGTTTATACTCTCCCCCCAAGTCGCGATGAACTTACAAAAAATCAAGTCTCATT |
| | GACGTGCTTGGTCAAAGGCTTCTACCCGAGCGATATCGCTGTTGAATGGGAGTCTAATGGAC |
| | AACCGGAAAATAACTATAAAACTACACCCCCAGTCCTCGATTCAGACGGCAGCTTCTTCCTG |
| | TATTCAAAACTGACGGTTGACAAATCACGCTGGCAACAGGGTAACGTTTTTTCCTGTAGCGT |
| | TCTTCATGAAGCCTTGCACAGTCACTACACCCAGAAGTCCCTTAGCTTGTCACCTGGCGGGG |
| | GTTCAGACTCTTGGCAGGAGGAGGTAATCAAACTGTGCGGAAGAGAACTGGTGAGGGCTCAG |
| | ATTGCAATTTGTGGGAAGAGCACGGGTGGCGGTGAAGGAGGTGGCGAGGGCGGAGGAGAGGG |
| | GAGGCAACTCTACAGTGCGTTGGCTAATAAATGCTGTCACGTCGGCTGTACTAAGAGAAGCC |
| | TCGCCAGATTTTGC |
| 135 | GACAAGACGCATACTTGCCCTCCGTGCCCTGCACCAGAAGCCGCTGGTGGCCCATCTGTGTT |
| | TTTGTTCCCCCCTAAGCCAAAAGACACATTGATGATTTCACGAACTCCAGAAGTGACTTGCG |
| | TAGTTGTTGACGTATCACACGAAGACCCCGAGGTTAAATTTAATTGGTATGTGGACGGGGTC |
| | GAGGTGCATAACGCCAAAACCAAACCCCGGGAGGAACAATATAACTCTACGTATCGGGTCGT |
| | ATCTGTGTTGACCGTCCTTCACCAAGATTGGTTGAACGGCAAGGAATATAAGTGTAAAGTGT |
| | CTAATAAAGCATTGGCTGCCCCGATAGAAAAGACGATCTCTAAAGCCAAGGGCCAACCCAGA |
| | GAGCCTCAAGTATATACTCTCCCACCGAGTCGAGATGAGCTCACTAAGAACCAGGTGTCACT |
| | CACGTGTCTGGTTAAAGGATTTTACCCTAGTGATATAGCCGTCGAGTGGGAATCAAATGGGC |
| | AGCCGGAGAATAACTATAAGACCACGCCTCCAGTTCTCGATTCCGATGGTAGCTTTTTCCTT |
| | TACTCTAAACTTACGGTCGACAAGTCCAGGTGGCAACAGGGCAATGTATTTTCTTGCTCCGT |
| | CATGCACGAGGCTTTGCACAACCATTACACGCAAAAGTCACTGTCCCTGTCTCCTGGAGGCG |
| | GTTCTGACAGTTGGCAGGAGGAGGTAATCAAATTGTGTGGGCGGGAGTTGGTTAGGGCGCAA |
| | ATTGCTATTTGCGGCAAAAGTACTGGGGGCGGTGAAGGCGGAGGCGAGGGAGGAGGAGAAGG |
| | TCGACAACTGTATTCTGCCTTGGCGAACAAATGCTGTCACGTCGGCTGTACGAAACGGTCTT |
| | TGGCCCAGTTTTGT |
| 136 | GATAAGACACACACTTGTCCGCCATGCCCTGCGCCGGAAGCGGCGGGAGGACCGTCCGTTTT |
| | CCTGTTCCCTCCCAAACCCAAAGACACGTTGATGATTAGTCGCACGCCAGAAGTTACGTGCG |
| | TTGTCGTAGATGTATCCCACGAAGACCCCGAGGTGAAGTTCAATTGGTATGTAGATGGGGTG |
| | GAGGTCCATAACGCTAAGACCAAACCACGCGAGGAACAATATAATTCTACGTACCGCGTAGT |
| | GAGCGTTCTCACAGTTCTTCACCAGGATTGGCTTAACGGCAAGGAGTATAAGTGTAAGGTGT |
| | CTAATAAGGCCTTGGCTGCCCCGATCGAAAAAACGATAAGTAAAGCAAAGGGTCAACCTAGA |
| | GAACCCCAAGTGTACACTCTCCCGCCATCACGGGATGAATTGACTAAGAACCAAGTGTCACT |
| | CACGTGTCTTGTAAAGGGCTTCTACCCATCCGATATAGCCGTTGAGTGGGAATCCAATGGTC |
| | AGCCAGAGAACAACTATAAGACAACTCCGCCCGTACTTGATAGTGACGGTTCCTTTTTCCTT |
| | TACAGTAAATTGACGGTAGATAAGTCTCGCTGGCAGCAAGGAAACGTCTTTTCTTGTTCAGT |
| | GCTTCATGAGGCGCTTCACTCACACTATACTCAGAAGAGTTTGAGTTTGTCTCCAGGTGGAG |
| | GCAGCGACTCATGGCAAGAGGAAGTAATCAAACTGTGTGGTCGCGAATTGGTACGAGCACAG |
| | ATCGCGATCTGCGGGAAATCAACAGGTGGCGGCGAAGGCGGCGGGGAAGGCGGCGGCGAAGG |
| | TAGGCAACTTTACTCAGCCCTTGCGAACAAATGTTGCCACGTAGGCTGTACTAAGAGAAGTC |
| | TCGCCCAGTTTTGC |
| 137 | GACAAGACTCATACCTGCCCCCCTTGTCCAGCACCAGAAGCAGCTGGCGGGCCAAGCGTGTT |
| | CCTGTTTCCACCTAAGCCCAAAGATACGTTGATGATCAGCCGCACCCCGGAAGTAACCTGTG |
| | TAGTAGTAGATGTGTCCCACGAAGACCCCGAAGTAAAGTTTAATTGGTACGTCGATGGTGTC |
| | GAAGTACATAACGCTAAAACGAAGCCCCGAGAAGAGCAGTACAACAGTACTTACAGAGTAGT |
| | TTCTGTTCTTACAGTGCTGCATCAGGATTGGCTGAACGGGAAGGAGTATAAATGTAAAGTCT |
| | CAAACAAGGCACTTGCGGCACCAATAGAGAAGACAATATCTAAGGCCAAAGGGCAGCCTAGA |
| | GAGCCACAAGTATATACGCTGCCCCCCAGCAGGGACGAGCTGACAAAGAACCAAGTGTCACT |
| | GACCTGCCTTGTTAAGGGCTTCTATCCGAGTGATATTGCTGTTGAATGGGAAAGTAACGGAC |
| | AGCCGGAGAACAACTATAAAACTACTCCACCCGTGTTGGATAGTGACGGTAGCTTTTTTCTG |
| | TACTCCAAGTTGACGGTAGACAAAAGTCGGTGGCAGCAGGGGAACGTATTTTCTTGTTCTGT |
| | CATGCACGAAGCTCTTCACAATCACTATACGCAGAAGTCCCTCTCTCTCTCTCCTGGGAAGG |
| | GTGGTTCCGACAGCTGGCAGGAGGAGGTCATTAAACTGTGTGGTAGAGAGCTGGTACGGGCT |
| | CAAATTGCAATTTGTGGTAAGAGTACTGGCGGTGGCGAGGAAGGGGGTGGGGAGGAGGGCGG |
| | AGGTAGGCAGCTCTACTCTGCTCTCGCCAACAAGTGTTGTCACGTCGGGTGTACTAAAAGAT |
| | CACTTGCCCGCTTTTGT |
| 138 | GACAAAACACATACATGCCCGCCGTGTCCGGCGCCTGAAGCAGCAGGAGGCCCCAGTGTATT |
| | CCTTTTCCCTCCAAAGCCAAAAGATACGTTGATGATATCTAGGACACCTGAGGTTACCTGCG |
| | TCGTAGTGGACGTATCCCACGAAGACCCAGAAGTCAAGTTTAACTGGTATGTGGACGGAGTG |
| | GAGGTACACAATGCAAAGACAAAGCCGCGAGAGGAACAATATAATTCCACCTATAGAGTCGT |
| | GTCAGTCCTTACGGTCTTGCACCAGGACTGGCTCAATGGTAAGGAGTATAAGTGCAAAGTAT |
| | CAAACAAAGCTCTCGCAGCGCCCATCGAAAAGACCATCAGCAAAGCTAAGGGCCAGCCAAGA |
| | GAGCCTCAAGTGTACACGTTGCCGCCTTCAAGGGACGAGCTCACTAAAAATCAGGTATCACT |
| | TACGTGTCTTGTCAAAGGGTTTTATCCTTCCGACATCGCGGTTGAATGGGAGAGCAATGGAC |
| | AGCCGGAGAATAATTATAAAACGACGCCGCCGGTCCTTGACAGCGATGGTTCATTTTTCCTT |
| | TACTCAAAGCTGACGGTTGATAAGTCTAGGTGGCAGCAGGGGAACGTCTTTTCCTGTAGTGT |
| | ACTTCATGAGGCGCTCCATTCTCATTACACTCAGAAGTCACTGAGCCTTTCACCCGGCAAAG |
| | GTGGATCAGACTCCTGGCAAGAAGAGGTAATCAAACTCTGTGGGAGGGAACTCGTTCGAGCC |
| | CAGATTGCAATCTGTGGGAAAAGCACAGGCGGAGGGGAAGAAGGGGGTGGCGAAGAAGGTGG |
| | GGGCAGGCAGCTCTATTCAGCTCTTGCCAACAAATGCTGTCATGTAGGCTGCACAAAGCGAT |
| | CACTGGCGAGATTCTGT |
| 139 | GATAAAACTCATACTTGCCCACCCTGCCCGGCTCCCGAGGCAGCAGGTGGACCCTCAGTATT |
| | TTTGTTCCCTCCGAAACCTAAAGATACACTTATGATTAGCCGGACCCCTGAGGTAACGTGTG |
| | TGGTGGTTGACGTAAGTCATGAAGATCCAGAAGTAAAGTTTAACTGGTACGTAGACGGTGTG |
| | GAGGTACATAATGCGAAGACAAAACCACGAGAGGAACAGTATAACTCTACCTACCGCGTAGT |
| | AAGCGTACTTACTGTGCTCCACCAAGACTGGCTTAACGGGAAAGAGTATAAGTGTAAAGTCA |
| | GTAATAAAGCACTGGCCGCCCCGATCGAAAAAACAATCAGCAAGGCCAAAGGACAACCAAGG |
| | GAGCCTCAGGTCTATACTCTTCCCCCGAGTAGGGATGAGCTTACCAAGAACCAGGTGTCTCT |
| | GACATGCCTTGTCAAGGGATTTTACCCGAGTGACATAGCCGTAGAATGGGAGTCAAACGGCC |
| | AACCTGAAAACAACTATAAGACCACGCCTCCCGTACTCGACTCAGATGGAAGCTTTTTCCTC |
| | TATAGCAAGCTGACCGTCGACAAAAGTAGGTGGCAACAGGGAAACGTCTTTAGTTGTTCCGT |
| | CATGCACGAAGCTTTGCATAACCATTACACCCAGAAGAGTCTTTCCCTTTCCCCTGGCAAGG |
| | GGGGCTCCGACTCCTGGCAAGAGGAAGTAATCAAACTGTGTGGGCGCGAGCTTGTCCGCGCG |
| | CAAATAGCCATTTGCGGAAAAAGTACTGGAGGAGGAGAGGAAGGCGGCGGCGAGGAAGGTGG |
| | GGGCAGGCAGCTGTACAGTGCCTTGGCTAACAAGTGCTGCCATGTCGGCTGTACGAAAAGGT |
| | CTCTTGCTCAATTCTGT |
| 140 | GATAAGACACATACCTGTCCACCCTGCCCAGCACCTGAAGCTGCAGGCGGCCCCAGCGTATT |
| | CCTGTTTCCTCCGAAGCCGAAAGACACACTTATGATTTCCCGGACGCCTGAGGTAACTTGCG |
| | TCGTAGTAGATGTGTCTCACGAAGACCCCGAGGTGAAATTCAACTGGTACGTTGATGGTGTG |
| | GAAGTTCATAATGCGAAAACTAAACCACGAGAGGAGCAATATAACTCAACTTATAGAGTTGT |
| | GAGCGTCTTGACGGTACTGCACCAGGACTGGCTGAATGGCAAAGAGTACAAATGCAAAGTCT |
| | CAAATAAGGCGTTGGCGGCTCCCATAGAGAAAACTATCAGCAAAGCCAAGGGTCAACCTCGG |
| | GAGCCACAAGTGTATACTCTTCCGCCTAGTCGCGACGAGCTCACAAAGAATCAGGTGAGTCT |
| | TACTTGTTTGGTTAAGGGTTTCTACCCCAGTGACATTGCGGTCGAGTGGGAAAGTAACGGAC |
| | AGCCTGAAAACAACTATAAAACAACGCCTCCAGTACTCGATTCAGATGGTTCATTCTTTCTT |
| | TATTCCAAACTCACAGTCGACAAGAGTAGATGGCAACAAGGGAACGTGTTTAGCTGTAGCGT |
| | ACTCCATGAGGCACTCCACTCTCACTATACCCAAAAGTCTCTCAGCTTGTCACCCGGAAAAG |
| | GCGGTTCTGACAGTTGGCAAGAGGAAGTGATTAAATTGTGTGGGCGGGAACTTGTGAGGGCT |
| | CAAATCGCGATTTGCGGCAAGTCCACTGGTGGCGGCGAGGAAGGAGGAGGTGAAGAAGGAGG |
| | AGGTAGGCAACTGTATTCAGCGTTGGCGAATAAATGCTGCCATGTTGGATGTACTAAACGGA |
| | GCCTTGCTCAGTTCTGC |
| 141 | GATAAAACGCATACTTGCCCTCCTTGCCCGGCACCTGAAGCTGCCGGAGGTCCTTCCGTGTT |
| | CCTGTTCCCACCTAAGCCAAAAGACACACTTATGATTTCTCGCACACCAGAAGTAACGTGCG |
| | TCGTAGTTGACGTCTCCCATGAAGACCCGGAGGTAAAATTTAATTGGTACGTCGACGGGGTA |
| | GAAGTTCATAACGCAAAGACTAAACCACGAGAAGAGCAATACAACTCTACATACAGAGTAGT |
| | AAGCGTTCTCACCGTTCTTCATCAAGATTGGCTCAACGGAAAGGAGTATAAGTGTAAGGTGT |
| | CCAATAAAGCGTTGGCCGCACCAATCGAAAAGACCATAAGCAAAGCCAAAGGCCAACCCCGC |
| | GAACCGCAGGTGTACACACTTCCCCCGTCCAGGGATGAATTGACAAAAAACCAAGTTTCCCT |
| | CACGTGTCTCGTCAAGGGATTCTACCCGAGTGATATCGCAGTTGAATGGGAAAGCAATGGTC |
| | AGCCCGAGAATAACTACAAGACTACTCCCCCTGTGTTGGACTCAGACGGCTCATTCTTCCTC |
| | TACAGTAAGTTGACTGTGGACAAAAGTCGGTGGCAGCAAGGCAATGTCTTCAGTTGTAGTGT |
| | AATGCATGAAGCACTCCACAATCATTACACCCAAAAATCCCTGAGCCTGTCCCCGGGCGGAG |
| | GTTCAGATTCATGGCAGGAGGAAGTTATAAAACTGTGCGGGCGCGAGTTGGTGAGGGCGCAG |
| | ATCGCAATCTGTGGAAAGAGTACGGGAGGTGGCGAAGAGGGTGGTGGAGAAGAGGGAGGAGG |
| | TCGACAACTGTATTCCGCGCTCGCGAACAAGTGTTGCCACGTTGGCTGCACCAAACGAAGCC |
| | TGGCTCGATTTTGC |
| 142 | GACAAGACACACACTTGTCCACCTTGCCCGGCTCCCGAGGCGGCAGGAGGACCAAGCGTTTT |
| | TCTGTTCCCTCCCAAACCAAAGGATACGCTTATGATCTCTCGAACGCCGGAAGTTACTTGCG |
| | TAGTAGTTGATGTCTCCCATGAAGATCCCGAAGTGAAGTTCAACTGGTATGTAGATGGTGTG |
| | GAAGTTCATAACGCGAAAACCAAACCACGCGAAGAACAGTATAACAGTACTTATCGGGTTGT |
| | TTCAGTACTCACGGTGCTCCATCAAGACTGGCTTAATGGAAAGGAGTATAAATGTAAGGTAA |
| | GTAACAAGGCATTGGCGGCTCCCATCGAGAAGACAATCTCCAAAGCAAAAGGGCAACCACGG |
| | GAGCCTCAGGTGTATACGTTGCCGCCCAGCAGAGATGAACTTACTAAGAATCAGGTGAGTCT |
| | CACTTGTCTCGTCAAGGGCTTCTATCCCAGCGATATAGCCGTAGAATGGGAGAGTAACGGTC |
| | AGCCGGAGAACAACTACAAAACAACCCCGCCTGTTTTGGACTCCGATGGGAGTTTTTTTCTC |
| | TACAGCAAACTCACGGTAGACAAAAGCAGGTGGCAGCAGGGCAATGTTTTCAGTTGCTCTGT |
| | TCTCCACGAAGCCCTCCACTCCCACTATACTCAGAAGTCTCTGAGTCTCTCACCAGGGGGAG |
| | GTAGCGATAGCTGGCAGGAGGAAGTGATCAAGTTGTGCGGGCGCGAACTCGTGCGGGCACAA |
| | ATTGCTATATGCGGTAAAAGTACGGGAGGTGGAGAGGAGGGTGGAGGTGAAGAAGGCGGTGG |
| | TAGACAATTGTATAGTGCGCTCGCCAACAAGTGTTGTCATGTCGGGTGTACGAAACGGTCCT |
| | TGGCGCGGTTTTGC |
| 143 | GACAAGACACATACTTGTCCACCATGTCCCGCCCCAGAAGCTGCGGGAGGACCATCAGTTTT |
| | TTTGTTCCCCCCGAAACCGAAGGATACCCTCATGATAAGTCGAACGCCCGAAGTCACTTGCG |
| | TGGTGGTTGATGTTAGCCACGAGGACCCAGAAGTGAAGTTCAACTGGTACGTGGACGGGGTC |
| | GAAGTTCATAATGCGAAAACAAAGCCTCGCGAGGAACAGTACAACTCTACATACAGGGTTGT |
| | GTCTGTTTTGACAGTCTTGCACCAAGATTGGCTCAACGGGAAGGAATATAAGTGTAAGGTAA |
| | GCAATAAAGCACTGGCGGCCCCGATCGAAAAAACGATATCCAAGGCCAAGGGCCAGCCCCGA |
| | GAGCCTCAGGTATATACTCTGCCGCCAAGCCGGGATGAACTGACTAAAAACCAGGTCTCTTT |
| | GACTTGTCTTGTCAAGGGATTTTACCCAAGTGACATTGCGGTAGAGTGGGAAAGCAACGGTC |
| | AACCAGAAAACAATTACAAGACGACACCGCCGGTACTCGACTCAGATGGATCCTTTTTCCTG |
| | TATAGCAAGCTGACAGTGGACAAGTCCCGGTGGCAGCAAGGGAACGTATTTTCATGCAGCGT |
| | GATGCATGAGGCTCTTCACAACCATTACACACAGAAAAGTCTGTCATTGAGCCCTGGCGGCG |
| | GGAGCGATTCTTGGCAAGAAGAAGTTATAAAACTTTGCGGTCGAGAGCTGGTTCGGGCACAA |
| | ATTGCTATCTGCGGAAAATCTACAGGAGGAGGCGAGGAGGGAGGGGGCGAAGAAGGCGGGGG |
| | GAGACAGTTGTACAGTGCGCTCGCTAACAAGTGTTGCCACGTCGGTTGCACAAAGAGATCCC |
| | TGGCTCAATTCTGT |
| 144 | GATAAAACTCACACCTGTCCCCCGTGTCCCGCACCAGAAGCGGCCGGTGGTCCCTCCGTTTT |
| | TCTCTTCCCTCCTAAACCTAAGGACACACTTATGATTAGCAGAACTCCAGAAGTTACGTGCG |
| | TAGTCGTTGACGTTAGTCATGAAGATCCTGAGGTTAAGTTCAACTGGTACGTAGACGGAGTA |
| | GAGGTCCACAACGCCAAGACGAAACCCCGAGAAGAGCAGTATAATTCTACCTATCGAGTTGT |
| | TTCAGTATTGACGGTGCTTCACCAAGATTGGCTGAATGGCAAAGAGTATAAGTGCAAGGTAA |
| | GCAACAAAGCACTCGCGGCTCCTATCGAGAAAACTATTTCCAAAGCTAAGGGCCAGCCTCGC |
| | GAACCACAAGTCTATACCCTGCCACCGAGTCGGGACGAACTCACCAAGAACCAAGTGTCTCT |
| | TACTTGCCTCGTTAAAGGTTTTTATCCCAGCGACATAGCCGTCGAATGGGAGTCCAATGGCC |
| | AACCTGAGAACAACTATAAAACTACCCCTCCTGTACTTGATAGCGACGGAAGTTTTTTCCTC |
| | TATTCAAAACTCACAGTTGATAAGTCTCGATGGCAACAGGGCAACGTCTTCTCTTGCAGTGT |
| | GTTGCATGAAGCTCTGCACTCTCATTACACACAGAAGAGTTTGTCTCTCAGTCCAGGTGGCG |
| | GCTCAGATAGCTGGCAGGAAGAAGTAATCAAGTTGTGCGGCAGGGAACTGGTAAGGGCACAG |
| | ATAGCCATTTGTGGAAAATCTACGGGTGGCGGTGAGGAAGGCGGCGGAGAAGAAGGGGGAGG |
| | TCGGCAGCTGTATAGTGCACTCGCAAACAAGTGCTGCCATGTCGGGTGCACCAAGCGATCCC |
| | TTGCCCAGTTTTGC |
| 145 | GACAAAACGCACACCTGTCCACCGTGTCCTGCTCCAGAGGCGGCCGGGGGACCGTCCGTTTT |
| | CCTTTTTCCTCCCAAACCTAAGGATACCCTTATGATCTCTCGCACGCCCGAGGTTACCTGTG |
| | TTGTGGTTGACGTGTCCCATGAAGACCCGGAAGTAAAATTTAATTGGTACGTGGACGGGGTC |
| | GAGGTTCATAACGCAAAGACCAAGCCACGAGAGGAGCAATATAATTCCACCTATCGCGTAGT |
| | CTCCGTCCTCACCGTGCTTCACCAGGATTGGCTCAACGGGAAGGAATACAAATGTAAAGTCA |
| | GTAATAAGGCTTTGGCGGCCCCGATTGAGAAGACTATAAGTAAGGCTAAGGGACAGCCACGA |
| | GAACCGCAAGTTTATACATTGCCCCCCTCTAGGGATGAGTTGACTAAGAATCAGGTGTCACT |
| | CACTTGTCTGGTAAAAGGGTTCTACCCGTCCGACATCGCTGTGGAATGGGAAAGCAATGGGC |
| | AACCTGAAAATAATTATAAGACAACCCCTCCGGTGCTTGATAGCGACGGATCATTCTTTCTC |
| | TATTCCAAGCTTACTGTAGATAAGAGTCGATGGCAACAGGGGAACGTATTCAGTTGCTCTGT |
| | TCTCCATGAGGCCCTGCATAGTCACTACACCCAAAAAAGCCTTAGTTTGAGTCCCGGGAAAG |
| | GAGGCTCCGATTCTTGGCAAGAAGAGGTAATAAAGCTGTGTGGACGAGAACTTGTCCGAGCA |
| | CAAATTGCGATTTGTGGCAAATCTACAGGAGGGGGAGAAGGAGGCGGCGAAGGGGGAGGCGA |
| | GGGCAGGCAGGATTATTCCGCTCTGGCGAACAAATGTTGCCATGTTGGATGCACGAAACGAA |
| | GCCTGGCTCAGTTTTGC |
| 146 | GATAAAACGCATACCTGCCCACCGTGTCCTGCACCTGAGGCCGCTGGAGGACCTTCCGTCTT |
| | CCTCTTTCCACCCAAGCCGAAAGACACACTCATGATTAGTAGAACTCCAGAGGTCACGTGTG |
| | TTGTGGTGGACGTCAGTCACGAGGACCCTGAGGTTAAGTTCAACTGGTACGTTGATGGCGTA |
| | GAAGTCCACAATGCAAAGACCAAACCGAGAGAGGAGCAATATAACAGTACATATAGGGTTGT |
| | TAGCGTACTTACTGTTTTGCATCAAGACTGGTTGAATGGGAAGGAATATAAATGTAAAGTCT |
| | CCAACAAGGCTCTGGCTGCACCAATAGAAAAAACTATTTCTAAGGCAAAGGGTCAGCCTAGA |
| | GAGCCTCAAGTCTATACCTTGCCACCGTCAAGAGACGAGCTCACTAAAAATCAGGTGAGCCT |
| | GACCTGTCTTGTGAAGGGCTTTTACCCGTCAGATATTGCCGTGGAGTGGGAATCAAACGGTC |
| | AGCCGGAGAATAACTACAAGACGACCCCACCAGTACTCGATAGCGATGGGTCTTTCTTTCTG |
| | TACTCCAAGCTCACCGTGGACAAATCACGCTGGCAACAGGGCAACGTCTTTAGTTGCAGCGT |
| | ACTGCACGAGGCACTGCACAGCCACTACACACAAAAGAGTCTTTCTCTGTCTCCCGGTGGTG |
| | GCTCCGATAGTTGGCAGGAAGAAGTCATAAAGCTTTGTGGAAGAGAGCTTGTACGAGCGCAG |
| | ATTGCAATCTGCGGGAAGAGCACTGGAGGAGGTGAGGGAGGGGGTGAGGGCGGGGGCGAAGG |
| | ACGCCAGGACTATTCAGCACTTGCAAACAAATGCTGCCATGTAGGGTGTACGAAGCGCTCAC |
| | TGGCCCGGTTTTGC |
| 147 | GATAAAACACATACCTGCCCCCCATGCCCAGCCCCCGAAGCTGCAGGGGGGCCCTCTGTTTT |
| | CCTTTTTCCACCCAAACCTAAAGATACTCTGATGATTAGTCGGACTCCGGAAGTGACTTGCG |
| | TCGTTGTCGACGTCTCTCATGAGGATCCAGAAGTTAAGTTTAACTGGTATGTCGACGGGGTT |
| | GAGGTTCATAATGCAAAAACTAAACCGAGAGAAGAACAGTACAACTCTACTTATAGGGTTGT |
| | CAGTGTACTGACCGTCCTGCACCAGGATTGGCTTAACGGTAAGGAGTATAAGTGTAAAGTGT |
| | CCAATAAAGCCCTTGCCGCACCCATCGAGAAAACCATCTCCAAGGCAAAAGGACAGCCAAGG |
| | GAACCGCAGGTATATACACTTCCGCCAAGCCGAGACGAACTTACGAAGAACCAGGTGTCTCT |
| | CACGTGTCTCGTAAAAGGGTTTTATCCCAGCGATATCGCAGTTGAGTGGGAGAGCAATGGGC |
| | AGCCAGAGAATAATTATAAGACAACCCCTCCCGTGCTGGATTCAGACGGGAGTTTTTTTCTT |
| | TACTCTAAGCTGACCGTAGACAAAAGTCGATGGCAGCAAGGCAACGTCTTTTCCTGCTCCGT |
| | TCTCCATGAAGCACTGCATAGCCATTATACCCAGAAGTCACTGAGCCTCTCTCCAGGGGGCG |
| | GGTCCGATTCATGGCAGGAAGAGGTAATCAAACTCTGTGGACGCGAACTGGTTCGCGCGCAG |
| | ATAGCGATTTGCGGCAAAAGCACAGGCGGTGGGGAAGGCGGTGGCGAGGGCGGTGGTGAAGG |
| | TCGACAAGATTATTCTGCTCTCGCTAACAAGTGTTGTCATGTAGGATGTACTAAAAGGAGTC |
| | TTGCGCAGTTCTGT |
| 148 | GATAAGACGCACACATGCCCACCCTGTCCTGCGCCTGAAGCCGCGGGGGGACCCAGCGTTTT |
| | TCTCTTCCCGCCGAAACCGAAAGACACACTTATGATCAGCCGGACTCCCGAGGTTACCTGCG |
| | TGGTGGTAGATGTATCTCACGAGGATCCCGAGGTCAAATTCAACTGGTACGTTGATGGGGTT |
| | GAAGTTCATAATGCCAAAACGAAGCCAAGAGAAGAGCAGTATAACTCCACATATAGAGTTGT |
| | TTCCGTCTTGACTGTTCTTCACCAAGATTGGCTGAATGGGAAGGAGTACAAATGTAAAGTTA |
| | GCAACAAGGCACTCGCCGCTCCCATTGAAAAAACTATAAGCAAAGCTAAGGGCCAACCGCGC |
| | GAACCACAGGTCTACACGTTGCCGCCCTCTAGGGACGAACTCACGAAGAATCAGGTTTCCCT |
| | TACCTGCCTCGTTAAAGGATTCTACCCCTCTGACATAGCGGTTGAATGGGAGAGCAACGGTC |
| | AGCCTGAGAACAACTACAAAACGACGCCTCCGGTGTTGGATTCCGACGGTAGTTTTTTCCTC |
| | TATAGTAAGCTGACAGTGGATAAATCTCGGTGGCAGCAAGGGAATGTATTCTCCTGTTCAGT |
| | CCTGCATGAAGCCCTCCACTCCCATTATACACAGAAATCTCTTTCTCTGAGTCCCGGTAAAG |
| | GTGGGAGTGACTCTTGGCAGGAAGAGGTAATTAAGTTGTGTGGAAGGGAGCTGGTAAGAGCA |
| | CAGATTGCCATCTGTGGCAAATCCACGGGCGGCGAAGGTGAGGGGGGTGAGGGGGAAGGGGG |
| | GTCCAGACAACTGTATTCTGCTCTGGCGAATAAGTGTTGCCATGTAGGGTGCACTAAACGGT |
| | CCTTGGCGCAGTTCTGT |
| 149 | GATAAAACTCATACGTGCCCACCTTGCCCCGCACCGGAGGCTGCTGGAGGACCCTCTGTCTT |
| | CCTGTTCCCGCCGAAGCCTAAAGACACATTGATGATCAGTCGAACACCGGAAGTCACCTGTG |
| | TAGTGGTTGATGTGAGCCATGAGGACCCTGAAGTAAAATTTAACTGGTATGTTGATGGCGTA |
| | GAAGTACACAACGCGAAGACTAAACCAAGGGAAGAGCAATACAACTCTACCTATAGGGTCGT |
| | TAGCGTACTGACTGTGCTTCACCAAGACTGGCTTAACGGGAAGGAGTACAAGTGCAAAGTGA |
| | GCAATAAGGCCCTCGCCGCGCCTATCGAGAAAACCATTTCCAAAGCCAAGGGTCAACCAAGG |
| | GAGCCTCAGGTTTACACCCTGCCCCCTTCAAGGGATGAGTTGACAAAAAACCAGGTAAGTCT |
| | GACGTGTCTCGTTAAGGGATTCTACCCGTCAGATATCGCGGTAGAGTGGGAGAGCAACGGTC |
| | AGCCAGAAAATAATTACAAAACAACACCTCCAGTTTTGGACTCTGATGGGAGTTTTTTTCTT |
| | TATTCTAAGTTGACAGTGGATAAGTCACGCTGGCAACAGGGGAACGTATTTAGCTGCTCAGT |
| | ACTTCATGAAGCGTTGCATTCTCACTACACACAGAAGAGCCTCTCCTTGAGTCCCGGAGGTG |
| | GCTCTGATTCTTGGCAGGAGGAGGTAATAAAACTTTGTGGTAGAGAACTGGTTCGCGCTCAG |
| | ATAGCTATTTGTGGAAAATCCACTGGCGGTGAAGGTGAAGGTGGAGAAGGAGAGGGCGGAAG |
| | CCGGCAGTTGTACTCTGCCCTGGCTAATAAGTGCTGTCACGTGGGCTGCACTAAGCGGAGCT |
| | TGGCAAGATTTTGC |
| 150 | GATAAAACTCATACCTGTCCACCTTGTCCTGCGCCTGAGGCAGCTGGAGGGCCTAGCGTGTT |
| | CCTGTTCCCCCCCAAACCCAAAGACACGCTCATGATTAGCCGAACCCCTGAAGTGACCTGCG |
| | TTGTTGTGGACGTAAGCCACGAAGACCCCGAAGTTAAGTTTAATTGGTACGTCGACGGTGTT |
| | GAGGTTCATAACGCGAAGACTAAGCCGAGAGAGGAGCAATATAACAGCACCTACCGCGTAGT |
| | CTCAGTTCTTACCGTGCTCCACCAGGACTGGCTTAACGGGAAGGAATACAAATGCAAAGTTT |
| | CCAACAAAGCCTTGGCAGCCCCAATAGAGAAGACAATATCTAAGGCGAAAGGCCAACCGCGG |
| | GAACCGCAAGTTTATACCCTCCCACCGAGCAGGGATGAGCTGACAAAAAATCAGGTTTCCCT |
| | CACTTGTCTGGTCAAGGGATTTTATCCTTCAGACATAGCCGTTGAATGGGAGAGTAATGGGC |
| | AGCCGGAGAATAATTACAAGACCACCCCCCCGGTGTTGGACAGCGACGGTTCCTTCTTTCTC |
| | TATTCTAAACTTACCGTCGACAAATCACGGTGGCAACAAGGAAATGTATTCTCATGCAGTGT |
| | ATTGCACGAAGCTCTGCACTCTCATTACACCCAAAAATCCCTCTCTCTCAGCCCTGGCGGTG |
| | GATCTGATTCTTGGCAGGAAGAGGTGATTAAACTGTGTGGGCGAGAGCTTGTCCGAGCTCAG |
| | ATCGCTATTTGTGGCAAGAGTACCGGAGGCGAGGGTGAGGGAGGCGAAGGCGAGGGCGGAAG |
| | CCGGCAACTCTATAGCGCACTCGCTAATAAATGTTGTCATGTCGGCTGCACGAAGCGCTCAC |
| | TGGCGCAGTTCTGC |
| 151 | GACAAAACGCATACCTGTCCTCCATGCCCCGCTCCCGAGGCTGCCGGCGGACCAAGCGTATT |
| | TCTCTTCCCCCCTAAACCTAAAGACACATTGATGATAAGTAGGACGCCTGAAGTAACGTGTG |
| | TTGTCGTTGATGTAAGCCATGAAGATCCTGAAGTAAAGTTTAATTGGTATGTTGATGGCGTA |
| | GAAGTACATAACGCTAAGACGAAGCCACGGGAAGAGCAGTATAACTCAACTTACCGCGTTGT |
| | AAGCGTGCTTACCGTCCTGCATCAGGATTGGCTGAATGGTAAGGAATATAAGTGCAAAGTAA |
| | GCAACAAAGCATTGGCCGCACCAATAGAGAAGACGATTAGTAAAGCAAAAGGCCAGCCCAGA |
| | GAGCCGCAGGTTTATACACTTCCACCAAGCAGAGATGAACTTACGAAGAACCAGGTGTCTCT |
| | GACTTGTCTGGTCAAGGGTTTCTATCCTTCCGACATTGCAGTGGAGTGGGAAAGCAATGGGC |
| | AGCCCGAAAACAATTATAAGACGACACCTCCAGTGTTGGACTCAGACGGTTCCTTTTTCTTG |
| | TATTCCAAACTTACAGTGGATAAGTCAAGGTGGCAGCAAGGCAACGTATTTTCTTGTAGTGT |
| | TTTGCACGAAGCCCTGCATTCCCACTATACTCAAAAGAGCCTCAGTCTGTCCCCAGGAAAGG |
| | GAGGGAGTGACAGTTGGCAAGAGGAGGTAATAAAATTGTGTGGCAGAGAGCTTGTGCGCGCT |
| | CAGATCGCAATATGCGGGAAATCTACTGGGGGTGAGGGTGAGGGCGGCGAGGGAGAGGGGGG |
| | CAGTCGCCAAGATTATTCCGCCCTTGCGAATAAGTGTTGTCACGTCGGATGTACTAAGAGAT |
| | CATTGGCTCAGTTTTGT |
| 152 | ATGGAGACGGACACTTTGCTGCTTTGGGTACTGCTGCTTTGGGTTCCTGGATCTACTGGCGA |
| | TAAAACACACACGTGTCCCCCCTGCCCGGCTCCAGAGGCGGCTGGTGGTCCCAGCGTATTCT |
| | TGTTTCCTCCCAAACCTAAGGATACGCTCATGATATCCCGCACCCCAGAAGTTACGTGTGTA |
| | GTCGTCGACGTCAGTCACGAAGATCCAGAGGTCAAATTTAACTGGTATGTCGACGGAGTAGA |
| | GGTCCACAATGCGAAAACCAAGCCCAGAGAAGAGCAGTACAACTCCACGTATCGCGTCGTCT |
| | CCGTCCTCACCGTACTCCATCAAGATTGGCTGAATGGGAAAGAGTATAAATGCAAAGTATCT |
| | AACAAGGCTCTGCCAGCTCCGATAGAAAAGACTATATCAAAGGCCAAGGGGCAGCCAAGGGA |
| | GCCTCAAGTCTATACTTTGCCCCCATCTCGGGATGAGCTTACGAAAAACCAGGTCAGCCTTA |
| | CCTGTCTTGTTAAAGGTTTTTATCCGAGTGACATCGCAGTGGAATGGGAATCTAATGGTCAA |
| | CCTGAAAACAATTACAAAACCACACCGCCAGTATTGGACAGCGATGGTAGTTTTTTTCTTTA |
| | CTCAAAACTGACTGTAGATAAAAGCAGATGGCAGCAGGGCAATGTCTTTTCATGTAGCGTTA |
| | TGCATGAGGCTCTTCACAACCACTATACCCAAAAGTCATTGTCTCTTAGTCCCGGAAAGGGC |
| | GGAAGTGATTCTTGGAAGGAGGAGGTAATCAAGTTGTGCGGGCGAGAGTTGGTACGGGCACA |
| | GATCGCGATATGCGGAAAATCCACAGGTGGGGGCGAAGGAGGAGGTGAGGGTGGAGGTGAAG |
| | GACGACAGTTGTATTCCGCCTTGGCAAACAAGTGTTGCCATGTGGGTTGCACAAAACGCAGT |
| | CTTGCCCGCTTCTGT |
| 153 | ATGGAGACCGATACGCTGTTGCTGTGGGTATTGCTTCTCTGGGTGCCCGGCTCAACTGGGGA |
| | TAAGACACATACATGCCCTCCCTGTCCGGCTCCAGAGGCAGCCGGGGGTCCATCAGTCTTCC |
| | TTTTTCCGCCTAAACCTAAGGATACACTGATGATCTCTCGAACACCGGAGGTCACTTGTGTT |
| | GTCGTTGACGTATCACATGAGGATCCCGAAGTAAAGTTCAACTGGTATGTCGATGGTGTGGA |
| | GGTTCATAATGCTAAAACTAAACCACGGGAGGAGCAATATAATTCCACATATAGGGTCGTGA |
| | GCGTGTTGACGGTGCTTCATCAAGACTGGCTTAATGGGAAGGAATATAAATGCAAAGTGTCA |
| | AATAAAGCACTTCCTGCGCCAATCGAGAAAACAATTAGTAAGGCAAAGGGGCAGCCGCGAGA |
| | ACCTCAGGTGTACACCTTGCCGCCTTCTAGAGACGAGCTCACAAAGAACCAAGTTTCCCTGA |
| | CTTGCCTCGTTAAGGGGTTTTATCCGTCCGATATAGCCGTGGAGTGGGAGTCAAACGGCCAA |
| | CCGGAAAATAATTACAAAACGACACCCCCAGTATTGGATAGTGACGGCTCTTTTTTCCTTTA |
| | TTCTAAGCTGACTGTGGACAAAAGCCGCTGGCAGCAGGGCAATGTCTTTTCATGCAGCGTAA |
| | TGCATGAAGCCCTGCACAACCACTACACGCAAAAATCCCTTTCCTTGTCACCCGGCAAGGGC |
| | GGCTCTGACTCCTGGAAAGAGGAAGTTATAAAACTCTGTGGCCGAGAACTTGTTCGAGCTCA |
| | AATCGCGATTTGTGGTAAGTCAACGGGTGGGGGCGAAGGTGGAGGCGAGGGTGGGGGAGAAG |
| | GAGGAGGCCAGTTGTACTCAGCTCTTGCAAATAAGTGTTGCCACGTTGGTTGTACGAAGCGG |
| | AGCCTTGCTCGCTTCTGC |
| 154 | ATGGAAACTGATACTCTTCTGCTGTGGGTCCTGCTGCTGTGGGTTCCAGGATCTACTGGAGA |
| | CAAAACACATACTTGTCCGCCTTGCCCGGCACCCGAAGCGGCCGGCGGACCCAGTGTCTTTC |
| | TCTTCCCACCCAAACCGAAAGACACTCTGATGATTTCCAGGACGCCTGAAGTGACCTGCGTT |
| | GTAGTTGATGTATCACACGAGGATCCCGAGGTCAAGTTCAATTGGTATGTAGATGGGGTGGA |
| | GGTCCATAATGCAAAGACGAAGCCACGGGAGGAACAGTACAACTCTACGTACAGAGTTGTCA |
| | GTGTTTTGACCGTCCTTCATCAGGATTGGCTGAACGGTAAAGAATATAAATGCAAGGTTAGC |
| | AATAAAGCTTTGCCCGCCCCTATAGAGAAAACGATCAGTAAGGCGAAGGGGCAGCCTAGGGA |
| | ACCCCAGGTATATACCTTGCCGCCAAGTCGAGATGAGCTGACGAAGAACCAAGTGAGTCTGA |
| | CATGCCTCGTGAAGGGCTTCTATCCGAGCGATATCGCTGTCGAATGGGAGAGCAATGGGCAG |
| | CCTGAGAATAACTATAAAACAACGCCACCCGTCCTCGACTCCGATGGCTCATTCTTCCTGTA |
| | CAGTAAACTTACAGTAGATAAGAGTAGATGGCAGCAGGGTAACGTCTTTAGTTGCTCCGTGA |
| | TGCACGAGGCATTGCACAATCATTACACTCAAAAATCTCTGTCCCTGAGTCCGGGCAAAGGC |
| | GGTTCAGATAGCTGGATGGAGGAGGTCATAAAGCTTTGTGGACGAGAACTCGTTCGCGCCCA |
| | GATAGCTATTTGTGGGAAATCAACCGGGGGTGGAGAAGGTGGCGGAGAAGGGGGAGGCGAAG |
| | GGCGCCAACTGTATTCTGCATTGGCTAATAAGTGCTGTCACGTAGGATGTACAAAAAGGTCT |
| | CTGGCGAGATTCTGC |
| 155 | ATGGAGACCGACACCCTCTTGTTGTGGGTTCTCCTCTTGTGGGTGCCCGGCAGTACTGGAGA |
| | CAAGACGCACACTTGTCCACCTTGCCCTGCGCCGGAAGCTGCTGGAGGCCCCAGTGTCTTTT |
| | TGTTCCCGCCCAAACCGAAGGACACTTTGATGATAAGTCGCACGCCCGAGGTTACCTGTGTG |
| | GTTGTCGATGTCTCACACGAAGATCCGGAGGTGAAGTTTAATTGGTATGTAGATGGCGTGGA |
| | GGTTCATAACGCCAAAACGAAACCCAGAGAAGAACAATATAACAGTACATATCGAGTAGTAT |
| | CCGTTCTCACTGTCCTGCATCAAGACTGGTTGAACGGGAAGGAATATAAGTGCAAGGTGAGC |
| | AATAAAGCACTCCCGGCCCCAATCGAAAAGACCATCAGCAAAGCGAAGGGGCAACCTCGAGA |
| | ACCCCAGGTATATACGCTCCCCCCTAGTCGGGATGAACTTACTAAAAATCAGGTTAGCCTCA |
| | CTTGCCTTGTTAAAGGGTTCTATCCCAGTGATATTGCCGTCGAATGGGAATCAAACGGGCAG |
| | CCGGAAAATAACTACAAGACAACCCCTCCTGTGCTCGATAGCGATGGCTCTTTTTTCCTCTA |
| | CAGCAAACTTACCGTTGATAAGAGCCGGTGGCAACAAGGTAATGTTTTCTCCTGCTCCGTTA |
| | TGCATGAAGCACTCCATAACCATTATACCCAAAAAAGCCTGTCACTTAGTCCGGGTAAAGGA |
| | GGTAGTGATTCTTGGCAGGAGGAGGTAATCAAACTTTGTGGGAGGGAGCTGGTACGAGCTCA |
| | GATTGCTATATGTGGAAAAAGCACGGGCGGAGGAGAAGGAGGTGGCGAAGGCGGGGGTGAAG |
| | GTCGGCAACTCTACTCCGCTCTCGCTAATAAGTGCTGCCACGTCGGGTGTACGAAGCGCTCC |
| | CTGGCGCGATTCTGC |
| 156 | ATGGAAACAGATACCCTCCTCCTCTGGGTCCTTCTTCTTTGGGTGCCTGGCTCAACTGGAGA |
| | TAAAACGCACACGTGTCCGCCCTGCCCAGCGCCTGAAGCCGCAGGCGGGCCGTCCGTCTTCC |
| | TCTTTCCTCCAAAACCCAAAGACACACTTATGATCAGTAGGACCCCAGAGGTAACCTGCGTC |
| | GTGGTCGACGTTTCCCATGAAGACCCAGAGGTCAAGTTCAACTGGTACGTCGACGGTGTCGA |
| | AGTACATAATGCTAAAACGAAGCCTCGGGAAGAGCAGTACAACTCTACCTACCGCGTCGTTT |
| | CCGTACTCACCGTACTTCACCAGGACTGGCTTAACGGTAAAGAGTATAAATGCAAAGTATCT |
| | AATAAGGCTCTCGCCGCGCCGATTGAGAAGACAATTTCAAAGGCCAAGGGGCAGCCGCGGGA |
| | GCCCCAAGTGTATACCTTGCCCCCGTCCCGAGATGAGCTGACTAAAAACCAAGTAAGCTTGA |
| | CTTGCTTGGTCAAAGGCTTCTACCCTTCCGATATAGCTGTCGAATGGGAGTCAAATGGCCAA |
| | CCAGAGAACAATTATAAAACTACACCCCCGGTCTTGGATTCTGATGGCTCATTTTTTCTCTA |
| | TTCTAAACTGACCGTGGATAAGTCTCGCTGGCAGCAAGGTAACGTGTTCAGTTGCTCTGTTC |
| | TTCACGAAGCACTGCACAGTCATTACACTCAGAAGAGTCTTAGCCTGAGCCCTGGTAAAGGG |
| | GGTTCTGATTCCTGGCAGGAGGAAGTAATAAAACTCTGTGGCCGGGAGTTGGTACGGGCGCA |
| | GATTGCGATATGCGGTAAGAGCACCGGCGGAGGCGAAGGCGGTGGGGAAGGAGGAGGAGAAG |
| | GGAGACAACTCTATTCCGCATTGGCAAATAAGTGCTGCCACGTCGGGTGTACCAAACGATCC |
| | CTTGCACGGTTCTGT |
| 157 | ATGGAGACGGACACCCTCCTTCTCTGGGTTTTGCTCCTTTGGGTCCCTGGTTCCACTGGAGA |
| | TAAGACCCATACGTGCCCCCCTTGCCCTGCGCCTGAGGCAGCGGGTGGCCCATCAGTCTTTT |
| | TGTTCCCGCCCAAGCCAAAGGACACCCTCATGATTAGTAGAACACCGGAGGTTACGTGCGTC |
| | GTAGTGGATGTCAGCCACGAGGATCCCGAGGTTAAGTTTAACTGGTACGTTGATGGGGTTGA |
| | GGTCCATAATGCGAAGACTAAGCCGAGAGAGGAACAGTACAATTCCACGTATAGAGTTGTCT |
| | CTGTACTGACTGTGCTGCATCAAGATTGGCTTAACGGTAAGGAGTACAAGTGCAAAGTCTCT |
| | AATAAGGCTCTTCCTGCACCCATTGAGAAAACTATAAGCAAAGCAAAAGGTCAACCTCGCGA |
| | ACCTCAGGTGTACACACTGCCACCCTCTAGGGACGAGCTTACCAAAAATCAAGTATCTCTTA |
| | CCTGCCTTGTGAAAGGGTTTTATCCCTCAGATATTGCGGTTGAGTGGGAGTCTAACGGACAA |
| | CCTGAGAACAACTATAAGACTACTCCCCCGGTGCTTGATTCAGACGGGAGTTTTTTTTTGTA |
| | TAGCAAACTTACCGTCGACAAAAGCCGGTGGCAACAGGGCAATGTATTCAGTTGTTCTGTAA |
| | TGCATGAAGCTTTGCATAATCATTACACCCAAAAGAGTCTTTCCCTGTCTCCTGGAAAAGGG |
| | GGGTCAGACTCCTGGATGGAGGAGGTGATCAAACTGTGTGGGAGAGAGCTCGTCCGGGCTCA |
| | GATAGCTATATGCGGCAAGTCTACGGGTGGGGGAGAGGGCGGAGGAGAGGGCGGTGGAGAAG |
| | GAGGCGGCCAACTCTACAGCGCTCTGGCCAATAAATGTTGTCATGTCGGGTGTACTAAGCGC |
| | TCACTGGCACGCTTTTGC |
| 158 | ATGGAAACCGACACCCTTTTGTTGTGGGTATTGCTGTTGTGGGTTCCCGGTAGCACGGGGGA |
| | CAAGACGCATACATGCCCGCCATGCCCGGCCCCCGAAGCTGCTGGGGGACCATCCGTATTCC |
| | TCTTCCCTCCCAAACCAAAAGACACGTTGATGATAAGTAGAACACCAGAGGTAACGTGCGTG |
| | GTTGTCGATGTTTCCCACGAAGATCCGGAGGTAAAATTCAATTGGTATGTAGATGGGGTGGA |
| | AGTGCACAATGCCAAAACAAAGCCGCGAGAAGAACAATACAATAGTACTTACCGGGTTGTGA |
| | GCGTGCTCACGGTGTTGCACCAAGACTGGCTCAACGGCAAGGAATACAAGTGCAAAGTATCT |
| | AATAAAGCTCTGCCTGCGCCGATAGAGAAGACCATCAGTAAGGCCAAAGGGCAGCCCCGAGA |
| | GCCGCAAGTTTACACTCTTCCTCCGAGCAGAGATGAATTGACCAAGAACCAAGTAAGTTTGA |
| | CGTGCCTGGTGAAGGGCTTCTACCCCTCAGACATTGCGGTGGAGTGGGAAAGTAATGGTCAA |
| | CCGGAAAACAACTACAAGACCACGCCGCCCGTCCTCGACTCCGATGGGTCTTTCTTTCTTTA |
| | TTCAAAGTTGACAGTAGATAAGTCAAGGTGGCAGCAAGGTAACGTGTTTAGTTGTAGTGTAA |
| | TGCACGAGGCCCTGCATAATCATTATACCCAAAAGAGTTTGAGCCTCTCACCAGGAAAAGGC |
| | GGATCAGACAGCTGGCAGGAGGAGGTAATTAAATTGTGTGGACGGGAGTTGGTCAGGGCGCA |
| | AATAGCCATCTGCGGTAAGAGCACGGGTGGAGGAGAGGGTGGAGGGGAAGGTGGGGGAGAAG |
| | GCGGCGGGCAGCTCTATTCTGCACTCGCCAACAAGTGTTGTCACGTCGGATGCACAAAGAGA |
| | TCTCTTGCTCGATTCTGC |
| 159 | ATGGAGACTGATACTCTTTTGTTGTGGGTACTGCTCCTGTGGGTTCCAGGCTCCACAGGAGA |
| | CAAAACACACACCTGTCCGCCTTGCCCGGCTCCTGAAGCCGCGGGTGGCCCTAGTGTGTTTT |
| | TGTTTCCGCCGAAACCTAAGGATACCCTCATGATAAGCCGGACGCCCGAGGTTACCTGTGTC |
| | GTGGTCGATGTTAGTCATGAGGATCCAGAAGTCAAGTTTAATTGGTACGTCGACGGCGTTGA |
| | AGTCCACAATGCAAAAACTAAACCGCGAGAAGAACAGTACAACTCCACCTACAGAGTTGTCT |
| | CAGTTTTGACAGTTCTCCATCAGGATTGGCTCAATGGAAAGGAATATAAGTGCAAGGTCAGC |
| | AATAAAGCGCTTGCCGCCCCTATAGAGAAGACCATTAGCAAGGCGAAAGGACAGCCCCGCGA |
| | GCCCCAGGTCTATACGCTGCCTCCTAGCAGAGATGAGCTCACGAAAAATCAGGTCAGCTTGA |
| | CATGCTTGGTGAAGGGCTTCTACCCCAGTGACATCGCAGTTGAATGGGAGAGCAACGGCCAA |
| | CCTGAGAACAACTACAAAACAACGCCCCCGGTTCTTGACAGCGATGGGTCCTTCTTTCTTTA |
| | CTCTAAGCTTACAGTTGATAAAAGCAGGTGGCAGCAGGGGAATGTGTTCTCATGTTCCGTAC |
| | TGCATGAGGCTCTGCATTCTCACTACACCCAAAAAAGCCTTAGCCTGAGCCCCGGTAAGGGA |
| | GGTAGTGACTCATGGCAAGAGGAAGTGATTAAGCTCTGCGGCCGGGAGTTGGTGAGAGCCCA |
| | AATCGCCATTTGCGGTAAAAGTACCGGAGGGGGCGAGGGAGGAGGCGAAGGTGGAGGTGAAG |
| | GAGGTGGACAGTTGTACTCAGCTCTTGCAAATAAATGTTGTCATGTTGGTTGCACGAAAAGA |
| | TCTCTTGCGAGGTTCTGT |
| 160 | ATGGAGACTGACACTCTTTTGTTGTGGGTGCTTCTTCTGTGGGTACCTGGATCCACTGGGGA |
| | TAAGACGCATACTTGTCCACCGTGCCCCGCACCGGAAGCGGCTGGTGGTCCATCAGTTTTTC |
| | TGTTCCCACCGAAACCTAAGGACACGTTGATGATATCACGGACACCAGAGGTTACGTGCGTA |
| | GTGGTGGATGTGAGCCACGAGGATCCAGAAGTTAAATTTAATTGGTACGTAGATGGAGTGGA |
| | GGTTCATAATGCGAAGACAAAGCCTCGCGAGGAACAGTATAATTCCACCTATCGCGTCGTAT |
| | CTGTGCTTACGGTACTTCACCAAGACTGGTTGAACGGTAAGGAATATAAATGCAAGGTTTCC |
| | AATAAAGCACTTCCTGCGCCAATTGAGAAGACAATATCCAAAGCTAAAGGTCAACCCAGGGA |
| | ACCGCAAGTCTACACTCTCCCCCCGTCTCGCGATGAATTGACGAAGAACCAGGTTAGTCTCA |
| | CCTGCCTGGTCAAGGGGTTTTACCCCTCTGACATAGCTGTAGAATGGGAGTCTAATGGACAG |
| | CCAGAGAACAATTACAAAACGACCCCCCCGGTCCTCGATTCTGATGGGAGTTTTTTTCTTTA |
| | TTCAAAATTGACTGTCGATAAGTCAAGATGGCAACAGGGTAACGTATTTTCTTGCAGTGTTA |
| | TGCATGAAGCATTGCACAACCACTATACACAAAAATCATTGAGTTTGAGTCCCGGTAAAGGG |
| | GGAAGCGACTCATGGATGGAAGAAGTAATCAAGCTGTGCGGGCGAGAGCTTGTGCGAGCTCA |
| | GATAGCAATCTGTGGTAAGTCTACAGGTGGAGAGGGTGGCGGTGAAGAAGGCGGGGGAGAGG |
| | GAGGCCAGCTTTATTCTGCCCTGGCTAACAAGTGCTGTCACGTTGGATGCACGAAGCGCTCC |
| | CTGGCCCGATTCTGC |
| 161 | ATGGAAACCGATACATTGCTTTTGTGGGTCCTCCTTCTTTGGGTTCCTGGCTCTACAGGCGA |
| | TAAGACGCATACTTGTCCCCCATGTCCCGCTCCGGAAGCCGCTGGCGGCCCCTCCGTTTTTC |
| | TGTTCCCGCCGAAACCGAAAGACACCCTGATGATATCACGCACTCCCGAGGTCACTTGCGTG |
| | GTAGTCGATGTTAGTCATGAAGATCCTGAGGTCAAATTCAATTGGTATGTAGATGGCGTTGA |
| | GGTACACAACGCGAAGACAAAACCCCGAGAAGAACAGTATAACTCAACCTACCGCGTAGTTT |
| | CAGTTCTTACCGTACTGCACCAAGACTGGTTGAACGGTAAAGAGTACAAATGTAAAGTCAGC |
| | AATAAAGCTTTGCCAGCACCTATCGAAAAAACCATCAGTAAGGCCAAGGGTCAACCCAGGGA |
| | GCCGCAAGTGTACACTCTTCCCCCTAGCAGGGATGAATTGACCAAGAATCAGGTCTCTTTGA |
| | CGTGCCTCGTTAAGGGTTTCTATCCCAGCGATATAGCCGTAGAATGGGAGTCTAACGGTCAG |
| | CCAGAAAATAACTATAAGACAACCCCGCCTGTTTTGGATTCCGACGGCTCTTTTTTTCTCTA |
| | CTCTAAGTTGACCGTTGATAAGAGCAGATGGCAGCAGGGAAACGTATTTTCTTGTTCCGTGA |
| | TGCACGAAGCCCTGCACAATCACTATACGCAAAAGTCTCTGAGCTTGAGTCCGGGTAAAGGC |
| | GGTTCTGACTCCTGGCAGGAGGAAGTCATAAAACTCTGCGGAAGAGAGCTCGTAAGGGCGCA |
| | AATCGCTATTTGTGGTAAGAGCACCGGTGGGGAAGGAGGCGGTGAAGAGGGTGGCGGCGAGG |
| | GTGGGCAATTGTATTCCGCGCTTGCCAATAAATGTTGTCACGTAGGCTGCACAAAGCGAAGT |
| | CTCGCTAGGTTCTGC |
| 162 | ATGGAAACCGACACCTTGCTTTTGTGGGTGCTCTTGCTGTGGGTTCCGGGGAGCACTGGCGA |
| | CAAGACCCACACATGTCCCCCGTGTCCGGCACCAGAAGCAGCGGGGGGACCGTCAGTATTCT |
| | TGTTTCCACCGAAGCCCAAAGACACATTGATGATTTCACGAACTCCTGAAGTTACCTGTGTG |
| | GTTGTAGATGTATCACACGAAGACCCAGAAGTCAAATTCAATTGGTATGTCGACGGGGTTGA |
| | AGTTCACAATGCGAAGACGAAGCCCCGGGAGGAACAGTACAACAGCACGTACAGGGTTGTGA |
| | GCGTTCTTACTGTATTGCACCAGGATTGGCTCAACGGCAAGGAGTATAAATGTAAAGTTTCT |
| | AATAAGGCTCTTCCTGCCCCAATTGAAAAGACGATATCTAAAGCGAAGGGCCAACCACGGGA |
| | ACCTCAGGTGTACACACTTCCGCCTAGCAGGGATGAGTTGACCAAGAATCAAGTCTCTTTGA |
| | CGTGCCTGGTCAAGGGGTTTTACCCATCAGATATCGCCGTCGAATGGGAGTCAAACGGACAA |
| | CCCGAAAATAACTATAAAACTACTCCACCAGTTCTGGATAGCGACGGCTCATTTTTTCTGTA |
| | TTCAAAGCTCACTGTAGACAAGTCTAGGTGGCAGCAGGGTAATGTCTTCTCCTGCTCAGTAA |
| | TGCATGAGGCTCTTCACAACCACTATACTCAAAAGAGCCTTTCCCTGTCACCTGGCGGTGGA |
| | AGCGACTCATGGATGGAGGAGGTAATAAAGCTCTGCGGAAGAGAACTGGTACGCGCACAAAT |
| | CGCAATTTGTGGTAAGAGTACTGGCGGGGAAGGAGGTGGGGAAGAAGGGGGCGGTGAGGGCG |
| | GACAGCTCTATTCTGCACTTGCAAACAAATGTTGCCACGTGGGATGTACTAAGCGAAGCCTT |
| | GCAAGATTCTGC |
| 163 | ATGGAGACCGACACACTGTTGCTGTGGGTACTCCTCCTGTGGGTGCCAGGAAGCACGGGCGA |
| | TAAAACCCACACATGCCCTCCATGCCCTGCTCCAGAGGCCGCCGGTGGGCCATCAGTTTTCT |
| | TGTTTCCGCCTAAACCAAAGGACACGCTTATGATCTCCAGGACCCCCGAAGTTACGTGTGTG |
| | GTGGTTGATGTTAGTCACGAGGACCCGGAAGTCAAGTTCAACTGGTACGTTGATGGTGTAGA |
| | GGTGCACAATGCAAAGACGAAGCCACGCGAAGAACAATACAACAGCACATATCGAGTTGTGA |
| | GCGTACTCACGGTACTGCATCAGGACTGGCTGAACGGTAAAGAATACAAATGTAAAGTCTCC |
| | AATAAGGCACTTCCTGCGCCGATAGAAAAAACGATCAGTAAGGCCAAGGGCCAACCCCGAGA |
| | ACCACAGGTATATACGCTCCCACCGTCACGAGACGAGTTGACAAAAAATCAGGTCTCCCTGA |
| | CTTGCCTCGTGAAAGGTTTTTATCCCTCAGATATTGCTGTTGAGTGGGAAAGCAATGGGCAG |
| | CCAGAGAATAATTATAAGACGACTCCTCCGGTTTTGGATTCCGACGGTAGTTTTTTCTTGTA |
| | TAGTAAGCTTACTGTAGACAAGTCAAGATGGCAACAAGGTAATGTGTTCTCTTGCTCAGTTA |
| | TGCATGAAGCTCTTCATAACCATTACACGCAAAAGAGTCTCAGTCTGAGCCCCGGTGGCGGT |
| | AGCGACAGTTGGCAGGAAGAGGTGATTAAGTTGTGCGGTCGCGAGCTCGTTCGGGCCCAAAT |
| | TGCAATCTGCGGAAAATCTACGGGCGGAGAGGGCGGGGGTGAGGAGGGTGGGGGTGAAGGTG |
| | GGCAGCTCTATAGCGCCCTTGCGAATAAATGTTGTCACGTCGGATGCACAAAGAGGTCCCTC |
| | GCCAGGTTCTGC |
| 164 | ATGGAAACTGACACACTGTTGCTGTGGGTGCTGCTCCTTTGGGTACCCGGATCAACCGGGGA |
| | TAAGACCCACACTTGCCCCCCTTGCCCTGCCCCCGAAGCGGCCGGAGGTCCTTCAGTATTTT |
| | TGTTTCCACCGAAACCCAAAGATACTTTGATGATATCAAGAACTCCTGAAGTCACCTGCGTG |
| | GTAGTTGACGTATCTCATGAGGATCCCGAGGTGAAGTTCAATTGGTACGTCGATGGCGTCGA |
| | GGTTCATAACGCTAAGACTAAGCCGAGGGAAGAGCAATATAATTCCACTTATAGGGTGGTGT |
| | CCGTCTTGACTGTTTTGCACCAGGATTGGTTGAACGGGAAAGAGTACAAATGTAAGGTGAGT |
| | AATAAAGCTTTGGCTGCTCCCATCGAAAAGACAATAAGCAAGGCCAAGGGGCAACCTCGGGA |
| | GCCGCAGGTGTACACCCTTCCTCCCAGTAGAGACGAACTGACAAAAAACCAGGTGTCCCTGA |
| | CCTGCCTTGTGAAGGGGTTTTACCCGAGCGACATAGCGGTTGAATGGGAGAGCAACGGGCAA |
| | CCCGAGAACAACTACAAAACTACACCGCCTGTCCTGGACTCCGATGGAAGCTTCTTCCTCTA |
| | CTCCAAACTGACCGTGGACAAAAGCAGATGGCAACAAGGAAACGTATTCTCATGCTCAGTAA |
| | TGCACGAAGCATTGCACAATCACTACACCCAAAAGTCCCTCTCACTCTCCCCTGGTAAGGGC |
| | GGATCAGACTCATGGCAAGAGGAGGTAATTAAGTTGTGCGGGAGGGAGCTCGTCCGCGCGCA |
| | AATAGCCATTTGTGGCAAGTCCACTGGAGGAGGCGAGGGTGGAGGAGAGGGTGGTGGGGAGG |
| | GCAGGCAACTCTACAGTGCGCTCGCCAATAAATGCTGCCATGTTGGGTGCACGAAGCGCAGT |
| | CTCGCACAATTCTGC |
| 165 | ATGGAGACCGACACTCTGCTGCTCTGGGTACTCTTGCTGTGGGTGCCTGGGTCTACTGGGGA |
| | TAAGACCCACACGTGTCCTCCATGTCCGGCACCGGAGGCTGCTGGCGGGCCTTCTGTATTCC |
| | TCTTCCCACCCAAGCCAAAAGACACATTGATGATATCAAGGACGCCGGAAGTCACCTGTGTT |
| | GTTGTGGACGTTTCCCATGAAGACCCAGAGGTAAAATTCAATTGGTATGTGGACGGCGTAGA |
| | GGTTCACAACGCCAAAACCAAACCCCGAGAGGAACAGTATAATAGCACATATCGAGTAGTAT |
| | CTGTTCTCACAGTGCTCCATCAAGACTGGCTTAATGGTAAAGAGTATAAATGCAAAGTTTCC |
| | AATAAAGCCCTCGCTGCACCGATCGAGAAGACAATCAGTAAAGCGAAGGGCCAGCCTCGGGA |
| | ACCGCAGGTGTATACTCTTCCACCCTCAAGAGACGAGCTCACTAAAAACCAAGTTTCATTGA |
| | CATGCCTCGTCAAAGGTTTCTACCCATCAGACATCGCGGTCGAATGGGAAAGTAATGGGCAG |
| | CCGGAAAACAACTATAAAACGACGCCGCCCGTCTTGGATTCTGATGGTTCATTTTTTCTTTA |
| | CTCTAAATTGACCGTCGATAAAAGTAGGTGGCAACAAGGAAATGTTTTTTCCTGCTCCGTCC |
| | TGCATGAAGCGTTGCACAGTCACTATACCCAGAAGAGTCTTTCTTTGTCACCCGGAAAAGGC |
| | GGTTCAGATTCATGGCAGGAAGAAGTAATTAAACTCTGTGGCCGCGAGCTTGTTAGGGCGCA |
| | GATAGCCATATGTGGTAAAAGCACCGGAGGAGGTGAAGGCGGAGGCGAAGGAGGTGGGGAAG |
| | GAAGACAATTGTATTCTGCACTTGCAAATAAATGCTGTCATGTGGGGTGCACGAAACGCAGT |
| | CTTGCACAATTTTGT |
| 166 | ATGGAAACCGATACGCTGCTTCTTTGGGTTCTTCTCCTCTGGGTTCCAGGGTCCACCGGCGA |
| | CAAAACCCATACCTGCCCCCCTTGCCCTGCACCAGAAGCGGCGGGAGGACCTAGCGTTTTTC |
| | TTTTTCCTCCGAAACCGAAAGATACCCTCATGATATCAAGAACACCTGAGGTTACTTGCGTT |
| | GTCGTGGACGTGAGTCACGAAGACCCCGAGGTGAAGTTCAACTGGTATGTAGATGGAGTGGA |
| | GGTCCATAATGCAAAAACGAAACCGAGAGAAGAACAATACAACTCTACATATCGAGTCGTGT |
| | CAGTACTCACGGTTTTGCATCAAGATTGGCTGAACGGTAAGGAGTACAAGTGTAAGGTTAGC |
| | AACAAGGCTCTCGCGGCGCCGATAGAAAAGACTATAAGTAAAGCAAAAGGCCAGCCCAGAGA |
| | ACCTCAAGTTTACACTCTGCCTCCCAGCAGAGATGAACTGACTAAAAATCAGGTTTCATTGA |
| | CCTGTCTCGTCAAGGGTTTTTATCCAAGCGACATAGCAGTTGAATGGGAAAGCAACGGTCAA |
| | CCAGAAAATAATTACAAAACCACTCCACCAGTCTTGGACTCTGACGGATCCTTCTTTCTCTA |
| | TTCAAAATTGACGGTGGATAAATCTAGGTGGCAGCAAGGCAACGTCTTCTCTTGTAGCGTTA |
| | TGCATGAGGCGCTGCACAACCACTACACACAAAAGTCTCTTAGTTTGAGCCCGGGCGGCGGA |
| | AGCGACTCTTGGCAAGAGGAAGTGATAAAACTCTGTGGTCGAGAATTGGTACGCGCGCAGAT |
| | CGCTATCTGCGGCAAGTCCACAGGGGGAGGGGAAGGTGGCGGGGAAGGTGGTGGCGAGGGCA |
| | GGCAGTTGTATAGTGCACTTGCCAACAAGTGCTGCCATGTGGGGTGCACCAAGCGCAGTTTG |
| | GCACGGTTCTGC |
| 167 | ATGGAAACGGACACCCTTCTGCTCTGGGTACTGCTGCTCTGGGTTCCTGGTTCTACCGGTGA |
| | TAAAACTCACACTTGTCCCCCGTGTCCGGCACCAGAAGCCGCAGGAGGGCCATCTGTCTTTC |
| | TTTTTCCCCCAAAACCCAAGGATACACTGATGATCTCCCGCACTCCCGAAGTTACTTGTGTC |
| | GTAGTAGACGTTTCTCACGAGGACCCAGAGGTGAAATTCAATTGGTATGTTGACGGAGTAGA |
| | GGTGCATAATGCCAAGACAAAGCCCCGAGAGGAACAATACAATTCAACCTACAGAGTAGTGT |
| | CCGTTCTTACGGTTCTCCATCAGGATTGGCTCAACGGTAAGGAATATAAGTGCAAGGTAAGC |
| | AACAAAGCGCTGGCCGCACCCATTGAGAAAACCATTTCAAAAGCTAAAGGCCAACCCCGCGA |
| | ACCACAAGTTTATACTCTCCCCCCAAGTCGCGATGAACTTACAAAAAATCAAGTCTCATTGA |
| | CGTGCTTGGTCAAAGGCTTCTACCCGAGCGATATCGCTGTTGAATGGGAGTCTAATGGACAA |
| | CCGGAAAATAACTATAAAACTACACCCCCAGTCCTCGATTCAGACGGCAGCTTCTTCCTGTA |
| | TTCAAAACTGACGGTTGACAAATCACGCTGGCAACAGGGTAACGTTTTTTCCTGTAGCGTTC |
| | TTCATGAAGCCTTGCACAGTCACTACACCCAGAAGTCCCTTAGCTTGTCACCTGGCGGGGGT |
| | TCAGACTCTTGGCAGGAGGAGGTAATCAAACTGTGCGGAAGAGAACTGGTGAGGGCTCAGAT |
| | TGCAATTTGTGGGAAGAGCACGGGTGGCGGTGAAGGAGGTGGCGAGGGCGGAGGAGAGGGGA |
| | GGCAACTCTACAGTGCGTTGGCTAATAAATGCTGTCACGTCGGCTGTACTAAGAGAAGCCTC |
| | GCCAGATTTTGC |
| 168 | ATGGAAACAGATACTTTGTTGCTGTGGGTACTCCTCCTCTGGGTACCTGGGAGCACCGGGGA |
| | CAAGACGCATACTTGCCCTCCGTGCCCTGCACCAGAAGCCGCTGGTGGCCCATCTGTGTTTT |
| | TGTTCCCCCCTAAGCCAAAAGACACATTGATGATTTCACGAACTCCAGAAGTGACTTGCGTA |
| | GTTGTTGACGTATCACACGAAGACCCCGAGGTTAAATTTAATTGGTATGTGGACGGGGTCGA |
| | GGTGCATAACGCCAAAACCAAACCCCGGGAGGAACAATATAACTCTACGTATCGGGTCGTAT |
| | CTGTGTTGACCGTCCTTCACCAAGATTGGTTGAACGGCAAGGAATATAAGTGTAAAGTGTCT |
| | AATAAAGCATTGGCTGCCCCGATAGAAAAGACGATCTCTAAAGCCAAGGGCCAACCCAGAGA |
| | GCCTCAAGTATATACTCTCCCACCGAGTCGAGATGAGCTCACTAAGAACCAGGTGTCACTCA |
| | CGTGTCTGGTTAAAGGATTTTACCCTAGTGATATAGCCGTCGAGTGGGAATCAAATGGGCAG |
| | CCGGAGAATAACTATAAGACCACGCCTCCAGTTCTCGATTCCGATGGTAGCTTTTTCCTTTA |
| | CTCTAAACTTACGGTCGACAAGTCCAGGTGGCAACAGGGCAATGTATTTTCTTGCTCCGTCA |
| | TGCACGAGGCTTTGCACAACCATTACACGCAAAAGTCACTGTCCCTGTCTCCTGGAGGCGGT |
| | TCTGACAGTTGGCAGGAGGAGGTAATCAAATTGTGTGGGCGGGAGTTGGTTAGGGCGCAAAT |
| | TGCTATTTGCGGCAAAAGTACTGGGGGCGGTGAAGGCGGAGGCGAGGGAGGAGGAGAAGGTC |
| | GACAACTGTATTCTGCCTTGGCGAACAAATGCTGTCACGTCGGCTGTACGAAACGGTCTTTG |
| | GCCCAGTTTTGT |
| 169 | ATGGAAACTGACACTCTTCTGTTGTGGGTCCTTCTGCTGTGGGTTCCTGGCTCTACTGGAGA |
| | TAAGACACACACTTGTCCGCCATGCCCTGCGCCGGAAGCGGCGGGAGGACCGTCCGTTTTCC |
| | TGTTCCCTCCCAAACCCAAAGACACGTTGATGATTAGTCGCACGCCAGAAGTTACGTGCGTT |
| | GTCGTAGATGTATCCCACGAAGACCCCGAGGTGAAGTTCAATTGGTATGTAGATGGGGTGGA |
| | GGTCCATAACGCTAAGACCAAACCACGCGAGGAACAATATAATTCTACGTACCGCGTAGTGA |
| | GCGTTCTCACAGTTCTTCACCAGGATTGGCTTAACGGCAAGGAGTATAAGTGTAAGGTGTCT |
| | AATAAGGCCTTGGCTGCCCCGATCGAAAAAACGATAAGTAAAGCAAAGGGTCAACCTAGAGA |
| | ACCCCAAGTGTACACTCTCCCGCCATCACGGGATGAATTGACTAAGAACCAAGTGTCACTCA |
| | CGTGTCTTGTAAAGGGCTTCTACCCATCCGATATAGCCGTTGAGTGGGAATCCAATGGTCAG |
| | CCAGAGAACAACTATAAGACAACTCCGCCCGTACTTGATAGTGACGGTTCCTTTTTCCTTTA |
| | CAGTAAATTGACGGTAGATAAGTCTCGCTGGCAGCAAGGAAACGTCTTTTCTTGTTCAGTGC |
| | TTCATGAGGCGCTTCACTCACACTATACTCAGAAGAGTTTGAGTTTGTCTCCAGGTGGAGGC |
| | AGCGACTCATGGCAAGAGGAAGTAATCAAACTGTGTGGTCGCGAATTGGTACGAGCACAGAT |
| | CGCGATCTGCGGGAAATCAACAGGTGGCGGCGAAGGCGGCGGGGAAGGCGGCGGCGAAGGTA |
| | GGCAACTTTACTCAGCCCTTGCGAACAAATGTTGCCACGTAGGCTGTACTAAGAGAAGTCTC |
| | GCCCAGTTTTGC |
| 170 | ATGGAGACAGATACCCTTCTGTTGTGGGTCCTTCTGCTTTGGGTGCCGGGAAGTACAGGCGA |
| | CAAGACTCATACCTGCCCCCCTTGTCCAGCACCAGAAGCAGCTGGCGGGCCAAGCGTGTTCC |
| | TGTTTCCACCTAAGCCCAAAGATACGTTGATGATCAGCCGCACCCCGGAAGTAACCTGTGTA |
| | GTAGTAGATGTGTCCCACGAAGACCCCGAAGTAAAGTTTAATTGGTACGTCGATGGTGTCGA |
| | AGTACATAACGCTAAAACGAAGCCCCGAGAAGAGCAGTACAACAGTACTTACAGAGTAGTTT |
| | CTGTTCTTACAGTGCTGCATCAGGATTGGCTGAACGGGAAGGAGTATAAATGTAAAGTCTCA |
| | AACAAGGCACTTGCGGCACCAATAGAGAAGACAATATCTAAGGCCAAAGGGCAGCCTAGAGA |
| | GCCACAAGTATATACGCTGCCCCCCAGCAGGGACGAGCTGACAAAGAACCAAGTGTCACTGA |
| | CCTGCCTTGTTAAGGGCTTCTATCCGAGTGATATTGCTGTTGAATGGGAAAGTAACGGACAG |
| | CCGGAGAACAACTATAAAACTACTCCACCCGTGTTGGATAGTGACGGTAGCTTTTTTCTGTA |
| | CTCCAAGTTGACGGTAGACAAAAGTCGGTGGCAGCAGGGGAACGTATTTTCTTGTTCTGTCA |
| | TGCACGAAGCTCTTCACAATCACTATACGCAGAAGTCCCTCTCTCTCTCTCCTGGGAAGGGT |
| | GGTTCCGACAGCTGGCAGGAGGAGGTCATTAAACTGTGTGGTAGAGAGCTGGTACGGGCTCA |
| | AATTGCAATTTGTGGTAAGAGTACTGGCGGTGGCGAGGAAGGGGGTGGGGAGGAGGGCGGAG |
| | GTAGGCAGCTCTACTCTGCTCTCGCCAACAAGTGTTGTCACGTCGGGTGTACTAAAAGATCA |
| | CTTGCCCGCTTTTGT |
| 171 | ATGGAAACCGATACCCTGCTCTTGTGGGTCCTCCTGCTTTGGGTCCCAGGTTCCACAGGCGA |
| | CAAAACACATACATGCCCGCCGTGTCCGGCGCCTGAAGCAGCAGGAGGCCCCAGTGTATTCC |
| | TTTTCCCTCCAAAGCCAAAAGATACGTTGATGATATCTAGGACACCTGAGGTTACCTGCGTC |
| | GTAGTGGACGTATCCCACGAAGACCCAGAAGTCAAGTTTAACTGGTATGTGGACGGAGTGGA |
| | GGTACACAATGCAAAGACAAAGCCGCGAGAGGAACAATATAATTCCACCTATAGAGTCGTGT |
| | CAGTCCTTACGGTCTTGCACCAGGACTGGCTCAATGGTAAGGAGTATAAGTGCAAAGTATCA |
| | AACAAAGCTCTCGCAGCGCCCATCGAAAAGACCATCAGCAAAGCTAAGGGCCAGCCAAGAGA |
| | GCCTCAAGTGTACACGTTGCCGCCTTCAAGGGACGAGCTCACTAAAAATCAGGTATCACTTA |
| | CGTGTCTTGTCAAAGGGTTTTATCCTTCCGACATCGCGGTTGAATGGGAGAGCAATGGACAG |
| | CCGGAGAATAATTATAAAACGACGCCGCCGGTCCTTGACAGCGATGGTTCATTTTTCCTTTA |
| | CTCAAAGCTGACGGTTGATAAGTCTAGGTGGCAGCAGGGGAACGTCTTTTCCTGTAGTGTAC |
| | TTCATGAGGCGCTCCATTCTCATTACACTCAGAAGTCACTGAGCCTTTCACCCGGCAAAGGT |
| | GGATCAGACTCCTGGCAAGAAGAGGTAATCAAACTCTGTGGGAGGGAACTCGTTCGAGCCCA |
| | GATTGCAATCTGTGGGAAAAGCACAGGCGGAGGGGAAGAAGGGGGTGGCGAAGAAGGTGGGG |
| | GCAGGCAGCTCTATTCAGCTCTTGCCAACAAATGCTGTCATGTAGGCTGCACAAAGCGATCA |
| | CTGGCGAGATTCTGT |
| 172 | ATGGAAACCGACACCCTGCTGCTCTGGGTTCTTCTCCTCTGGGTTCCCGGCTCAACCGGAGA |
| | TAAAACTCATACTTGCCCACCCTGCCCGGCTCCCGAGGCAGCAGGTGGACCCTCAGTATTTT |
| | TGTTCCCTCCGAAACCTAAAGATACACTTATGATTAGCCGGACCCCTGAGGTAACGTGTGTG |
| | GTGGTTGACGTAAGTCATGAAGATCCAGAAGTAAAGTTTAACTGGTACGTAGACGGTGTGGA |
| | GGTACATAATGCGAAGACAAAACCACGAGAGGAACAGTATAACTCTACCTACCGCGTAGTAA |
| | GCGTACTTACTGTGCTCCACCAAGACTGGCTTAACGGGAAAGAGTATAAGTGTAAAGTCAGT |
| | AATAAAGCACTGGCCGCCCCGATCGAAAAAACAATCAGCAAGGCCAAAGGACAACCAAGGGA |
| | GCCTCAGGTCTATACTCTTCCCCCGAGTAGGGATGAGCTTACCAAGAACCAGGTGTCTCTGA |
| | CATGCCTTGTCAAGGGATTTTACCCGAGTGACATAGCCGTAGAATGGGAGTCAAACGGCCAA |
| | CCTGAAAACAACTATAAGACCACGCCTCCCGTACTCGACTCAGATGGAAGCTTTTTCCTCTA |
| | TAGCAAGCTGACCGTCGACAAAAGTAGGTGGCAACAGGGAAACGTCTTTAGTTGTTCCGTCA |
| | TGCACGAAGCTTTGCATAACCATTACACCCAGAAGAGTCTTTCCCTTTCCCCTGGCAAGGGG |
| | GGCTCCGACTCCTGGCAAGAGGAAGTAATCAAACTGTGTGGGCGCGAGCTTGTCCGCGCGCA |
| | AATAGCCATTTGCGGAAAAAGTACTGGAGGAGGAGAGGAAGGCGGCGGCGAGGAAGGTGGGG |
| | GCAGGCAGCTGTACAGTGCCTTGGCTAACAAGTGCTGCCATGTCGGCTGTACGAAAAGGTCT |
| | CTTGCTCAATTCTGT |
| 173 | ATGGAAACTGATACTCTTCTCCTTTGGGTGCTCCTCCTCTGGGTTCCCGGGTCCACAGGCGA |
| | TAAGACACATACCTGTCCACCCTGCCCAGCACCTGAAGCTGCAGGCGGCCCCAGCGTATTCC |
| | TGTTTCCTCCGAAGCCGAAAGACACACTTATGATTTCCCGGACGCCTGAGGTAACTTGCGTC |
| | GTAGTAGATGTGTCTCACGAAGACCCCGAGGTGAAATTCAACTGGTACGTTGATGGTGTGGA |
| | AGTTCATAATGCGAAAACTAAACCACGAGAGGAGCAATATAACTCAACTTATAGAGTTGTGA |
| | GCGTCTTGACGGTACTGCACCAGGACTGGCTGAATGGCAAAGAGTACAAATGCAAAGTCTCA |
| | AATAAGGCGTTGGCGGCTCCCATAGAGAAAACTATCAGCAAAGCCAAGGGTCAACCTCGGGA |
| | GCCACAAGTGTATACTCTTCCGCCTAGTCGCGACGAGCTCACAAAGAATCAGGTGAGTCTTA |
| | CTTGTTTGGTTAAGGGTTTCTACCCCAGTGACATTGCGGTCGAGTGGGAAAGTAACGGACAG |
| | CCTGAAAACAACTATAAAACAACGCCTCCAGTACTCGATTCAGATGGTTCATTCTTTCTTTA |
| | TTCCAAACTCACAGTCGACAAGAGTAGATGGCAACAAGGGAACGTGTTTAGCTGTAGCGTAC |
| | TCCATGAGGCACTCCACTCTCACTATACCCAAAAGTCTCTCAGCTTGTCACCCGGAAAAGGC |
| | GGTTCTGACAGTTGGCAAGAGGAAGTGATTAAATTGTGTGGGCGGGAACTTGTGAGGGCTCA |
| | AATCGCGATTTGCGGCAAGTCCACTGGTGGCGGCGAGGAAGGAGGAGGTGAAGAAGGAGGAG |
| | GTAGGCAACTGTATTCAGCGTTGGCGAATAAATGCTGCCATGTTGGATGTACTAAACGGAGC |
| | CTTGCTCAGTTCTGC |
| 174 | ATGGAAACTGACACCTTGTTGCTTTGGGTATTGCTTCTGTGGGTTCCGGGTAGCACGGGTGA |
| | TAAAACGCATACTTGCCCTCCTTGCCCGGCACCTGAAGCTGCCGGAGGTCCTTCCGTGTTCC |
| | TGTTCCCACCTAAGCCAAAAGACACACTTATGATTTCTCGCACACCAGAAGTAACGTGCGTC |
| | GTAGTTGACGTCTCCCATGAAGACCCGGAGGTAAAATTTAATTGGTACGTCGACGGGGTAGA |
| | AGTTCATAACGCAAAGACTAAACCACGAGAAGAGCAATACAACTCTACATACAGAGTAGTAA |
| | GCGTTCTCACCGTTCTTCATCAAGATTGGCTCAACGGAAAGGAGTATAAGTGTAAGGTGTCC |
| | AATAAAGCGTTGGCCGCACCAATCGAAAAGACCATAAGCAAAGCCAAAGGCCAACCCCGCGA |
| | ACCGCAGGTGTACACACTTCCCCCGTCCAGGGATGAATTGACAAAAAACCAAGTTTCCCTCA |
| | CGTGTCTCGTCAAGGGATTCTACCCGAGTGATATCGCAGTTGAATGGGAAAGCAATGGTCAG |
| | CCCGAGAATAACTACAAGACTACTCCCCCTGTGTTGGACTCAGACGGCTCATTCTTCCTCTA |
| | CAGTAAGTTGACTGTGGACAAAAGTCGGTGGCAGCAAGGCAATGTCTTCAGTTGTAGTGTAA |
| | TGCATGAAGCACTCCACAATCATTACACCCAAAAATCCCTGAGCCTGTCCCCGGGCGGAGGT |
| | TCAGATTCATGGCAGGAGGAAGTTATAAAACTGTGCGGGCGCGAGTTGGTGAGGGCGCAGAT |
| | CGCAATCTGTGGAAAGAGTACGGGAGGTGGCGAAGAGGGTGGTGGAGAAGAGGGAGGAGGTC |
| | GACAACTGTATTCCGCGCTCGCGAACAAGTGTTGCCACGTTGGCTGCACCAAACGAAGCCTG |
| | GCTCGATTTTGC |
| 175 | ATGGAGACTGACACCCTTCTCCTCTGGGTCCTCTTGCTTTGGGTCCCTGGCTCTACTGGTGA |
| | CAAGACACACACTTGTCCACCTTGCCCGGCTCCCGAGGCGGCAGGAGGACCAAGCGTTTTTC |
| | TGTTCCCTCCCAAACCAAAGGATACGCTTATGATCTCTCGAACGCCGGAAGTTACTTGCGTA |
| | GTAGTTGATGTCTCCCATGAAGATCCCGAAGTGAAGTTCAACTGGTATGTAGATGGTGTGGA |
| | AGTTCATAACGCGAAAACCAAACCACGCGAAGAACAGTATAACAGTACTTATCGGGTTGTTT |
| | CAGTACTCACGGTGCTCCATCAAGACTGGCTTAATGGAAAGGAGTATAAATGTAAGGTAAGT |
| | AACAAGGCATTGGCGGCTCCCATCGAGAAGACAATCTCCAAAGCAAAAGGGCAACCACGGGA |
| | GCCTCAGGTGTATACGTTGCCGCCCAGCAGAGATGAACTTACTAAGAATCAGGTGAGTCTCA |
| | CTTGTCTCGTCAAGGGCTTCTATCCCAGCGATATAGCCGTAGAATGGGAGAGTAACGGTCAG |
| | CCGGAGAACAACTACAAAACAACCCCGCCTGTTTTGGACTCCGATGGGAGTTTTTTTCTCTA |
| | CAGCAAACTCACGGTAGACAAAAGCAGGTGGCAGCAGGGCAATGTTTTCAGTTGCTCTGTTC |
| | TCCACGAAGCCCTCCACTCCCACTATACTCAGAAGTCTCTGAGTCTCTCACCAGGGGGAGGT |
| | AGCGATAGCTGGCAGGAGGAAGTGATCAAGTTGTGCGGGCGCGAACTCGTGCGGGCACAAAT |
| | TGCTATATGCGGTAAAAGTACGGGAGGTGGAGAGGAGGGTGGAGGTGAAGAAGGCGGTGGTA |
| | GACAATTGTATAGTGCGCTCGCCAACAAGTGTTGTCATGTCGGGTGTACGAAACGGTCCTTG |
| | GCGCGGTTTTGC |
| 176 | ATGGAAACTGACACACTTCTTCTGTGGGTACTCTTGTTGTGGGTTCCGGGCTCAACGGGTGA |
| | CAAGACACATACTTGTCCACCATGTCCCGCCCCAGAAGCTGCGGGAGGACCATCAGTTTTTT |
| | TGTTCCCCCCGAAACCGAAGGATACCCTCATGATAAGTCGAACGCCCGAAGTCACTTGCGTG |
| | GTGGTTGATGTTAGCCACGAGGACCCAGAAGTGAAGTTCAACTGGTACGTGGACGGGGTCGA |
| | AGTTCATAATGCGAAAACAAAGCCTCGCGAGGAACAGTACAACTCTACATACAGGGTTGTGT |
| | CTGTTTTGACAGTCTTGCACCAAGATTGGCTCAACGGGAAGGAATATAAGTGTAAGGTAAGC |
| | AATAAAGCACTGGCGGCCCCGATCGAAAAAACGATATCCAAGGCCAAGGGCCAGCCCCGAGA |
| | GCCTCAGGTATATACTCTGCCGCCAAGCCGGGATGAACTGACTAAAAACCAGGTCTCTTTGA |
| | CTTGTCTTGTCAAGGGATTTTACCCAAGTGACATTGCGGTAGAGTGGGAAAGCAACGGTCAA |
| | CCAGAAAACAATTACAAGACGACACCGCCGGTACTCGACTCAGATGGATCCTTTTTCCTGTA |
| | TAGCAAGCTGACAGTGGACAAGTCCCGGTGGCAGCAAGGGAACGTATTTTCATGCAGCGTGA |
| | TGCATGAGGCTCTTCACAACCATTACACACAGAAAAGTCTGTCATTGAGCCCTGGCGGCGGG |
| | AGCGATTCTTGGCAAGAAGAAGTTATAAAACTTTGCGGTCGAGAGCTGGTTCGGGCACAAAT |
| | TGCTATCTGCGGAAAATCTACAGGAGGAGGCGAGGAGGGAGGGGGCGAAGAAGGCGGGGGGA |
| | GACAGTTGTACAGTGCGCTCGCTAACAAGTGTTGCCACGTCGGTTGCACAAAGAGATCCCTG |
| | GCTCAATTCTGT |
| 177 | ATGGAGACAGATACTCTCTTGCTGTGGGTGCTGCTCTTGTGGGTTCCTGGAAGTACCGGTGA |
| | TAAAACTCACACCTGTCCCCCGTGTCCCGCACCAGAAGCGGCCGGTGGTCCCTCCGTTTTTC |
| | TCTTCCCTCCTAAACCTAAGGACACACTTATGATTAGCAGAACTCCAGAAGTTACGTGCGTA |
| | GTCGTTGACGTTAGTCATGAAGATCCTGAGGTTAAGTTCAACTGGTACGTAGACGGAGTAGA |
| | GGTCCACAACGCCAAGACGAAACCCCGAGAAGAGCAGTATAATTCTACCTATCGAGTTGTTT |
| | CAGTATTGACGGTGCTTCACCAAGATTGGCTGAATGGCAAAGAGTATAAGTGCAAGGTAAGC |
| | AACAAAGCACTCGCGGCTCCTATCGAGAAAACTATTTCCAAAGCTAAGGGCCAGCCTCGCGA |
| | ACCACAAGTCTATACCCTGCCACCGAGTCGGGACGAACTCACCAAGAACCAAGTGTCTCTTA |
| | CTTGCCTCGTTAAAGGTTTTTATCCCAGCGACATAGCCGTCGAATGGGAGTCCAATGGCCAA |
| | CCTGAGAACAACTATAAAACTACCCCTCCTGTACTTGATAGCGACGGAAGTTTTTTCCTCTA |
| | TTCAAAACTCACAGTTGATAAGTCTCGATGGCAACAGGGCAACGTCTTCTCTTGCAGTGTGT |
| | TGCATGAAGCTCTGCACTCTCATTACACACAGAAGAGTTTGTCTCTCAGTCCAGGTGGCGGC |
| | TCAGATAGCTGGCAGGAAGAAGTAATCAAGTTGTGCGGCAGGGAACTGGTAAGGGCACAGAT |
| | AGCCATTTGTGGAAAATCTACGGGTGGCGGTGAGGAAGGCGGCGGAGAAGAAGGGGGAGGTC |
| | GGCAGCTGTATAGTGCACTCGCAAACAAGTGCTGCCATGTCGGGTGCACCAAGCGATCCCTT |
| | GCCCAGTTTTGC |
| 178 | ATGGAAACTGATACGCTCCTCCTTTGGGTTCTTCTCCTCTGGGTACCCGGAAGCACTGGTGA |
| | CAAAACGCACACCTGTCCACCGTGTCCTGCTCCAGAGGCGGCCGGGGGACCGTCCGTTTTCC |
| | TTTTTCCTCCCAAACCTAAGGATACCCTTATGATCTCTCGCACGCCCGAGGTTACCTGTGTT |
| | GTGGTTGACGTGTCCCATGAAGACCCGGAAGTAAAATTTAATTGGTACGTGGACGGGGTCGA |
| | GGTTCATAACGCAAAGACCAAGCCACGAGAGGAGCAATATAATTCCACCTATCGCGTAGTCT |
| | CCGTCCTCACCGTGCTTCACCAGGATTGGCTCAACGGGAAGGAATACAAATGTAAAGTCAGT |
| | AATAAGGCTTTGGCGGCCCCGATTGAGAAGACTATAAGTAAGGCTAAGGGACAGCCACGAGA |
| | ACCGCAAGTTTATACATTGCCCCCCTCTAGGGATGAGTTGACTAAGAATCAGGTGTCACTCA |
| | CTTGTCTGGTAAAAGGGTTCTACCCGTCCGACATCGCTGTGGAATGGGAAAGCAATGGGCAA |
| | CCTGAAAATAATTATAAGACAACCCCTCCGGTGCTTGATAGCGACGGATCATTCTTTCTCTA |
| | TTCCAAGCTTACTGTAGATAAGAGTCGATGGCAACAGGGGAACGTATTCAGTTGCTCTGTTC |
| | TCCATGAGGCCCTGCATAGTCACTACACCCAAAAAAGCCTTAGTTTGAGTCCCGGGAAAGGA |
| | GGCTCCGATTCTTGGCAAGAAGAGGTAATAAAGCTGTGTGGACGAGAACTTGTCCGAGCACA |
| | AATTGCGATTTGTGGCAAATCTACAGGAGGGGGAGAAGGAGGCGGCGAAGGGGGAGGCGAGG |
| | GCAGGCAGGATTATTCCGCTCTGGCGAACAAATGTTGCCATGTTGGATGCACGAAACGAAGC |
| | CTGGCTCAGTTTTGC |
| 179 | ATGGAAACCGACACCCTCCTCCTTTGGGTACTTCTTCTCTGGGTTCCGGGTAGTACAGGAGA |
| | TAAAACGCATACCTGCCCACCGTGTCCTGCACCTGAGGCCGCTGGAGGACCTTCCGTCTTCC |
| | TCTTTCCACCCAAGCCGAAAGACACACTCATGATTAGTAGAACTCCAGAGGTCACGTGTGTT |
| | GTGGTGGACGTCAGTCACGAGGACCCTGAGGTTAAGTTCAACTGGTACGTTGATGGCGTAGA |
| | AGTCCACAATGCAAAGACCAAACCGAGAGAGGAGCAATATAACAGTACATATAGGGTTGTTA |
| | GCGTACTTACTGTTTTGCATCAAGACTGGTTGAATGGGAAGGAATATAAATGTAAAGTCTCC |
| | AACAAGGCTCTGGCTGCACCAATAGAAAAAACTATTTCTAAGGCAAAGGGTCAGCCTAGAGA |
| | GCCTCAAGTCTATACCTTGCCACCGTCAAGAGACGAGCTCACTAAAAATCAGGTGAGCCTGA |
| | CCTGTCTTGTGAAGGGCTTTTACCCGTCAGATATTGCCGTGGAGTGGGAATCAAACGGTCAG |
| | CCGGAGAATAACTACAAGACGACCCCACCAGTACTCGATAGCGATGGGTCTTTCTTTCTGTA |
| | CTCCAAGCTCACCGTGGACAAATCACGCTGGCAACAGGGCAACGTCTTTAGTTGCAGCGTAC |
| | TGCACGAGGCACTGCACAGCCACTACACACAAAAGAGTCTTTCTCTGTCTCCCGGTGGTGGC |
| | TCCGATAGTTGGCAGGAAGAAGTCATAAAGCTTTGTGGAAGAGAGCTTGTACGAGCGCAGAT |
| | TGCAATCTGCGGGAAGAGCACTGGAGGAGGTGAGGGAGGGGGTGAGGGCGGGGGCGAAGGAC |
| | GCCAGGACTATTCAGCACTTGCAAACAAATGCTGCCATGTAGGGTGTACGAAGCGCTCACTG |
| | GCCCGGTTTTGC |
| 180 | ATGGAGACCGACACTTTGCTGCTTTGGGTGTTGCTGCTGTGGGTCCCCGGTAGTACGGGAGA |
| | TAAAACACATACCTGCCCCCCATGCCCAGCCCCCGAAGCTGCAGGGGGGCCCTCTGTTTTCC |
| | TTTTTCCACCCAAACCTAAAGATACTCTGATGATTAGTCGGACTCCGGAAGTGACTTGCGTC |
| | GTTGTCGACGTCTCTCATGAGGATCCAGAAGTTAAGTTTAACTGGTATGTCGACGGGGTTGA |
| | GGTTCATAATGCAAAAACTAAACCGAGAGAAGAACAGTACAACTCTACTTATAGGGTTGTCA |
| | GTGTACTGACCGTCCTGCACCAGGATTGGCTTAACGGTAAGGAGTATAAGTGTAAAGTGTCC |
| | AATAAAGCCCTTGCCGCACCCATCGAGAAAACCATCTCCAAGGCAAAAGGACAGCCAAGGGA |
| | ACCGCAGGTATATACACTTCCGCCAAGCCGAGACGAACTTACGAAGAACCAGGTGTCTCTCA |
| | CGTGTCTCGTAAAAGGGTTTTATCCCAGCGATATCGCAGTTGAGTGGGAGAGCAATGGGCAG |
| | CCAGAGAATAATTATAAGACAACCCCTCCCGTGCTGGATTCAGACGGGAGTTTTTTTCTTTA |
| | CTCTAAGCTGACCGTAGACAAAAGTCGATGGCAGCAAGGCAACGTCTTTTCCTGCTCCGTTC |
| | TCCATGAAGCACTGCATAGCCATTATACCCAGAAGTCACTGAGCCTCTCTCCAGGGGGCGGG |
| | TCCGATTCATGGCAGGAAGAGGTAATCAAACTCTGTGGACGCGAACTGGTTCGCGCGCAGAT |
| | AGCGATTTGCGGCAAAAGCACAGGCGGTGGGGAAGGCGGTGGCGAGGGCGGTGGTGAAGGTC |
| | GACAAGATTATTCTGCTCTCGCTAACAAGTGTTGTCATGTAGGATGTACTAAAAGGAGTCTT |
| | GCGCAGTTCTGT |
| 181 | ATGGAGACGGACACTCTTCTCCTGTGGGTTCTCCTCTTGTGGGTTCCAGGATCTACCGGCGA |
| | TAAGACGCACACATGCCCACCCTGTCCTGCGCCTGAAGCCGCGGGGGGACCCAGCGTTTTTC |
| | TCTTCCCGCCGAAACCGAAAGACACACTTATGATCAGCCGGACTCCCGAGGTTACCTGCGTG |
| | GTGGTAGATGTATCTCACGAGGATCCCGAGGTCAAATTCAACTGGTACGTTGATGGGGTTGA |
| | AGTTCATAATGCCAAAACGAAGCCAAGAGAAGAGCAGTATAACTCCACATATAGAGTTGTTT |
| | CCGTCTTGACTGTTCTTCACCAAGATTGGCTGAATGGGAAGGAGTACAAATGTAAAGTTAGC |
| | AACAAGGCACTCGCCGCTCCCATTGAAAAAACTATAAGCAAAGCTAAGGGCCAACCGCGCGA |
| | ACCACAGGTCTACACGTTGCCGCCCTCTAGGGACGAACTCACGAAGAATCAGGTTTCCCTTA |
| | CCTGCCTCGTTAAAGGATTCTACCCCTCTGACATAGCGGTTGAATGGGAGAGCAACGGTCAG |
| | CCTGAGAACAACTACAAAACGACGCCTCCGGTGTTGGATTCCGACGGTAGTTTTTTCCTCTA |
| | TAGTAAGCTGACAGTGGATAAATCTCGGTGGCAGCAAGGGAATGTATTCTCCTGTTCAGTCC |
| | TGCATGAAGCCCTCCACTCCCATTATACACAGAAATCTCTTTCTCTGAGTCCCGGTAAAGGT |
| | GGGAGTGACTCTTGGCAGGAAGAGGTAATTAAGTTGTGTGGAAGGGAGCTGGTAAGAGCACA |
| | GATTGCCATCTGTGGCAAATCCACGGGCGGCGAAGGTGAGGGGGGTGAGGGGGAAGGGGGGT |
| | CCAGACAACTGTATTCTGCTCTGGCGAATAAGTGTTGCCATGTAGGGTGCACTAAACGGTCC |
| | TTGGCGCAGTTCTGT |
| 182 | ATGGAGACTGACACACTGCTCCTCTGGGTCCTTTTGCTCTGGGTTCCGGGGTCCACCGGTGA |
| | TAAAACTCATACGTGCCCACCTTGCCCCGCACCGGAGGCTGCTGGAGGACCCTCTGTCTTCC |
| | TGTTCCCGCCGAAGCCTAAAGACACATTGATGATCAGTCGAACACCGGAAGTCACCTGTGTA |
| | GTGGTTGATGTGAGCCATGAGGACCCTGAAGTAAAATTTAACTGGTATGTTGATGGCGTAGA |
| | AGTACACAACGCGAAGACTAAACCAAGGGAAGAGCAATACAACTCTACCTATAGGGTCGTTA |
| | GCGTACTGACTGTGCTTCACCAAGACTGGCTTAACGGGAAGGAGTACAAGTGCAAAGTGAGC |
| | AATAAGGCCCTCGCCGCGCCTATCGAGAAAACCATTTCCAAAGCCAAGGGTCAACCAAGGGA |
| | GCCTCAGGTTTACACCCTGCCCCCTTCAAGGGATGAGTTGACAAAAAACCAGGTAAGTCTGA |
| | CGTGTCTCGTTAAGGGATTCTACCCGTCAGATATCGCGGTAGAGTGGGAGAGCAACGGTCAG |
| | CCAGAAAATAATTACAAAACAACACCTCCAGTTTTGGACTCTGATGGGAGTTTTTTTCTTTA |
| | TTCTAAGTTGACAGTGGATAAGTCACGCTGGCAACAGGGGAACGTATTTAGCTGCTCAGTAC |
| | TTCATGAAGCGTTGCATTCTCACTACACACAGAAGAGCCTCTCCTTGAGTCCCGGAGGTGGC |
| | TCTGATTCTTGGCAGGAGGAGGTAATAAAACTTTGTGGTAGAGAACTGGTTCGCGCTCAGAT |
| | AGCTATTTGTGGAAAATCCACTGGCGGTGAAGGTGAAGGTGGAGAAGGAGAGGGCGGAAGCC |
| | GGCAGTTGTACTCTGCCCTGGCTAATAAGTGCTGTCACGTGGGCTGCACTAAGCGGAGCTTG |
| | GCAAGATTTTGC |
| 183 | ATGGAAACCGACACGCTGCTGCTGTGGGTGCTGTTGTTGTGGGTTCCAGGCTCAACTGGCGA |
| | TAAAACTCATACCTGTCCACCTTGTCCTGCGCCTGAGGCAGCTGGAGGGCCTAGCGTGTTCC |
| | TGTTCCCCCCCAAACCCAAAGACACGCTCATGATTAGCCGAACCCCTGAAGTGACCTGCGTT |
| | GTTGTGGACGTAAGCCACGAAGACCCCGAAGTTAAGTTTAATTGGTACGTCGACGGTGTTGA |
| | GGTTCATAACGCGAAGACTAAGCCGAGAGAGGAGCAATATAACAGCACCTACCGCGTAGTCT |
| | CAGTTCTTACCGTGCTCCACCAGGACTGGCTTAACGGGAAGGAATACAAATGCAAAGTTTCC |
| | AACAAAGCCTTGGCAGCCCCAATAGAGAAGACAATATCTAAGGCGAAAGGCCAACCGCGGGA |
| | ACCGCAAGTTTATACCCTCCCACCGAGCAGGGATGAGCTGACAAAAAATCAGGTTTCCCTCA |
| | CTTGTCTGGTCAAGGGATTTTATCCTTCAGACATAGCCGTTGAATGGGAGAGTAATGGGCAG |
| | CCGGAGAATAATTACAAGACCACCCCCCCGGTGTTGGACAGCGACGGTTCCTTCTTTCTCTA |
| | TTCTAAACTTACCGTCGACAAATCACGGTGGCAACAAGGAAATGTATTCTCATGCAGTGTAT |
| | TGCACGAAGCTCTGCACTCTCATTACACCCAAAAATCCCTCTCTCTCAGCCCTGGCGGTGGA |
| | TCTGATTCTTGGCAGGAAGAGGTGATTAAACTGTGTGGGCGAGAGCTTGTCCGAGCTCAGAT |
| | CGCTATTTGTGGCAAGAGTACCGGAGGCGAGGGTGAGGGAGGCGAAGGCGAGGGCGGAAGCC |
| | GGCAACTCTATAGCGCACTCGCTAATAAATGTTGTCATGTCGGCTGCACGAAGCGCTCACTG |
| | GCGCAGTTCTGC |
| 184 | ATGGAGACGGACACACTGCTCTTGTGGGTACTGCTCCTTTGGGTGCCAGGAAGTACAGGAGA |
| | CAAAACGCATACCTGTCCTCCATGCCCCGCTCCCGAGGCTGCCGGCGGACCAAGCGTATTTC |
| | TCTTCCCCCCTAAACCTAAAGACACATTGATGATAAGTAGGACGCCTGAAGTAACGTGTGTT |
| | GTCGTTGATGTAAGCCATGAAGATCCTGAAGTAAAGTTTAATTGGTATGTTGATGGCGTAGA |
| | AGTACATAACGCTAAGACGAAGCCACGGGAAGAGCAGTATAACTCAACTTACCGCGTTGTAA |
| | GCGTGCTTACCGTCCTGCATCAGGATTGGCTGAATGGTAAGGAATATAAGTGCAAAGTAAGC |
| | AACAAAGCATTGGCCGCACCAATAGAGAAGACGATTAGTAAAGCAAAAGGCCAGCCCAGAGA |
| | GCCGCAGGTTTATACACTTCCACCAAGCAGAGATGAACTTACGAAGAACCAGGTGTCTCTGA |
| | CTTGTCTGGTCAAGGGTTTCTATCCTTCCGACATTGCAGTGGAGTGGGAAAGCAATGGGCAG |
| | CCCGAAAACAATTATAAGACGACACCTCCAGTGTTGGACTCAGACGGTTCCTTTTTCTTGTA |
| | TTCCAAACTTACAGTGGATAAGTCAAGGTGGCAGCAAGGCAACGTATTTTCTTGTAGTGTTT |
| | TGCACGAAGCCCTGCATTCCCACTATACTCAAAAGAGCCTCAGTCTGTCCCCAGGAAAGGGA |
| | GGGAGTGACAGTTGGCAAGAGGAGGTAATAAAATTGTGTGGCAGAGAGCTTGTGCGCGCTCA |
| | GATCGCAATATGCGGGAAATCTACTGGGGGTGAGGGTGAGGGCGGCGAGGGAGAGGGGGGCA |
| | GTCGCCAAGATTATTCCGCCCTTGCGAATAAGTGTTGTCACGTCGGATGTACTAAGAGATCA |
| | TTGGCTCAGTTTTGT |

In some embodiments, any of the nucleotide sequences shown in Table 8 further comprise additional nucleotide sequence on their 5′ and/or 3′ ends. In some embodiments, any of the nucleotide sequences shown in Table 8 further comprise the nucleotide sequence ACGGGACCGATCCAGCCTCCGGACTCTAGAGCCACC (SEQ ID NO: 185) on their 5′ ends and/or any of the nucleotide sequences shown in Table 8 further comprise the nucleotide sequence TGATAAACCGGTTAGTAATGAGTTTGATATCTCGAC (SEQ ID NO: 186) on their 3′ ends.

### Pharmaceutical Compositions

The present disclosure provides pharmaceutical compositions comprising the fusion proteins or component peptides described herein or the nucleic acid molecules, or the expression vectors that encode them. The pharmaceutical compositions described herein are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also, Powell et al., “Compendium of excipients for parenteral formulations” PDA (1998) J Pharm Sci Technol 52:238-311.

The dose of the fusion proteins or component peptides described herein or the nucleic acid molecules, or the expression vectors that encode them administered to a patient may vary depending upon the age and the size of the patient, target disease, conditions, route of administration, and the like. The preferred dose is typically calculated according to body weight or body surface area. Depending on the severity of the condition, the frequency and the duration of the treatment can be adjusted. Effective dosages and schedules for administering the fusion proteins or component peptides described herein or the nucleic acid molecules, or the expression vectors that encode them may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition disclosed herein, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Any pharmaceutical composition described herein can be delivered subcutaneously or intravenously with a standard needle and syringe. In addition, with respect to subcutaneous delivery, a pen delivery device readily has applications in delivering a pharmaceutical composition disclosed herein. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see, Langer, supra; Sefton, 1987, CRC Crit. Ref Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending, or emulsifying any of the fusion proteins described herein in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients.

Such dosage forms in a unit dose include, for example, tablets, pills, capsules, injections (ampoules), suppositories, etc. The amount of the aforesaid fusion protein contained is generally about 5 to about 500 mg per dosage form in a unit dose; especially in the form of injection, it is preferred that the aforesaid fusion protein is contained in about 5 to about 100 mg and in about 10 to about 250 mg for the other dosage forms.

### Therapeutic Uses

### Monotherapy

The present disclosure provides methods comprising administering to a subject in need thereof a therapeutic composition comprising the fusion proteins or component peptides described herein or the nucleic acid molecules, or the expression vectors that encode them. The therapeutic composition can comprise any of the fusion proteins or component peptides as disclosed herein and a pharmaceutically acceptable carrier or diluent. As used herein, the expression “a subject in need thereof” means a human or non-human animal that exhibits one or more symptoms or indicia of a relaxin-2 associated disorder or disease, or who otherwise would benefit an increase or decrease in relaxin-2 activity. The fusion proteins or component peptides described herein and the expression vectors that encode them (and therapeutic compositions comprising the same) are useful, inter alia, for treating any disease or disorder in which activation or deactivation of RXFP1 is beneficial.

In certain embodiments, the present disclosure provides methods for activating RXFP1 on a cell surface, comprising administering an effective amount of the fusion proteins or component peptides described herein or the nucleic acid molecules, or the expression vectors that encode them to a subject in need thereof, thereby activating RXFP1 on the surface of the cell. Activation of RXFP1 on the cell surface can lead to cellular responses, including but not limited to, the elevation of cAMP levels, vasodilation, the expression of angiogenic factors, including VEGF, the expression of MMPs, and collagen degradation. In some embodiments, the cell is selected from the group consisting of endothelial cells, vascular smooth muscle cells, other vascular cells, cardiomyocytes, other cardiac cells, and fibroblasts.

This disclosure also provides methods for treating various relaxin-2 associated diseases. As used herein, the term “relaxin-2-associated disease,” is a disease or disorder that is caused by, or associated with, relaxin-2 protein production or relaxin-2 protein activity. The term “relaxin-2-associated disease” includes a disease, disorder or condition that would benefit from an increase in relaxin-2 protein activity. Non-limiting examples of relaxin-2-associated diseases include, for example, kidney diseases, including but not limited to, focal segmental glomerular sclerosis (FSGS), diabetic nephropathy, hepatorenal syndrome; fibrotic diseases, including but not limited to, scleroderma, idiopathic pulmonary fibrosis, renal fibrosis, cardiac fibrosis, NASH; cardiovascular diseases, including dilated cardiomyopathy, diastolic heart failure, pulmonary arterial hypertension, chronic heart failure, acute heart failure, congestive heart failure, coronary artery disease, hypertension, pre-eclampsia. Further details regarding signs and symptoms of the various diseases or conditions are provided herein and are well known in the art.

Administration of the compositions according to the methods described herein may result in a reduction of the severity, signs, symptoms, or markers of a relaxin-2-associated disease or disorder in a patient with a relaxin-2-associated disease or disorder. By “reduction” in this context is meant a statistically significant decrease in such level. The reduction (absolute reduction or reduction of the difference between the elevated level in the subject and a normal level) can be, for example, at least about 20%, 25%, 30%, 35%, 40%, 45%, 50%, 55%, 60%, 65%, 70%, 75%, 80%, 85%, 90%, or 95%, or to below the level of detection of the assay used.

### Combination Therapies and Formulations

The present disclosure also provides compositions and therapeutic formulations comprising the fusion proteins or component peptides described herein or the nucleic acid molecules, or the expression vectors that encode them in combination with one or more additional therapeutically active components, and methods of treatment comprising administering such combinations to subjects in need thereof.

Exemplary additional therapeutic agents include any therapeutic agents that may be used for the treatment of any relaxin-2-related disorders described herein. Exemplary additional therapeutic agents that may be combined with or administered in combination with the fusion proteins or component peptides described herein or the nucleic acid molecules, or the expression vectors that encode them include, but are not limited to, angiotensin II receptor blockers, e.g., azilsartan, candesartan, eprosartan, losartan, ACE inhibitors, e.g., lisinopril, benazepril, captopril, enalapril, moexipril, perindopril, quinapril, trandolapril, calcium channel blockers, e.g., amlodipine, amlodipine and benazepril, amlodipine and valsartan, diltiazem, felodipine, isradipine, nicardipine, nimodipine, nisoldipine, verapamil, or diuretics, e.g., chlorthalidone, hydrochlorothiazide, metolazone, indapamide, torsemide, furosemide, bumetanide, amiloride, triamterene, spironolactone, eplerenone, aldosterone antagonist, e.g., spironolactone, eplerenone, digoxin, e.g., lanoxin, beta blockers, e.g., carvedilol, metoprolol, bisoprolol.

In some embodiments, the additional therapeutic agents are drugs effective in treating fibrosis, including but not limited to, small molecule drugs and antibodies. Exemplary anti-fibrosis drugs include, but are not limited to, TGF-β inhibitors, e.g., small molecules such as hydronidone, distiertide, or antibodies such as fresolimumab, PDGF or VEGF antagonist, e.g., small molecules such as imatinib, nilotinib, or any drugs that target extracellular factors that are involved in the pathogenesis of fibrosis. The description of exemplary drugs for fibrosis can be found, e.g., Li et al., “Drugs and Targets in Fibrosis, Frontiers in Pharm.” 8: Article 855 (2007), incorporated herein by reference.

The additional therapeutically active component(s) may be administered just prior to, concurrent with, or shortly after the administration of the fusion proteins or component peptides described herein or the nucleic acid molecules, or the expression vectors that encode them.

The present disclosure provides pharmaceutical compositions in which the fusion proteins or component peptides described herein or the nucleic acid molecules, or the expression vectors that encode them is co-formulated with one or more of the additional therapeutically active component(s) as described elsewhere herein.

### Administration Regimens

In some embodiments, multiple doses of the fusion proteins or component peptides described herein or the nucleic acid molecules, or the expression vectors that encode them may be administered to a subject over a defined time course. The methods according to this aspect of the disclosure comprise sequentially administering to a subject multiple doses of the fusion proteins or component peptides described herein or the nucleic acid molecules, or the expression vectors that encode them. As used herein, “sequentially administering” means that each dose of the fusion proteins or component peptides described herein or the nucleic acid molecules, or the expression vectors that encode them is administered to the subject at a different point in time, e.g., on different days separated by a predetermined interval (e.g., hours, days, weeks, or months). The present disclosure provides methods which comprise sequentially administering to the patient a single initial dose of a fusion proteins or component peptides described herein or the nucleic acid molecules, or the expression vectors that encode them, followed by one or more secondary doses of the fusion proteins or component peptides described herein or the nucleic acid molecules, or the expression vectors that encode them, and optionally followed by one or more tertiary doses of the fusion proteins or component peptides described herein or the nucleic acid molecules, or the expression vectors that encode them.

The terms “initial dose,” “secondary doses,” and “tertiary doses,” refer to the temporal sequence of administration of the fusion proteins or component peptides described herein or the nucleic acid molecules, or the expression vectors that encode them. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the fusion proteins or component peptides described herein or the nucleic acid molecules, or the expression vectors that encode them, but generally may differ from one another in terms of frequency of administration. In certain embodiments, however, the amounts of fusion proteins or component peptides described herein or the nucleic acid molecules, or the expression vectors that encode them contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of fusion proteins or component peptides described herein or the nucleic acid molecules, or the expression vectors that encode them, which is administered to a patient prior to the administration of the very next dose in the sequence with no intervening doses.

The methods according to this aspect of the disclosure may comprise administering to a patient any number of secondary and/or tertiary doses of fusion proteins or component peptides described herein or the nucleic acid molecules, or the expression vectors that encode them. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose.

Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

In one embodiment, one or more of the fusion proteins or component peptides described herein or the nucleic acid molecules, or the expression vectors that encode them are administered to a subject as a weight-based dose. A “weight-based dose” (e.g., a dose in mg/kg) is a dose of the protein or peptides that will change depending on the subject's weight.

In another embodiment, one or more of the fusion proteins or component peptides described herein or the nucleic acid molecules, or the expression vectors that encode them, is administered to a subject as a fixed dose. A “fixed dose” (e.g., a dose in mg) means that one dose of the fusion proteins or component peptides described herein or the nucleic acid molecules, or the expression vectors that encode them is used for all subjects regardless of any specific subject-related factors, such as weight. In one particular embodiment, a fixed dose of fusion proteins or component peptides described herein or the nucleic acid molecules, or the expression vectors that encode them is based on a predetermined weight or age.

In general, a suitable dose of the fusion proteins or component peptides described herein or the nucleic acid molecules, or the expression vectors that encode them can be in the range of about 0.001 to about 200.0 milligram per kilogram body weight of the recipient, generally in the range of about 1 to 50 mg per kilogram body weight. For example, the fusion proteins or component peptides described herein or the nucleic acid molecules, or the expression vectors that encode them can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, about 50 mg/kg per single dose. Values and ranges intermediate to the recited values are also intended to be part of this disclosure.

In some embodiments, one or more of the fusion proteins or component peptides described herein or the nucleic acid molecules, or the expression vectors that encode them is administered as a fixed dose of between about 10 mg to about 2500 mg. In some embodiments, the fusion proteins or component peptides described herein or the nucleic acid molecules, or the expression vectors that encode them are administered as a fixed dose of about 10 mg, about 15 mg, about 20 mg, 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

### Kits

Any of the compositions described herein may be comprised in a kit. In a non-limiting example, the kit comprises one or more of the fusion proteins or component peptides described herein or the nucleic acid molecules, or the expression vectors that encode them.

The kit may further include reagents or instructions for using the fusion proteins or component peptides described herein or the nucleic acid molecules, or the expression vectors that encode them in a subject. It may also include one or more buffers.

The components of the kits may be packaged either in aqueous media or in lyophilized form. The container means of the kits will generally include at least one vial, test tube, flask, bottle, syringe, or other container means, into which a component may be placed, and preferably, suitably aliquoted. Where there is more than one component in the kit (labeling reagent and label may be packaged together), the kit also will generally contain a second, third, or other additional container into which the additional components may be separately placed. The kits may also comprise a second container means for containing a sterile, pharmaceutically acceptable buffer and/or other diluent. However, various combinations of components may be comprised in a vial. The kits of the present disclosure also typically include a means for containing the fusion proteins or component peptides described herein or the nucleic acid molecules, or the expression vectors that encode them, and any other reagent containers in close confinement for commercial sale.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

The examples of the present disclosure are offered by way of illustration and explanation, and are not intended to limit the scope of the present disclosure.

### Example 1. Heparin Chromatography for Relaxin-2 Fusion Protein Analogs

### Introduction

Heparin chromatography is a method commonly employed at early candidate screening to better understand a molecule's propensity to interact with elements of the vasculature when dosed in patients. Heparin and heparin sulfate proteoglycans are negatively charged polysaccharides present in vasculature and in tissues, of which positively charged molecules may bind at physiological pH (i.e., pI>7.4). Here, heparin chromatography was employed to screen for candidates/variants with reduced heparin binding, which is predictive of good PK properties. Materials used for the heparin chromatography are provided in Table 9.

TABLE 9
Materials

| Item | Vendor | Cat No. |
|---|---|---|
| POROS ™ Heparin | Thermo Fisher | 4333411 |
| 2.1 × 30 mm | | |
| Column | | |

### Methods

Mobile Phase A (Binding): 20 mM Tris pH 7.4 Mobile Phase B (Elution): 20 mM Tris pH 7.4+1 M NaCl Injection: 10 μg

### Detection: 220 nm

1. Equilibrated heparin column using mobile phase A for 10 minutes at 0.5 mL/min prior to analysis.

2. Diluted samples for analysis to 1 mg/mL with 20 mM Tris pH 7.4 to minimize ionic strength.

3. Ran the Heparin Chromatography method on the Agilent HPLC, using gradient shown in Table 10, below:

TABLE 10
HPLC Gradient

| Time | Flow | | |
|---|---|---|---|
| (min) | (mL/min) | % A | % B |
| 0 | 0.5 | 100 | 0 |
| 1 | 0.5 | 100 | 0 |
| 6 | 0.5 | 50 | 50 |
| 6.5 | 0.5 | 50 | 50 |
| 7 | 0.5 | 0 | 100 |
| 8 | 0.5 | 0 | 100 |
| 8.5 | 0.5 | 100 | 0 |
| 10 | 0.5 | 100 | 0 |

4. Included a positive control (no Heparin binding, Human IgG pool) and negative control (SE301 or AT1R).

5. Analyzed samples for retention time and reported relative retention time compared to the positive control (i.e., RT sample/RT positive control).

6. Calculated the approximate concentration of NaCl needed to elute using the following calculation:

$$[ NaCl ] = ( RT sample - 1 ) * 100$$

The results of the calculation are shown in Table 11.

TABLE 11
Retention Time, Relative Retention Time,
and NaCl Concentration for Samples

| Sample | RT | RRT | [NaCl] |
|---|---|---|---|
| Positive Control | 1.5 | N/A | 50 |
| Negative Control | 3.5 | 2.3 | 250 |
| Sample | 2.0 | 1.3 | 100 |

### Results

Table 12 shows the results of the heparin chromatography for a variety of relaxin-2 analog fusion proteins.

TABLE 12
Heparin Chromatography

| | | Heparin Chromatography | |
|---|---|---|---|
| | Theoretical | | ~[NaCl] (mM) |
| Sample | pI | RT | at elution |
| IgG | N/A | 2.0 | 20 |
| Prior fusion protein | 8.5 (9.4*) | 4.6 | 278 |
| SEQ ID NO: 53 | 8.2 | 3.9 | 208 |
| SEQ ID NO: 55 | 7.9 | 3.3 | 152 |
| SEQ ID NO: 56 | 7.9 | 3.4 | 163 |
| SEQ ID NO: 58 | 7.6 | 2.7 | 91 |
| SEQ ID NO: 59 | 7.6 | 2.8 | 99 |
| SEQ ID NO: 61 | 7.2 | 2.4 | 62 |
| SEQ ID NO: 62 | 7.2 | 2.5 | 70 |
| SEQ ID NO: 63 | 6.8 | 2.2 | 39 |
| SEQ ID NO: 64 | 6.8 | 2.2 | 43 |
| SEQ ID NO: 191 | 8.4 | 3.6 | 183 |
| SEQ ID NO: 192 | 8.3 | 3.2 | 140 |

*Experimentally determined using imaged capillary isoelectric focusing.

IgG is from Jackson ImmunoResearch (Catalog #009-000-003). The “Prior fusion protein” is a LALA IgG-RelB-Linker-RelA fusion with a theoretical pI of 8.5, but an experimentally determined pI of 9.4. Its linker protein comprises only one acidic amino acid. SEQ ID NOs: 53, 56, 58, 59, and 61-64 have linker proteins comprising at least two acidic amino acids as well as LALA IgG (SEQ ID NO: 50 or 201). The final two fusion proteins have linker proteins comprising only one acidic amino acid and have higher theoretical pI's. As shown in Table 12, above, there is a correlation between lower pI and lower non-specific binding found through heparin chromatography.

### Example 2. Low pI Relaxin-2 Fusion Protein Analogs Tend to Have Decreased Self Association as Measured by Affinity-Capture Self-Interaction Nanoparticle Spectroscopy (AC-SINS)

### INTRODUCTION

Understanding a molecule's propensity to self-associate is critical when evaluating biophysical properties of a development candidate. There are numerous ways to evaluate a molecule's propensity to self-associate, concentrating the molecule to high concentrations and evaluating by SEC (% Monomer) or measuring changes in turbidity (OD 340 nm), using DLS to calculate the second virial coefficient (B₂₂) or self-interaction coefficient (k_{d}), or using AC-SINS (Δλₘₐₓ). All three of these methods provide useful information but use different amounts of material to perform the evaluation. AC-SINS has emerged as a high throughput method for evaluating self-association using minimal material but still giving locally high concentrations by using affinity capture on gold nanoparticles. In short, gold nanoparticles are pre-coated with anti-human antibodies (Fc, Fab and H+L), which when incubated with target antibodies in dilute solutions, capture and concentrate in solution the antibody of interest. When the immobilized molecules of interest interact, the inter-particle distances decrease between gold nanoparticles, leading to increased plasmon wavelengths (i.e., red shift) that can be quantified using UV-VIS spectroscopy. Materials used for the spectroscopy are provided in Table 13.

TABLE 13
Materials

| Item | Vendor | Cat No. |
|---|---|---|
| 1M sodium acetate pH 4.3 | Molecular | MD2-019-PH |
| | Dimensions | |
| 1× DPBS | Gibco | 14190-136 |
| Panitumumab (Low Assoc Ctrl) | MyBioSource | MBS156169 |
| Ipilimumab (Med Assoc Ctrl) | MyBioSource | MBS156153 |
| Ganitumab (High Assoc Ctrl) | MyBioSource | MBS156142 |
| 20 nm gold nanoparticles | Ted Pella | 15705 |
| PEG methyl ether thiol (2 kDa) | Sigma | 729140 |
| Goat anti-Human IgG, Fcγ | Jackson | 109-005-098 |
| | ImmunoResearch | |
| Goat non-specific mAb | Jackson | 005-000-003 |
| | ImmunoResearch | |
| Zeba Desalting Columns 40K 5 mL | Thermo Fisher | 87770 |
| Zeba Desalting Columns 40K 2 mL | Thermo Fisher | 87768 |
| Zeba Desalting Columns 40K 0.5 mL | Thermo Fisher | 87766 |
| Costar 384-well Polystyrene plates | Fisher Scientific | 12-565-506 |
| 96-well Polypropylene plates | Grenier Bio-One | 652230P |

### Methods

### Preparing Buffer Solutions

To prepare 20 mM sodium acetate pH 4.3, 2 mL 1 M sodium acetate pH 4.3 stock was diluted to 100 mL with MilliQ water. pH was measured 4.3±0.1 and the solution was sterile filtered. The solution remained stable at room temperature for 1 month.

To 1 g PEG methyl ether thiol, was added 10 mL MilliQ water. This was vortexed briefly to suspend solids, making a 50 mM solution. To prepare a 10 μM solution for final dilution, the dilution scheme below was followed:
a. Dilute 50 mM stock to 1 mM (20 L 50 mM stock+980 L MilliQ water)
b. Dilute 1 mM step to 100 μM (10 L 1 mM stock+90 L MilliQ water)
c. Dilute 100 μM step to 10 μM (100 L 100 μM stock+900 L MilliQ water)
d. Volumes can be scaled according to number of samples to assay e. Remaining 50 mM stock should be aliquoted and kept at −20° C. until needed

### Preparing Gold NanoParticle Solution

Goat anti-human Fc IgG antibody (capture) and goat IgG antibody (non-capture) were buffer exchanged into 20 mM sodium acetate, pH 4.3. After buffer exchange, concentrations were normalized to 0.4 mg/mL for both antibodies.

A 4:1 volume ratio mixture of capture (anti-Fc):non-capture (Goat IgG) solution was prepared for 80% capture capacity coating solution to be used to incubate gold nanoparticles (AuNPs).

A 9:1 volume ratio of AuNPs:coating solution was made. The solution was incubated at room temperature, overnight in the dark.

After incubation, thiolated PEG was added to 0.1 μM final concentration from the diluted M stock to block empty sites on the AuNPs (i.e., 5 mL solution of AuNPs, add 50 L 10 M stock) and incubated at RT for one hour in the dark.

### Preparing AuNP Solution

2 mL of coated AuNP solution was centrifuged at 20,000×g for 15 minutes to sediment the AuNPs and 1800 L supernatant was carefully removed using a 1 mL pipette. The pelleted AuNPs were gently resuspended using a 200 L pipette to generate a 10× concentrated stock of coated AuNPs.

Preparing Target Antibody Solution (Either Method Follows this)

For each sample analyzed, 10 L of AuNP concentrate was incubated with 100 L antibody test solution (normalize to 0.05 mg/mL) at room temperature in the dark for 2 hours in a 96-well polypropylene plate. Two blank solutions were prepared with 10 L 10× AuNP concentrated to 100 L PBS for purposes of blanking the assay and determining wavelength shift upon addition of test antibody. Ganitumab was included as a positive control (high association, red shift) and Panitumumab as a negative control (no association, no UV shift). Each sample was prepared in duplicate for analysis.

After the 2-hour incubation, 100 L of resulting solution was transferred to a UV transparent polystyrene plate (384-well format). Two blank solutions were transferred to properly assess wavelength shift, then add duplicate standards and samples for analysis. The plate was then centrifuged for 1 minute at 1000×g to level the solutions in the wells.

Absorbance data are collected from 510 to 570 nm in 2 nm steps to determine wavelength shifts for each sample relative to AuNPs alone.

### Results

The results from ASCINS are shown below in Table 14.

TABLE 14
pI Variants Have Decreased Self-Association Propensity

| | Isoelectric | |
|---|---|---|
| | Point | |
| Sample | (Calculated) | Δλₘₐₓ |
| Prior fusion protein (Control) | 8.5 | 15.2 |
| Prior fusion protein (LALA) | 8.5 | 15.9 |
| SEQ ID NO: 53 | 8.2 | 7.5 |
| SEQ ID NO: 54 | 7.9 | 1.2 |
| SEQ ID NO: 55 | 7.9 | 7.0 |
| SEQ ID NO: 56 | 7.9 | 6.7 |
| Prior fusion protein (Control) | 8.5 | 15.2 |
| SEQ ID NO: 58 | 7.6 | 1.8 |
| SEQ ID NO: 59 | 7.6 | 1.9 |
| SEQ ID NO: 61 | 7.2 | −0.6 |
| SEQ ID NO: 62 | 7.2 | −0.4 |
| SEQ ID NO: 63 | 6.8 | 0.4 |
| SEQ ID NO: 64 | 6.8 | 0.4 |
| SEQ ID NO: 191 | 8.4 | 10.1 |
| SEQ ID NO: 192 | 8.3 | 6.1 |

As shown in Table 14, above, fusion proteins with low pI also have a tendency to show low self-aggregation.

### Example 3. Relaxin-2 Fusion Protein Analogs Induce cAMP Response in RXFP1 Transfected Cells

### Methods

HEK293 cells were seeded into a 96-well tissue culture plate followed by transient co-transfection with a human RXFP1 and a pGloSensor-22F plasmid. Transfected cells were stimulated by relaxin-2 or fusion protein analogs thereof, inducing Gs-mediated cAMP signaling. cAMP is assayed using the activity of the GloSensor biosensor, which is a mutant luciferase fused to a cAMP binding domain, leading to a production of light in the presence of its substrate luciferin. This readout of relative luminescent units (RLU) is used a proxy for cAMP response.

### Reagents

96-well tissue-culture treated plates. White with clear bottom. (Corning #3610)
HEK293 cells (ATCC CRL-1573)
Poly-D-lysine (Gibco A3890401)
DPBS (No calcium, no magnesium; Gibco 14190250)
DMEM (High glucose with L-glutamine and Sodium Pyruvate; Gibco 11995065)
TrypLE Express (Gibco 12605010)
FBS (HyClone™, Australian origin; Cytiva SH30084)
Penicillin-Streptomycin (Gibco 15140122)
C02-independent media (Gibco 18045088)
Opti-MEM™ I Reduced Serum media (Gibco 31985062)
pGloSensor™-22F cAMP plasmid (Promega Cat. #E2301)
D-luciferin, Potassium Salt (GoldBio LUCK-1G)
FuGENE HD transfection reagent (Promega #E2311)
Reservoirs (Corning/Axygen RES-V-25-SI)
Relaxin-2 (R&D Biosystems 6586-RN-025)
RXFP1-containing plasmid (pcDNA5/FRT/TO-human RXFP1, full-length)
Forskolin (Sigma F6886)
Plate reader capable of reading luminescence (CLARIOstar Plus)

### Reagent Preparation

### D-Luciferin, Potassium Salt:

D-luciferin was reconstituted in 10 mM HEPES, pH 7.5 at 25 mg/mL (78.5 mM; MW=318.4). This was aliquoted into single-use aliquots of ~200-500 μL in sterile microfuge tubes and stored at −80° C.

### Relaxin-2 Peptide:

Relaxin-2 peptides or relaxin-2 fusion protein analogs were reconstituted at 0.1 mg/mL in sterile DPBS (MW=5,986 Da, ε=12,865 M⁻¹ cm⁻¹) and measured at A₂₈₀ to determine final concentration. Aliquots were stored at −20° C.

### Forskolin:

Forskolin was reconstituted in 100% DMSO at 5 mM (2.05 mg/mL, MW=410.5). Aliquots were stored at −20° C.

cAMP Assay Media:

CO₂-independent media was pre-warmed to 37° C. using the bead bath. A single aliquot of D-luciferin was thawed and added at 5% final concentration (e.g., 4.75 mL cAMP assay media+250 μL of D-luciferin stock; gives 1.25 mg/mL or 3.93 mM final D-luciferin). This was used within the same day or discarded.

### Cell Culture and Maintenance

HEK293 cells (ATCC CRL-1573) were cultured in DMEM+10% FBS, 1% (iX or 10 U/mL) Pen-Strep in a humidified CO₂ incubator at 37 C, 5% CO₂ until 80-100% confluency. Cells were typically split 1:6 for 3 days and maintained in a sterile T-75 tissue culture flask.

cAMP Signaling Assay Protocol

This protocol is adapted from the GloSensor cAMP assay by Promega.

Raw data was exported to Excel using the MARS data analysis software that is opened following a run on the CLARIOstar plate reader. These values are measured in RLU, or relative luminescence units.

As shown in Table 15, all of the low prelaxin-2 fusion protein analogs were able to induce a cAMP response in RXFP1 transfected cells.

TABLE 15
cAMP Response in RXFP1 Transfected HEK293 Cells

| Agonist | pEC₅₀ | EC₅₀ in nM |
|---|---|---|
| Relaxin | 10.38 | 0.042 |
| Prior fusion protein (Control) | 9.51 | 0.31 |
| Prior fusion protein (LALA) | 9.49 | 0.33 |
| SEQ ID NO: 53 | 8.66 | 2.17 |
| SEQ ID NO: 54 | 8.10 | 7.87 |
| SEQ ID NO: 55 | 8.62 | 2.41 |
| SEQ ID NO: 56 | 8.72 | 1.90 |
| SEQ ID NO: 58 | 7.83 | 14.7 |
| SEQ ID NO: 59 | 7.75 | 17.7 |
| SEQ ID NO: 61 | 7.25 | 56.7 |
| SEQ ID NO: 62 | 7.18 | 65.8 |
| SEQ ID NO: 63 | 6.76 | 173.3 |
| SEQ ID NO: 64 | 7.02 | 96.2 |
| SEQ ID NO: 191 | 9.37 | 0.42 |
| SEQ ID NO: 192 | 8.84 | 1.46 |

### Example 4. In Vitro Characteristics of Relaxin-2 Fusion Protein Analogs

### Methods

### Heparin Chromatography:

Heparin chromatography was performed to understand the propensity of a relaxin-2 fusion protein analog to interact with elements of the vasculature and/or rapidly distribute into tissues when dosed in patients. Analogs that were found to bind heparin weakly may be predictive of good pharmacokinetic properties. Briefly, a heparin column was equilibrated using mobile phase A (20 mM Tris pH 7.4) for 10 minutes at 0.5 mL/min prior to analysis. 10 g per sample was run using the Heparin Chromatography method on an Agilent HPLC using 280 nm detection, using gradient shown in Table 16, below (mobile phase B: 20 mM Tris pH 7.4, 1 M NaCl):

TABLE 16
HPLC Gradient

| Time | Flow | | |
|---|---|---|---|
| (min) | (mL/min) | % A | % B |
| 0 | 0.5 | 100 | 0 |
| 6 | 0.5 | 50 | 50 |
| 7 | 0.5 | 0 | 100 |
| 8 | 0.5 | 0 | 100 |
| 8.5 | 0.5 | 100 | 0 |
| 10 | 0.5 | 100 | 0 |

A positive control (no Heparin binding, pembrolizumab) and negative control (mild Heparin binding, adalimumab) was included, and samples were analyzed for retention time and relative retention time compared to the positive control (i.e., RT sample/RT positive control). The approximate concentration of NaCl needed to elute was calculated using the following calculation:

$$[ NaCl ] = ( RT sample ) * 100$$

The results of the calculation are shown in Table 17:

TABLE 17
Retention Time and NaCl Concentration for Samples

| Sample | RT | [NaCl] |
|---|---|---|
| Positive Control | 1.5 | 150 |
| Negative Control | 3.5 | 350 |
| Sample | 2.0 | 200 |

Capillary Isoelectric Focusing (cIEF)

Imaged capillary isoelectric focusing (cIEF) was used to separate differentially charged molecules (i.e., relaxin-2 fusion protein analogs) using electrophoretic mobility in an ampholyte solution to determine their isoelectric points (pI). Molecules were loaded to a capillary and separated based on their pI by allowing molecules to migrate along an electrical field until the molecules reached the pH corresponding to their pI. UV absorption of the whole capillary was measured throughout the separation, which allowed for real-time observation as well as final quantification.

### Baculovirus Particle (BVP) ELISA

BVP ELISA was employed to understand the propensity of a relaxin-2 fusion protein analog for non-specific or non-target interactions. BVPs are empty viral capsids with no viral genome, but in the process of production, budding off from the cell membrane allows them to take components of the cell membrane along with them. Thus, the BVPs possess a highly diverse cell surface with many moieties present, which mimic what the molecule of interest (i.e., relaxin-2 fusion protein analog) may encounter in vivo. Briefly, BVPs are coated on a plate by adding 25 L of BVP solution to each well. BVP solution was made by diluting BVP stock (Medna Scientific; Cat. No. E3001) to 1×10⁶ PFU/mL with 0.1 M carbonate buffer, pH 9.6. Following overnight incubation at 5° C., BVP solution was blotted from wells and wells were washed three times with PBST. Plates were blocked with 100 μL/well of 1× BSA in PBS blocking buffer (Cepham Life Sciences; Cat. No. 10615). Plates were incubated at 25° C. on a plate shaker for 1 hour. Blocking solution was blotted from wells and wells were washed three times with PBST. Samples (i.e., relaxin-2 fusion protein analogs) were prepared in duplicate to cover dilution range from 3 μM to 0.1 nM and added to plates. Plates were incubated at 25° C. for 1 hour, after which the wells were blotted and washed three times with PBST. 25 L/well of 1:10,000 diluted detection monoclonal antibody (Peroxidase AffiniPure Goat Anti-Human IgG, Fcγ fragment specific; Jackson ImmunoResearch; Cat. No. 50-194-1564) was added, and plates were incubated at 25° C. for 1 hour, after which the wells were blotted and washed three times with PBST. 1-Step™ Ultra TMB-ELISA Substrate Solution (Life Technologies; Cat. No. 34029) was then added. After about 2 minutes, 25 L 2N HCl was added to quench the reaction, and a plate reader was used to analyze the plate at 450 nm with correction at 570 nm.

### Potency Assay

A transient hRXFP1 assay was performed substantially as described in Example 3.

### Affinity-Capture Self-Interaction Nanoparticle Spectroscopy (AC-SINS)

AC-SINS was performed to understand the propensity of a molecule (i.e., relaxin-2 fusion protein analog) to self-associate. The methodology performed was similar to the method described in Example 2. AuNP solution was prepared as follows: 1.5 mL of coated AuNP solution was centrifuged at 20,000×g for 5 minutes to sediment the AuNPs and 1,350 L was carefully removed using a 1 mL pipette. The pelleted AuNPs were gently resuspended using a 200 L pipette to generate a 10× concentrated stock of coated AuNPs. For each sample analyzed, 5 L of AuNP concentrate was incubated with 45 L antibody test solution (normalized to 0.05 mg/mL) at room temperature in the dark for 2 hours in a 384-well polypropylene plate. After the 2-hour incubation, absorbance data was collected from 450 nm to 650 nm in 1 nm steps to determine wavelength shifts for each sample relative to AuNPs alone.

### Nanoscale Differential Scanning Fluorimetry (NanoDSF)

NanoDSF was performed using the NanoTemper Prometheus Panta to investigate the conformational stability of a relaxin-2 protein fusion analog. Conformational stability was measured by applying a thermal ramp to a solution containing the molecule of interest, measuring the intrinsic fluorescence, backscattering, and using dynamic light scattering (DLS) to provide various thermal stability parameters.

### Results

The results are shown below in Table 18:

TABLE 18
In vitro Characteristics of Relaxin-2 Fusion Protein Analogs

| | Heparin | | | | Potency | | | |
|---|---|---|---|---|---|---|---|---|
| | Binding | | cIEF | BVP ELISA | Assay | AC- | NanoDSF | |
| | | [NaCl] | Isoelectric | Normalized | EC50 | SINS | Tₒₙₛₑₜ | Tₘ1 |
| Sample | RT | (mM) | point (pI) | Score | (nM) | Δλmax | (° C.) | (° C.) |
| Relaxin-2 | ND | ND | 9.4 | N/A | 0.1 | N/A | N/A | N/A |
| Prior fusion | ND | ND | 9.0 | 28 | 0.3 | 7 | 61.4 | 68.9 |
| (SEQ ID NO: | | | | | | | | |
| 224) | | | | | | | | |
| SEQ ID NO: | 3.88 | 330 | 8.9 | 2 | 2.5 | 8 | 60.5 | 70.4 |
| 204 | | | | | | | | |
| SEQ ID NO: | NT | NT | ND | ND | 12.3 | 1 | 63.3 | 70.7 |
| 205 | | | | | | | | |
| SEQ ID NO: | 3.32 | 280 | 8.5 | 1 | 2.9 | 7 | 62.8 | 70.6 |
| 206 | | | | | | | | |
| SEQ ID NO: | 3.43 | 290 | 8.5 | 1 | 2.5 | 7 | 61.6 | 70.6 |
| 57 | | | | | | | | |
| SEQ ID NO: | 2.71 | 230 | 7.9 | 1 | 16.0 | 2 | 62.9 | 70.9 |
| 207 | | | | | | | | |
| SEQ ID NO: | 2.79 | 240 | 7.9 | 1 | 15.8 | 2 | 63.0 | 71.0 |
| 60 | | | | | | | | |
| SEQ ID NO: | 2.42 | 210 | 7.5 | 1 | 60.3 | 0 | 63.9 | 71.1 |
| 208 | | | | | | | | |
| SEQ ID NO: | 2.5 | 210 | 7.1 | 1 | 62.8 | 0 | 63.6 | 71.2 |
| 209 | | | | | | | | |
| SEQ ID NO: | 2.19 | 190 | 7.1 | 1 | 140.0 | 0 | 62.4 | 71.0 |
| 210 | | | | | | | | |
| SEQ ID NO: | 2.23 | 190 | ND | 1 | 101.2 | 0 | 62.8 | 70.8 |
| 211 | | | | | | | | |
| SEQ ID NO: | 3.63 | 310 | ND | 1 | 0.5 | 10 | 62.9 | 70.8 |
| 212 | | | | | | | | |
| SEQ ID NO: | 3.2 | 270 | ND | 1 | 1.8 | 6 | 63.0 | 71.2 |
| 213 | | | | | | | | |
| SEQ ID NO: | 2.7 | 230 | 8.0 | 1 | 4.2 | 5 | 63.0 | 69.9 |
| 66 | | | | | | | | |
| SEQ ID NO: | 3 | 250 | 8.0 | 1 | 3.3 | 11 | 61.9 | 69.3 |
| 68 | | | | | | | | |
| SEQ ID NO: | 2.5 | 210 | 7.5 | 1 | 8.0 | 7 | 61.9 | 69.7 |
| 70 | | | | | | | | |
| SEQ ID NO: | 3.1 | 260 | 8.0 | 1 | 5.8 | 4 | 62.6 | 69.6 |
| 72 | | | | | | | | |
| SEQ ID NO: | 2.6 | 220 | 7.4 | 1 | 10.2 | 3 | 62.3 | 69.9 |
| 74 | | | | | | | | |
| SEQ ID NO: | 3 | 250 | 7.4 | ND | ND | ND | ND | ND |
| 76 | | | | | | | | |
| SEQ ID NO: | 2.3 | 200 | 7.1 | 1 | 23.4 | 4 | 60.9 | 69.9 |
| 78 | | | | | | | | |
| SEQ ID NO: | 2.3 | 200 | 7.5 | 1 | 117.0 | 0 | 61.9 | 70.0 |
| 79 | | | | | | | | |
| SEQ ID NO: | 2.6 | 220 | 7.4 | 1 | 162.0 | 0 | 62.6 | 69.8 |
| 80 | | | | | | | | |
| SEQ ID NO: | 2 | 170 | 7.1 | 1 | 426.0 | 0 | 62.8 | 70.2 |
| 81 | | | | | | | | |
| SEQ ID NO: | 2.6 | 220 | 7.5 | 1 | 18.9 | 4 | 62.3 | 70.1 |
| 82 | | | | | | | | |
| SEQ ID NO: | 2.8 | 240 | 7.5 | ND | 9.3 | 7 | 60.9 | 69.2 |
| 83 | | | | | | | | |
| SEQ ID NO: | 2.3 | 200 | 7.1 | 1 | 41.9 | 5 | 61.4 | 69.9 |
| 84 | | | | | | | | |
| SEQ ID NO: | 2.1 | 180 | 7.2 | 1 | 54.4 | 0 | 62.8 | 70.2 |
| 85 | | | | | | | | |

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

1-65. (canceled)

66. A method of producing a fusion protein, comprising culturing a host cell comprising a polynucleotide comprising a nucleotide sequence encoding a fusion protein, under conditions such that the fusion protein is produced, wherein the fusion protein comprises, from N-terminus to C-terminus:
a first peptide;
a linker peptide; and
a second peptide,
wherein:
(a) the first peptide comprises an amino acid sequence that differs at 0, 1, 2, 3, 4, or 5 amino acids when compared to the amino acid sequence of SEQ ID NO: 7 and the second peptide comprises an amino acid sequence that differs at 0, 1, 2, 3, 4, or 5 amino acids when compared to the amino acid sequence of SEQ ID NO: 9; or
the first peptide comprises an amino acid sequence that differs at 0, 1, 2, 3, 4, or 5 amino acids when compared to the amino acid sequence of SEQ ID NO: 9 and the second peptide comprises an amino acid sequence that differs at 0, 1, 2, 3, 4, or 5 amino acids when compared to the amino acid sequence of SEQ ID NO: 7; and
(b) the linker peptide comprises an amino acid sequence with 12-15 amino acids, comprising 2-5 acidic amino acids and 10-13 non-acidic amino acids.

67. The method of claim 66, wherein the linker peptide comprises an amino acid sequence selected from the group consisting of:
R₁R₁R₁R₂R₁R₁R₁R₂R₁R₁R₁R₂R₁;
R₁R₁R₁R₂R₁R₁R₁R₂R₁R₁R₁R₂R₁R₁R₁;
R₁R₁R₂R₁R₁R₁R₂R₂R₁R₁R₁R₂R₁R₁;
R₁R₁R₁R₂R₂R₁R₁R₁R₂R₂R₁R₁R₁; and
R₁R₁R₂R₁R₂R₁R₁R₂R₁R₂R₁R₁R₁,
wherein R₁ is a non-acidic amino acid and R₂ is an acidic amino acid.

68. The method of claim 66, wherein the linker peptide comprises an amino acid sequence selected from the group consisting of SEQ ID NOs: 14-23.

69. The method of claim 66, wherein each of the acidic amino acids in the linker peptide is aspartate or glutamate, each of the acidic amino acids in the linker peptide is aspartate, or each of the acidic amino acids in the linker peptide is glutamate; and/or the non-acidic amino acid(s) are glycine, proline, or serine.

70. The method of claim 66, wherein the first peptide comprises:
the amino acid sequence of DSWX₃EEVIKLCGRELVRAQIAICGX₄ST (SEQ ID NO: 3), wherein X₃ is methionine, lysine, or glutamine, and wherein X₄ is methionine or lysine;
the amino acid sequence of X₅QX₆YSALANKCCHVGCTKRSLAX₇FC (SEQ ID NO: 4), wherein X₅ is arginine or absent, X₆ is leucine or aspartate, and wherein X₇ is arginine, glutamine, or glutamate;
an amino acid sequence selected from the group consisting of SEQ ID NOs: 5-7; or
an amino acid sequence selected from the group consisting of SEQ ID NOs: 8-13.

71. The method of claim 66, wherein the second peptide comprises:
the amino acid sequence of DSWX₃EEVIKLCGRELVRAQIAICGX₄ST (SEQ ID NO: 3), wherein X₃ is methionine, lysine, or glutamine, and wherein X₄ is methionine or lysine;
the amino acid sequence of X₅QX₆YSALANKCCHVGCTKRSLAX₇FC (SEQ ID NO: 4), wherein X₅ is arginine or absent, X₆ is leucine or aspartate, and wherein X₇ is arginine, glutamine, or glutamate;
an amino acid sequence selected from the group consisting of SEQ ID NOs: 5-7; or
an amino acid sequence selected from the group consisting of SEQ ID NOs: 8-13.

72. The method of claim 66, wherein:
the first peptide comprises the amino acid sequence of SEQ ID NO: 5 and the second peptide comprises the amino acid sequence of SEQ ID NO: 8;
the first peptide comprises the amino acid sequence of SEQ ID NO: 5 and the second peptide comprises the amino acid sequence of SEQ ID NO: 9;
the first peptide comprises the amino acid sequence of SEQ ID NO: 5 and the second peptide comprises the amino acid sequence of SEQ ID NO: 10;
the first peptide comprises the amino acid sequence of SEQ ID NO: 5 and the second peptide comprises the amino acid sequence of SEQ ID NO: 11;
the first peptide comprises the amino acid sequence of SEQ ID NO: 5 and the second peptide comprises the amino acid sequence of SEQ ID NO: 12;
the first peptide comprises the amino acid sequence of SEQ ID NO: 5 and the second peptide comprises the amino acid sequence of SEQ ID NO: 13;
the first peptide comprises the amino acid sequence of SEQ ID NO: 6 and the second peptide comprises the amino acid sequence of SEQ ID NO: 8;
the first peptide comprises the amino acid sequence of SEQ ID NO: 6 and the second peptide comprises the amino acid sequence of SEQ ID NO: 9;
the first peptide comprises the amino acid sequence of SEQ ID NO: 6 and the second peptide comprises the amino acid sequence of SEQ ID NO: 10;
the first peptide comprises the amino acid sequence of SEQ ID NO: 6 and the second peptide comprises the amino acid sequence of SEQ ID NO: 11;
the first peptide comprises the amino acid sequence of SEQ ID NO: 6 and the second peptide comprises the amino acid sequence of SEQ ID NO: 12;
the first peptide comprises the amino acid sequence of SEQ ID NO: 6 and the second peptide comprises the amino acid sequence of SEQ ID NO: 13;
the first peptide comprises the amino acid sequence of SEQ ID NO: 7 and the second peptide comprises the amino acid sequence of SEQ ID NO: 8;
the first peptide comprises the amino acid sequence of SEQ ID NO: 7 and the second peptide comprises the amino acid sequence of SEQ ID NO: 9;
the first peptide comprises the amino acid sequence of SEQ ID NO: 7 and the second peptide comprises the amino acid sequence of SEQ ID NO: 10;
the first peptide comprises the amino acid sequence of SEQ ID NO: 7 and the second peptide comprises the amino acid sequence of SEQ ID NO: 11;
the first peptide comprises the amino acid sequence of SEQ ID NO: 7 and the second peptide comprises the amino acid sequence of SEQ ID NO: 12;
the first peptide comprises the amino acid sequence of SEQ ID NO: 7 and the second peptide comprises the amino acid sequence of SEQ ID NO: 13;
the second peptide comprises the amino acid sequence of SEQ ID NO: 5 and the first peptide comprises the amino acid sequence of SEQ ID NO: 8;
the second peptide comprises the amino acid sequence of SEQ ID NO: 5 and the first peptide comprises the amino acid sequence of SEQ ID NO: 9;
the second peptide comprises the amino acid sequence of SEQ ID NO: 5 and the first peptide comprises the amino acid sequence of SEQ ID NO: 10;
the second peptide comprises the amino acid sequence of SEQ ID NO: 5 and the first peptide comprises the amino acid sequence of SEQ ID NO: 11;
the second peptide comprises the amino acid sequence of SEQ ID NO: 5 and the first peptide comprises the amino acid sequence of SEQ ID NO: 12;
the second peptide comprises the amino acid sequence of SEQ ID NO: 5 and the first peptide comprises the amino acid sequence of SEQ ID NO: 13;
the second peptide comprises the amino acid sequence of SEQ ID NO: 6 and the first peptide comprises the amino acid sequence of SEQ ID NO: 8;
the second peptide comprises the amino acid sequence of SEQ ID NO: 6 and the first peptide comprises the amino acid sequence of SEQ ID NO: 9;
the second peptide comprises the amino acid sequence of SEQ ID NO: 6 and the first peptide comprises the amino acid sequence of SEQ ID NO: 10;
the second peptide comprises the amino acid sequence of SEQ ID NO: 6 and the first peptide comprises the amino acid sequence of SEQ ID NO: 11;
the second peptide comprises the amino acid sequence of SEQ ID NO: 6 and the first peptide comprises the amino acid sequence of SEQ ID NO: 12;
the second peptide comprises the amino acid sequence of SEQ ID NO: 6 and the first peptide comprises the amino acid sequence of SEQ ID NO: 13;
the second peptide comprises the amino acid sequence of SEQ ID NO: 7 and the first peptide comprises the amino acid sequence of SEQ ID NO: 8;
the second peptide comprises the amino acid sequence of SEQ ID NO: 7 and the first peptide comprises the amino acid sequence of SEQ ID NO: 9;
the second peptide comprises the amino acid sequence of SEQ ID NO: 7 and the first peptide comprises the amino acid sequence of SEQ ID NO: 10;
the second peptide comprises the amino acid sequence of SEQ ID NO: 7 and the first peptide comprises the amino acid sequence of SEQ ID NO: 11;
the second peptide comprises the amino acid sequence of SEQ ID NO: 7 and the first peptide comprises the amino acid sequence of SEQ ID NO: 12; or
the second peptide comprises the amino acid sequence of SEQ ID NO: 7 and the first peptide comprises the amino acid sequence of SEQ ID NO: 13.

73. The method of claim 66, wherein the fusion protein comprises an amino acid sequence selected from the group consisting of SEQ ID NOs: 25-48.

74. The method of claim 66, wherein the fusion protein further comprises an IgG Fc.

75. The method of claim 74, wherein:
the IgG Fc comprises the amino acid alanine at EU positions 234 and 235;
the IgG Fc comprises the amino acid alanine at EU position 329;
the IgG Fc comprises the amino acid alanine at EU positions 234, 235, and 329;
the IgG Fc comprises the amino acids alanine, alanine, alanine, leucine, and serine at EU positions 234, 235, 329, 428, and 434, respectively;
the IgG Fc comprises the amino acids lysine, phenylalanine, and tyrosine at EU positions 433, 434, and 436, respectively;
the IgG Fc comprises the amino acids tyrosine, threonine, and glutamate at EU positions 252, 254, and 256, respectively; and/or
the IgG Fc comprises the amino acids leucine and serine at EU positions 428 and 434, respectively.

76. The method of claim 74, wherein the IgG Fc comprises an amino acid sequence at least 85% identical to the amino acid sequence of a human IgG1 Fc.

77. The method of claim 74, wherein the IgG Fc comprises an amino acid sequence at least 95% identical to an amino acid sequence selected from the group consisting of SEQ ID NOs: 50-52 and 201-203.

78. The method of claim 74, wherein the IgG Fc is linked to the N-terminus of the first peptide or to the C-terminus of the second peptide.

79. The method of claim 66, wherein the fusion protein comprises an amino acid sequence selected from the group consisting of SEQ ID NOs: 53-118, 204-211, and 214-221.

80. The method of claim 66, wherein the first and second peptides do not comprise the amino acid sequence of a peptide selected from the group consisting of SEQ ID NOs:187-190.

81. The method of claim 66, wherein the fusion protein has a pI from 6.0 to 8.2.

82. The method of claim 66, wherein the polynucleotide is an RNA molecule or a DNA molecule.

83. The method of claim 66, wherein the polynucleotide is comprised within an expression vector, optionally wherein the expression vector is a plasmid or a viral vector.

84. The method of claim 66, wherein the host cell is prokaryotic cell, optionally wherein the prokaryotic cell is an E. coli cell or a Bacillus cell.

85. The method of claim 66, wherein the host cell is a eukaryotic cell, optionally wherein the eukaryotic cell is selected from the group consisting of a yeast cell, an insect cell, and a mammalian cell, optionally wherein the mammalian cell is selected from the group consisting of a CHO cell, a HeLa cell, and a 293 cell.


SEQUENCE LISTING (225 sequences)

SEQ ID NO: 1 - protein, 29 aa, synthetic construct
DSWMEEVIKLCGRELVRAQIAICGMSTWS

SEQ ID NO: 2 - protein, 24 aa, synthetic construct
QLYSALANKCCHVGCTKRSLARFC

SEQ ID NO: 3 - protein, 27 aa, synthetic construct
  VARIANT 4: note = can be methionine, lysine, or glutamine
  VARIANT 25: note = can be methionine or lysine
DSWXEEVIKLCGRELVRAQIAICGXST

SEQ ID NO: 4 - protein, 25 aa, synthetic construct
  VARIANT 1: note = can be arginine or absent (e.g., no amino acid)
  VARIANT 3: note = can be leucine or aspartic acid
  VARIANT 23: note = can be arginine, glutamine, or glutamate
XQXYSALANKCCHVGCTKRSLAXFC

SEQ ID NO: 5 - protein, 27 aa, synthetic construct
DSWKEEVIKLCGRELVRAQIAICGKST

SEQ ID NO: 6 - protein, 27 aa, synthetic construct
DSWMEEVIKLCGRELVRAQIAICGKST

SEQ ID NO: 7 - protein, 27 aa, synthetic construct
DSWQEEVIKLCGRELVRAQIAICGKST

SEQ ID NO: 8 - protein, 25 aa, synthetic construct
RQLYSALANKCCHVGCTKRSLARFC

SEQ ID NO: 9 - protein, 24 aa, synthetic construct
QLYSALANKCCHVGCTKRSLARFC

SEQ ID NO: 10 - protein, 25 aa, synthetic construct
RQLYSALANKCCHVGCTKRSLAQFC

SEQ ID NO: 11 - protein, 25 aa, synthetic construct
RQLYSALANKCCHVGCTKRSLAEFC

SEQ ID NO: 12 - protein, 25 aa, synthetic construct
RQDYSALANKCCHVGCTKRSLAQFC

SEQ ID NO: 13 - protein, 25 aa, synthetic construct
RQDYSALANKCCHVGCTKRSLARFC

SEQ ID NO: 14 - protein, 4 aa, synthetic construct
GGGE

SEQ ID NO: 15 - protein, 4 aa, synthetic construct
GEGE

SEQ ID NO: 16 - protein, 4 aa, synthetic construct
GGEG

SEQ ID NO: 17 - protein, 4 aa, synthetic construct
GEGG

SEQ ID NO: 18 - protein, 4 aa, synthetic construct
GGEE

SEQ ID NO: 19 - protein, 13 aa, synthetic construct
GGGEGGGEGGGEG

SEQ ID NO: 20 - protein, 15 aa, synthetic construct
GGGEGGGEGGGEGGG

SEQ ID NO: 21 - protein, 13 aa, synthetic construct
GEGGGEEGGGEGG

SEQ ID NO: 22 - protein, 13 aa, synthetic construct
GGGEEGGGEEGGG

SEQ ID NO: 23 - protein, 13 aa, synthetic construct
GGEGEGGEGEGGS

SEQ ID NO: 24 - protein, 12 aa, synthetic construct
GGGEGGGEGGGE

SEQ ID NO: 25 - protein, 65 aa, synthetic construct
DSWKEEVIKLCGRELVRAQIAICGKSTGGGEGGGEGGGEGRQLYSALANKCCHVGCTKRSLARFC

SEQ ID NO: 26 - protein, 66 aa, synthetic construct
DSWKEEVIKLCGRELVRAQIAICGKSTGGGEGGGEGGGEGGGQLYSALANKCCHVGCTKRSLARFC

SEQ ID NO: 27 - protein, 65 aa, synthetic construct
DSWMEEVIKLCGRELVRAQIAICGKSTGGGEGGGEGGGEGRQLYSALANKCCHVGCTKRSLARFC

SEQ ID NO: 28 - protein, 65 aa, synthetic construct
DSWQEEVIKLCGRELVRAQIAICGKSTGGGEGGGEGGGEGRQLYSALANKCCHVGCTKRSLARFC

SEQ ID NO: 29 - protein, 66 aa, synthetic construct
DSWMEEVIKLCGRELVRAQIAICGKSTGGGEGGGEGGGEGGGQLYSALANKCCHVGCTKRSLARFC

SEQ ID NO: 30 - protein, 66 aa, synthetic construct
DSWQEEVIKLCGRELVRAQIAICGKSTGGGEGGGEGGGEGGGQLYSALANKCCHVGCTKRSLARFC

SEQ ID NO: 31 - protein, 65 aa, synthetic construct
DSWMEEVIKLCGRELVRAQIAICGKSTGGEGGGEEGGGEGGQLYSALANKCCHVGCTKRSLARFC

SEQ ID NO: 32 - protein, 65 aa, synthetic construct
DSWQEEVIKLCGRELVRAQIAICGKSTGGEGGGEEGGGEGGQLYSALANKCCHVGCTKRSLARFC

SEQ ID NO: 33 - protein, 65 aa, synthetic construct; identical to SEQ ID NO: 31

SEQ ID NO: 34 - protein, 65 aa, synthetic construct; identical to SEQ ID NO: 32

SEQ ID NO: 35 - protein, 65 aa, synthetic construct
DSWQEEVIKLCGRELVRAQIAICGKSTGGGEGGGEGGGEGRQLYSALANKCCHVGCTKRSLAQFC

SEQ ID NO: 36 - protein, 65 aa, synthetic construct; identical to SEQ ID NO: 28

SEQ ID NO: 37 - protein, 65 aa, synthetic construct; identical to SEQ ID NO: 35

SEQ ID NO: 38 - protein, 65 aa, synthetic construct
DSWQEEVIKLCGRELVRAQIAICGKSTGGGEEGGGEEGGGRQLYSALANKCCHVGCTKRSLARFC

SEQ ID NO: 39 - protein, 65 aa, synthetic construct
DSWQEEVIKLCGRELVRAQIAICGKSTGGGEEGGGEEGGGRQLYSALANKCCHVGCTKRSLAQFC

SEQ ID NO: 40 - protein, 65 aa, synthetic construct; identical to SEQ ID NO: 38

SEQ ID NO: 41 - protein, 65 aa, synthetic construct; identical to SEQ ID NO: 39

SEQ ID NO: 42 - protein, 65 aa, synthetic construct
DSWQEEVIKLCGRELVRAQIAICGKSTGGGEGGGEGGGEGRQDYSALANKCCHVGCTKRSLAQFC

SEQ ID NO: 43 - protein, 65 aa, synthetic construct
DSWQEEVIKLCGRELVRAQIAICGKSTGGGEGGGEGGGEGRQDYSALANKCCHVGCTKRSLARFC

SEQ ID NO: 44 - protein, 65 aa, synthetic construct; identical to SEQ ID NO: 42

SEQ ID NO: 45 - protein, 65 aa, synthetic construct
DSWQEEVIKLCGRELVRAQIAICGKSTGGEGEGGEGEGGSRQLYSALANKCCHVGCTKRSLAQFC

SEQ ID NO: 46 - protein, 65 aa, synthetic construct
DSWQEEVIKLCGRELVRAQIAICGKSTGGEGEGGEGEGGSRQLYSALANKCCHVGCTKRSLARFC

SEQ ID NO: 47 - protein, 65 aa, synthetic construct; identical to SEQ ID NO: 45

SEQ ID NO: 48 - protein, 65 aa, synthetic construct
DSWQEEVIKLCGRELVRAQIAICGKSTGGEGEGGEGEGGSRQDYSALANKCCHVGCTKRSLAQFC

SEQ ID NO: 49 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 53 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGRQLYSALANKCCHVGCTKRSLARFC

SEQ ID NO: 54 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGGGQLYSALANKCCHVGCTKRSLARFC

SEQ ID NO: 55 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGRQLYSALANKCCHVGCTKRSLARFC

SEQ ID NO: 56 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLARFC

SEQ ID NO: 57 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLARFC

SEQ ID NO: 58 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGGGQLYSALANKCCHVGCTKRSLARFC

SEQ ID NO: 59 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGGGQLYSALANKCCHVGCTKRSLARFC

SEQ ID NO: 60 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGGGQLYSALANKCCHVGCTKRSLARFC

SEQ ID NO: 61 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGEGGGEEGGGEGGQLYSALANKCCHVGCTKRSLARFC

SEQ ID NO: 62 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGEGGGEEGGGEGGQLYSALANKCCHVGCTKRSLARFC

SEQ ID NO: 63 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWMEEVIKLCGRELVRAQIAICGKSTGGEGGGEEGGGEGGQLYSALANKCCHVGCTKRSLARFC

SEQ ID NO: 64 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGEGGGEEGGGEGGQLYSALANKCCHVGCTKRSLARFC

SEQ ID NO: 65 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLAQFC

SEQ ID NO: 66 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLAQFC

SEQ ID NO: 67 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLARFC

SEQ ID NO: 68 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLARFC

SEQ ID NO: 69 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLAQFC

SEQ ID NO: 70 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLAQFC

SEQ ID NO: 71 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLARFC

SEQ ID NO: 72 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLARFC

SEQ ID NO: 73 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLAQFC

SEQ ID NO: 74 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLAQFC

SEQ ID NO: 75 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLARFC

SEQ ID NO: 76 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLARFC

SEQ ID NO: 77 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLAQFC

SEQ ID NO: 78 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLAQFC

SEQ ID NO: 79 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQDYSALANKCCHVGCTKRSLAQFC

SEQ ID NO: 80 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQDYSALANKCCHVGCTKRSLARFC

SEQ ID NO: 81 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQDYSALANKCCHVGCTKRSLAQFC

SEQ ID NO: 82 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQLYSALANKCCHVGCTKRSLAQFC

SEQ ID NO: 83 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLARFC

SEQ ID NO: 84 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLAQFC

SEQ ID NO: 85 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQDYSALANKCCHVGCTKRSLAQFC

SEQ ID NO: 86 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGRQLYSALANKCCHVGCTKRSLARFC

SEQ ID NO: 87 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTGGGEGGGEGGGEGGGQLYSALANKCCHVGCTKRSLARFC

SEQ ID NO: 88 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGRQLYSALANKCCHVGCTKRSLARFC

SEQ ID NO: 89 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLARFC

SEQ ID NO: 90 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLARFC

SEQ ID NO: 91 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGGEGGGEGGGEGGGQLYSALANKCCHVGCTKRSLARFC

SEQ ID NO: 92 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGGGQLYSALANKCCHVGCTKRSLARFC

SEQ ID NO: 93 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGGGQLYSALANKCCHVGCTKRSLARFC

SEQ ID NO: 94 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWMEEVIKLCGRELVRAQIAICGKSTGGEGGGEEGGGEGGQLYSALANKCCHVGCTKRSLARFC

SEQ ID NO: 95 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGEGGGEEGGGEGGQLYSALANKCCHVGCTKRSLARFC

SEQ ID NO: 96 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWMEEVIKLCGRELVRAQIAICGKSTGGEGGGEEGGGEGGQLYSALANKCCHVGCTKRSLARFC

SEQ ID NO: 97 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGEGGGEEGGGEGGQLYSALANKCCHVGCTKRSLARFC

SEQ ID NO: 98 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEGGGEGGGEGRQLYSALANKCCHVGCTKRSLAQFC

SEQ ID NO: 99 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQLYSALANKCCHVGCTKRSLAQFC

SEQ ID NO: 100 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLARFC

SEQ ID NO: 101 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLARFC

SEQ ID NO: 102 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEGGGEGGGEGRQLYSALANKCCHVGCTKRSLAQFC

SEQ ID NO: 103 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQLYSALANKCCHVGCTKRSLAQFC

SEQ ID NO: 104 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLARFC

SEQ ID NO: 105 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLARFC

SEQ ID NO: 106 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWQEEVIKLCGRELVRAQIAICGKSTGGGEEGGGEEGGGRQLYSALANKCCHVGCTKRSLAQFC

SEQ ID NO: 107 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEEGGGEEGGGRQLYSALANKCCHVGCTKRSLAQFC

SEQ ID NO: 108 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLARFC

SEQ ID NO: 109 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLARFC

SEQ ID NO: 110 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGSDSWQEEVIKLCGRELVRAQIAICGKSTGGGEEGGGEEGGGRQLYSALANKCCHVGCTKRSLAQFC

SEQ ID NO: 111 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEEGGGEEGGGRQLYSALANKCCHVGCTKRSLAQFC

SEQ ID NO: 112 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGGEGGGEGGGEGRQDYSALANKCCHVGCTKRSLAQFC

SEQ ID NO: 113 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQDYSALANKCCHVGCTKRSLARFC

SEQ ID NO: 114 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGGEGGGEGGGEGRQDYSALANKCCHVGCTKRSLAQFC

SEQ ID NO: 115 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQLYSALANKCCHVGCTKRSLAQFC

SEQ ID NO: 116 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLARFC

SEQ ID NO: 117 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGEGGEGEGGSRQLYSALANKCCHVGCTKRSLAQFC

SEQ ID NO: 118 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGEGGEGEGGSRQDYSALANKCCHVGCTKRSLAQFC

SEQ ID NO: 119 - DNA, 885 bp, synthetic construct
  source 1..885: mol_type = other DNA
gataaaacacacacgtgtcccccctgcccggctccagaggcggctggtggtcccagcgtattcttgtttcctcccaaacctaaggatacgctcatgatatcccgcaccccagaagttacgtgtgtagtcgtcgacgtcagtcacgaagatccagaggtcaaatttaactggtatgtcgacggagtagaggtccacaatgcgaaaaccaagcccagagaagagcagtacaactccacgtatcgcgtcgtctccgtcctcaccgtactccatcaagattggctgaatgggaaagagtataaatgcaaagtatctaacaaggctctgccagctccgatagaaaagactatatcaaaggccaaggggcagccaagggagcctcaagtctatactttgcccccatctcgggatgagcttacgaaaaaccaggtcagccttacctgtcttgttaaaggtttttatccgagtgacatcgcagtggaatgggaatctaatggtcaacctgaaaacaattacaaaaccacaccgccagtattggacagcgatggtagtttttttctttactcaaaactgactgtagataaaagcagatggcagcagggcaatgtcttttcatgtagcgttatgcatgaggctcttcacaaccactatacccaaaagtcattgtctcttagtcccggaaagggcggaagtgattcttggaaggaggaggtaatcaagttgtgcgggcgagagttggtacgggcacagatcgcgatatgcggaaaatccacaggtgggggcgaaggaggaggtgagggtggaggtgaaggacgacagttgtattccgccttggcaaacaagtgttgccatgtgggttgcacaaaacgcagtcttgcccgcttctgt

SEQ ID NO: 120 - DNA, 888 bp, synthetic construct
  source 1..888: mol_type = other DNA
gataagacacatacatgccctccctgtccggctccagaggcagccgggggtccatcagtcttcctttttccgcctaaacctaaggatacactgatgatctctcgaacaccggaggtcacttgtgttgtcgttgacgtatcacatgaggatcccgaagtaaagttcaactggtatgtcgatggtgtggaggttcataatgctaaaactaaaccacgggaggagcaatataattccacatatagggtcgtgagcgtgttgacggtgcttcatcaagactggcttaatgggaaggaatataaatgcaaagtgtcaaataaagcacttcctgcgccaatcgagaaaacaattagtaaggcaaaggggcagccgcgagaacctcaggtgtacaccttgccgccttctagagacgagctcacaaagaaccaagtttccctgacttgcctcgttaaggggttttatccgtccgatatagccgtggagtgggagtcaaacggccaaccggaaaataattacaaaacgacacccccagtattggatagtgacggctcttttttcctttattctaagctgactgtggacaaaagccgctggcagcagggcaatgtcttttcatgcagcgtaatgcatgaagccctgcacaaccactacacgcaaaaatccctttccttgtcacccggcaagggcggctctgactcctggaaagaggaagttataaaactctgtggccgagaacttgttcgagctcaaatcgcgatttgtggtaagtcaacgggtgggggcgaaggtggaggcgagggtgggggagaaggaggaggccagttgtactcagctcttgcaaataagtgttgccacgttggttgtacgaagcggagccttgctcgcttctgc

SEQ ID NO: 121 - DNA, 885 bp, synthetic construct
  source 1..885: mol_type = other DNA
gacaaaacacatacttgtccgccttgcccggcacccgaagcggccggcggacccagtgtctttctcttcccacccaaaccgaaagacactctgatgatttccaggacgcctgaagtgacctgcgttgtagttgatgtatcacacgaggatcccgaggtcaagttcaattggtatgtagatggggtggaggtccataatgcaaagacgaagccacgggaggaacagtacaactctacgtacagagttgtcagtgttttgaccgtccttcatcaggattggctgaacggtaaagaatataaatgcaaggttagcaataaagctttgcccgcccctatagagaaaacgatcagtaaggcgaaggggcagcctagggaaccccaggtatataccttgccgccaagtcgagatgagctgacgaagaaccaagtgagtctgacatgcctcgtgaagggcttctatccgagcgatatcgctgtcgaatgggagagcaatgggcagcctgagaataactataaaacaacgccacccgtcctcgactccgatggctcattcttcctgtacagtaaacttacagtagataagagtagatggcagcagggtaacgtctttagttgctccgtgatgcacgaggcattgcacaatcattacactcaaaaatctctgtccctgagtccgggcaaaggcggttcagatagctggatggaggaggtcataaagctttgtggacgagaactcgttcgcgcccagatagctatttgtgggaaatcaaccgggggtggagaaggtggcggagaagggggaggcgaagggcgccaactgtattctgcattggctaataagtgctgtcacgtaggatgtacaaaaaggtctctggcgagattctgc

SEQ ID NO: 122 - DNA, 885 bp, synthetic construct
  source 1..885: mol_type = other DNA
gacaagacgcacacttgtccaccttgccctgcgccggaagctgctggaggccccagtgtctttttgttcccgcccaaaccgaaggacactttgatgataagtcgcacgcccgaggttacctgtgtggttgtcgatgtctcacacgaagatccggaggtgaagtttaattggtatgtagatggcgtggaggttcataacgccaaaacgaaacccagagaagaacaatataacagtacatatcgagtagtatccgttctcactgtcctgcatcaagactggttgaacgggaaggaatataagtgcaaggtgagcaataaagcactcccggccccaatcgaaaagaccatcagcaaagcgaaggggcaacctcgagaaccccaggtatatacgctcccccctagtcgggatgaacttactaaaaatcaggttagcctcacttgccttgttaaagggttctatcccagtgatattgccgtcgaatgggaatcaaacgggcagccggaaaataactacaagacaacccctcctgtgctcgatagcgatggctcttttttcctctacagcaaacttaccgttgataagagccggtggcaacaaggtaatgttttctcctgctccgttatgcatgaagcactccataaccattatacccaaaaaagcctgtcacttagtccgggtaaaggaggtagtgattcttggcaggaggaggtaatcaaactttgtgggagggagctggtacgagctcagattgctatatgtggaaaaagcacgggcggaggagaaggaggtggcgaaggcgggggtgaaggtcggcaactctactccgctctcgctaataagtgctgccacgtcgggtgtacgaagcgctccctggcgcgattctgc

SEQ ID NO: 123 - DNA, 885 bp, synthetic construct
  source 1..885: mol_type = other DNA
gataaaacgcacacgtgtccgccctgcccagcgcctgaagccgcaggcgggccgtccgtcttcctctttcctccaaaacccaaagacacacttatgatcagtaggaccccagaggtaacctgcgtcgtggtcgacgtttcccatgaagacccagaggtcaagttcaactggtacgtcgacggtgtcgaagtacataatgctaaaacgaagcctcgggaagagcagtacaactctacctaccgcgtcgtttccgtactcaccgtacttcaccaggactggcttaacggtaaagagtataaatgcaaagtatctaataaggctctcgccgcgccgattgagaagacaatttcaaaggccaaggggcagccgcgggagccccaagtgtataccttgcccccgtcccgagatgagctgactaaaaaccaagtaagcttgacttgcttggtcaaaggcttctacccttccgatatagctgtcgaatgggagtcaaatggccaaccagagaacaattataaaactacacccccggtcttggattctgatggctcattttttctctattctaaactgaccgtggataagtctcgctggcagcaaggtaacgtgttcagttgctctgttcttcacgaagcactgcacagtcattacactcagaagagtcttagcctgagccctggtaaagggggttctgattcctggcaggaggaagtaataaaactctgtggccgggagttggtacgggcgcagattgcgatatgcggtaagagcaccggcggaggcgaaggcggtggggaaggaggaggagaagggagacaactctattccgcattggcaaataagtgctgccacgtcgggtgtaccaaacgatcccttgcacggttctgt

SEQ ID NO: 124 - DNA, 888 bp, synthetic construct
  source 1..888: mol_type = other DNA
gataagacccatacgtgccccccttgccctgcgcctgaggcagcgggtggcccatcagtctttttgttcccgcccaagccaaaggacaccctcatgattagtagaacaccggaggttacgtgcgtcgtagtggatgtcagccacgaggatcccgaggttaagtttaactggtacgttgatggggttgaggtccataatgcgaagactaagccgagagaggaacagtacaattccacgtatagagttgtctctgtactgactgtgctgcatcaagattggcttaacggtaaggagtacaagtgcaaagtctctaataaggctcttcctgcacccattgagaaaactataagcaaagcaaaaggtcaacctcgcgaacctcaggtgtacacactgccaccctctagggacgagcttaccaaaaatcaagtatctcttacctgccttgtgaaagggttttatccctcagatattgcggttgagtgggagtctaacggacaacctgagaacaactataagactactcccccggtgcttgattcagacgggagtttttttttgtatagcaaacttaccgtcgacaaaagccggtggcaacagggcaatgtattcagttgttctgtaatgcatgaagctttgcataatcattacacccaaaagagtctttccctgtctcctggaaaaggggggtcagactcctggatggaggaggtgatcaaactgtgtgggagagagctcgtccgggctcagatagctatatgcggcaagtctacgggtgggggagagggcggaggagagggcggtggagaaggaggcggccaactctacagcgctctggccaataaatgttgtcatgtcgggtgtactaagcgctcactggcacgcttttgc

SEQ ID NO: 125 - DNA, 888 bp, synthetic construct
  source 1..888: mol_type = other DNA
gacaagacgcatacatgcccgccatgcccggcccccgaagctgctgggggaccatccgtattcctcttccctcccaaaccaaaagacacgttgatgataagtagaacaccagaggtaacgtgcgtggttgtcgatgtttcccacgaagatccggaggtaaaattcaattggtatgtagatggggtggaagtgcacaatgccaaaacaaagccgcgagaagaacaatacaatagtacttaccgggttgtgagcgtgctcacggtgttgcaccaagactggctcaacggcaaggaatacaagtgcaaagtatctaataaagctctgcctgcgccgatagagaagaccatcagtaaggccaaagggcagccccgagagccgcaagtttacactcttcctccgagcagagatgaattgaccaagaaccaagtaagtttgacgtgcctggtgaagggcttctacccctcagacattgcggtggagtgggaaagtaatggtcaaccggaaaacaactacaagaccacgccgcccgtcctcgactccgatgggtctttctttctttattcaaagttgacagtagataagtcaaggtggcagcaaggtaacgtgtttagttgtagtgtaatgcacgaggccctgcataatcattatacccaaaagagtttgagcctctcaccaggaaaaggcggatcagacagctggcaggaggaggtaattaaattgtgtggacgggagttggtcagggcgcaaatagccatctgcggtaagagcacgggtggaggagagggtggaggggaaggtgggggagaaggcggcgggcagctctattctgcactcgccaacaagtgttgtcacgtcggatgcacaaagagatctcttgctcgattctgc

SEQ ID NO: 126 - DNA, 888 bp, synthetic construct
  source 1..888: mol_type = other DNA
gacaaaacacacacctgtccgccttgcccggctcctgaagccgcgggtggccctagtgtgtttttgtttccgccgaaacctaaggataccctcatgataagccggacgcccgaggttacctgtgtcgtggtcgatgttagtcatgaggatccagaagtcaagtttaattggtacgtcgacggcgttgaagtccacaatgcaaaaactaaaccgcgagaagaacagtacaactccacctacagagttgtctcagttttgacagttctccatcaggattggctcaatggaaaggaatataagtgcaaggtcagcaataaagcgcttgccgcccctatagagaagaccattagcaaggcgaaaggacagccccgcgagccccaggtctatacgctgcctcctagcagagatgagctcacgaaaaatcaggtcagcttgacatgcttggtgaagggcttctaccccagtgacatcgcagttgaatgggagagcaacggccaacctgagaacaactacaaaacaacgcccccggttcttgacagcgatgggtccttctttctttactctaagcttacagttgataaaagcaggtggcagcaggggaatgtgttctcatgttccgtactgcatgaggctctgcattctcactacacccaaaaaagccttagcctgagccccggtaagggaggtagtgactcatggcaagaggaagtgattaagctctgcggccgggagttggtgagagcccaaatcgccatttgcggtaaaagtaccggagggggcgagggaggaggcgaaggtggaggtgaaggaggtggacagttgtactcagctcttgcaaataaatgttgtcatgttggttgcacgaaaagatctcttgcgaggttctgt

SEQ ID NO: 127 - DNA, 885 bp, synthetic construct
  source 1..885: mol_type = other DNA
gataagacgcatacttgtccaccgtgccccgcaccggaagcggctggtggtccatcagtttttctgttcccaccgaaacctaaggacacgttgatgatatcacggacaccagaggttacgtgcgtagtggtggatgtgagccacgaggatccagaagttaaatttaattggtacgtagatggagtggaggttcataatgcgaagacaaagcctcgcgaggaacagtataattccacctatcgcgtcgtatctgtgcttacggtacttcaccaagactggttgaacggtaaggaatataaatgcaaggtttccaataaagcacttcctgcgccaattgagaagacaatatccaaagctaaaggtcaacccagggaaccgcaagtctacactctccccccgtctcgcgatgaattgacgaagaaccaggttagtctcacctgcctggtcaaggggttttacccctctgacatagctgtagaatgggagtctaatggacagccagagaacaattacaaaacgacccccccggtcctcgattctgatgggagtttttttctttattcaaaattgactgtcgataagtcaagatggcaacagggtaacgtattttcttgcagtgttatgcatgaagcattgcacaaccactatacacaaaaatcattgagtttgagtcccggtaaagggggaagcgactcatggatggaagaagtaatcaagctgtgcgggcgagagcttgtgcgagctcagatagcaatctgtggtaagtctacaggtggagagggtggcggtgaagaaggcgggggagagggaggccagctttattctgccctggctaacaagtgctgtcacgttggatgcacgaagcgctccctggcccgattctgc

SEQ ID NO: 128 - DNA, 885 bp, synthetic construct
  source 1..885: mol_type = other DNA
gataagacgcatacttgtcccccatgtcccgctccggaagccgctggcggcccctccgtttttctgttcccgccgaaaccgaaagacaccctgatgatatcacgcactcccgaggtcacttgcgtggtagtcgatgttagtcatgaagatcctgaggtcaaattcaattggtatgtagatggcgttgaggtacacaacgcgaagacaaaaccccgagaagaacagtataactcaacctaccgcgtagtttcagttcttaccgtactgcaccaagactggttgaacggtaaagagtacaaatgtaaagtcagcaataaagctttgccagcacctatcgaaaaaaccatcagtaaggccaagggtcaacccagggagccgcaagtgtacactcttccccctagcagggatgaattgaccaagaatcaggtctctttgacgtgcctcgttaagggtttctatcccagcgatatagccgtagaatgggagtctaacggtcagccagaaaataactataagacaaccccgcctgttttggattccgacggctctttttttctctactctaagttgaccgttgataagagcagatggcagcagggaaacgtattttcttgttccgtgatgcacgaagccctgcacaatcactatacgcaaaagtctctgagcttgagtccgggtaaaggcggttctgactcctggcaggaggaagtcataaaactctgcggaagagagctcgtaagggcgcaaatcgctatttgtggtaagagcaccggtggggaaggaggcggtgaagagggtggcggcgagggtgggcaattgtattccgcgcttgccaataaatgttgtcacgtaggctgcacaaagcgaagtctcgctaggttctgc

SEQ ID NO: 129 - DNA, 882 bp, synthetic construct
  source 1..882: mol_type = other DNA
gacaagacccacacatgtcccccgtgtccggcaccagaagcagcggggggaccgtcagtattcttgtttccaccgaagcccaaagacacattgatgatttcacgaactcctgaagttacctgtgtggttgtagatgtatcacacgaagacccagaagtcaaattcaattggtatgtcgacggggttgaagttcacaatgcgaagacgaagccccgggaggaacagtacaacagcacgtacagggttgtgagcgttcttactgtattgcaccaggattggctcaacggcaaggagtataaatgtaaagtttctaataaggctcttcctgccccaattgaaaagacgatatctaaagcgaagggccaaccacgggaacctcaggtgtacacacttccgcctagcagggatgagttgaccaagaatcaagtctctttgacgtgcctggtcaaggggttttacccatcagatatcgccgtcgaatgggagtcaaacggacaacccgaaaataactataaaactactccaccagttctggatagcgacggctcattttttctgtattcaaagctcactgtagacaagtctaggtggcagcagggtaatgtcttctcctgctcagtaatgcatgaggctcttcacaaccactatactcaaaagagcctttccctgtcacctggcggtggaagcgactcatggatggaggaggtaataaagctctgcggaagagaactggtacgcgcacaaatcgcaatttgtggtaagagtactggcggggaaggaggtggggaagaagggggcggtgagggcggacagctctattctgcacttgcaaacaaatgttgccacgtgggatgtactaagcgaagccttgcaagattctgc

SEQ ID NO: 130 - DNA, 882 bp, synthetic construct
  source 1..882: mol_type = other DNA
gataaaacccacacatgccctccatgccctgctccagaggccgccggtgggccatcagttttcttgtttccgcctaaaccaaaggacacgcttatgatctccaggacccccgaagttacgtgtgtggtggttgatgttagtcacgaggacccggaagtcaagttcaactggtacgttgatggtgtagaggtgcacaatgcaaagacgaagccacgcgaagaacaatacaacagcacatatcgagttgtgagcgtactcacggtactgcatcaggactggctgaacggtaaagaatacaaatgtaaagtctccaataaggcacttcctgcgccgatagaaaaaacgatcagtaaggccaagggccaaccccgagaaccacaggtatatacgctcccaccgtcacgagacgagttgacaaaaaatcaggtctccctgacttgcctcgtgaaaggtttttatccctcagatattgctgttgagtgggaaagcaatgggcagccagagaataattataagacgactcctccggttttggattccgacggtagttttttcttgtatagtaagcttactgtagacaagtcaagatggcaacaaggtaatgtgttctcttgctcagttatgcatgaagctcttcataaccattacacgcaaaagagtctcagtctgagccccggtggcggtagcgacagttggcaggaagaggtgattaagttgtgcggtcgcgagctcgttcgggcccaaattgcaatctgcggaaaatctacgggcggagagggcgggggtgaggagggtgggggtgaaggtgggcagctctatagcgcccttgcgaataaatgttgtcacgtcggatgcacaaagaggtccctcgccaggttctgc

SEQ ID NO: 131 - DNA, 885 bp, synthetic construct
  source 1..885: mol_type = other DNA
gataagacccacacttgccccccttgccctgcccccgaagcggccggaggtccttcagtatttttgtttccaccgaaacccaaagatactttgatgatatcaagaactcctgaagtcacctgcgtggtagttgacgtatctcatgaggatcccgaggtgaagttcaattggtacgtcgatggcgtcgaggttcataacgctaagactaagccgagggaagagcaatataattccacttatagggtggtgtccgtcttgactgttttgcaccaggattggttgaacgggaaagagtacaaatgtaaggtgagtaataaagctttggctgctcccatcgaaaagacaataagcaaggccaaggggcaacctcgggagccgcaggtgtacacccttcctcccagtagagacgaactgacaaaaaaccaggtgtccctgacctgccttgtgaaggggttttacccgagcgacatagcggttgaatgggagagcaacgggcaacccgagaacaactacaaaactacaccgcctgtcctggactccgatggaagcttcttcctctactccaaactgaccgtggacaaaagcagatggcaacaaggaaacgtattctcatgctcagtaatgcacgaagcattgcacaatcactacacccaaaagtccctctcactctcccctggtaagggcggatcagactcatggcaagaggaggtaattaagttgtgcgggagggagctcgtccgcgcgcaaatagccatttgtggcaagtccactggaggaggcgagggtggaggagagggtggtggggagggcaggcaactctacagtgcgctcgccaataaatgctgccatgttgggtgcacgaagcgcagtctcgcacaattctgc

SEQ ID NO: 132 - DNA, 885 bp, synthetic construct
  source 1..885: mol_type = other DNA
gataagacccacacgtgtcctccatgtccggcaccggaggctgctggcgggccttctgtattcctcttcccacccaagccaaaagacacattgatgatatcaaggacgccggaagtcacctgtgttgttgtggacgtttcccatgaagacccagaggtaaaattcaattggtatgtggacggcgtagaggttcacaacgccaaaaccaaaccccgagaggaacagtataatagcacatatcgagtagtatctgttctcacagtgctccatcaagactggcttaatggtaaagagtataaatgcaaagtttccaataaagccctcgctgcaccgatcgagaagacaatcagtaaagcgaagggccagcctcgggaaccgcaggtgtatactcttccaccctcaagagacgagctcactaaaaaccaagtttcattgacatgcctcgtcaaaggtttctacccatcagacatcgcggtcgaatgggaaagtaatgggcagccggaaaacaactataaaacgacgccgcccgtcttggattctgatggttcattttttctttactctaaattgaccgtcgataaaagtaggtggcaacaaggaaatgttttttcctgctccgtcctgcatgaagcgttgcacagtcactatacccagaagagtctttctttgtcacccggaaaaggcggttcagattcatggcaggaagaagtaattaaactctgtggccgcgagcttgttagggcgcagatagccatatgtggtaaaagcaccggaggaggtgaaggcggaggcgaaggaggtggggaaggaagacaattgtattctgcacttgcaaataaatgctgtcatgtggggtgcacgaaacgcagtcttgcacaattttgt

SEQ ID NO: 133 - DNA, 882 bp, synthetic construct
  source 1..882: mol_type = other DNA
gacaaaacccatacctgccccccttgccctgcaccagaagcggcgggaggacctagcgtttttctttttcctccgaaaccgaaagataccctcatgatatcaagaacacctgaggttacttgcgttgtcgtggacgtgagtcacgaagaccccgaggtgaagttcaactggtatgtagatggagtggaggtccataatgcaaaaacgaaaccgagagaagaacaatacaactctacatatcgagtcgtgtcagtactcacggttttgcatcaagattggctgaacggtaaggagtacaagtgtaaggttagcaacaaggctctcgcggcgccgatagaaaagactataagtaaagcaaaaggccagcccagagaacctcaagtttacactctgcctcccagcagagatgaactgactaaaaatcaggtttcattgacctgtctcgtcaagggtttttatccaagcgacatagcagttgaatgggaaagcaacggtcaaccagaaaataattacaaaaccactccaccagtcttggactctgacggatccttctttctctattcaaaattgacggtggataaatctaggtggcagcaaggcaacgtcttctcttgtagcgttatgcatgaggcgctgcacaaccactacacacaaaagtctcttagtttgagcccgggcggcggaagcgactcttggcaagaggaagtgataaaactctgtggtcgagaattggtacgcgcgcagatcgctatctgcggcaagtccacagggggaggggaaggtggcggggaaggtggtggcgagggcaggcagttgtatagtgcacttgccaacaagtgctgccatgtggggtgcaccaagcgcagtttggcacggttctgc

SEQ ID NO: 134 - DNA, 882 bp, synthetic construct
  source 1..882: mol_type = other DNA
gataaaactcacacttgtcccccgtgtccggcaccagaagccgcaggagggccatctgtctttctttttcccccaaaacccaaggatacactgatgatctcccgcactcccgaagttacttgtgtcgtagtagacgtttctcacgaggacccagaggtgaaattcaattggtatgttgacggagtagaggtgcataatgccaagacaaagccccgagaggaacaatacaattcaacctacagagtagtgtccgttcttacggttctccatcaggattggctcaacggtaaggaatataagtgcaaggtaagcaacaaagcgctggccgcacccattgagaaaaccatttcaaaagctaaaggccaaccccgcgaaccacaagtttatactctccccccaagtcgcgatgaacttacaaaaaatcaagtctcattgacgtgcttggtcaaaggcttctacccgagcgatatcgctgttgaatgggagtctaatggacaaccggaaaataactataaaactacacccccagtcctcgattcagacggcagcttcttcctgtattcaaaactgacggttgacaaatcacgctggcaacagggtaacgttttttcctgtagcgttcttcatgaagccttgcacagtcactacacccagaagtcccttagcttgtcacctggcgggggttcagactcttggcaggaggaggtaatcaaactgtgcggaagagaactggtgagggctcagattgcaatttgtgggaagagcacgggtggcggtgaaggaggtggcgagggcggaggagaggggaggcaactctacagtgcgttggctaataaatgctgtcacgtcggctgtactaagagaagcctcgccagattttgc

SEQ ID NO: 135 - DNA, 882 bp, synthetic construct
  source 1..882: mol_type = other DNA
gacaagacgcatacttgccctccgtgccctgcaccagaagccgctggtggcccatctgtgtttttgttcccccctaagccaaaagacacattgatgatttcacgaactccagaagtgacttgcgtagttgttgacgtatcacacgaagaccccgaggttaaatttaattggtatgtggacggggtcgaggtgcataacgccaaaaccaaaccccgggaggaacaatataactctacgtatcgggtcgtatctgtgttgaccgtccttcaccaagattggttgaacggcaaggaatataagtgtaaagtgtctaataaagcattggctgccccgatagaaaagacgatctctaaagccaagggccaacccagagagcctcaagtatatactctcccaccgagtcgagatgagctcactaagaaccaggtgtcactcacgtgtctggttaaaggattttaccctagtgatatagccgtcgagtgggaatcaaatgggcagccggagaataactataagaccacgcctccagttctcgattccgatggtagctttttcctttactctaaacttacggtcgacaagtccaggtggcaacagggcaatgtattttcttgctccgtcatgcacgaggctttgcacaaccattacacgcaaaagtcactgtccctgtctcctggaggcggttctgacagttggcaggaggaggtaatcaaattgtgtgggcgggagttggttagggcgcaaattgctatttgcggcaaaagtactgggggcggtgaaggcggaggcgagggaggaggagaaggtcgacaactgtattctgccttggcgaacaaatgctgtcacgtcggctgtacgaaacggtctttggcccagttttgt

SEQ ID NO: 136 - DNA, 882 bp, synthetic construct
  source 1..882: mol_type = other DNA
gataagacacacacttgtccgccatgccctgcgccggaagcggcgggaggaccgtccgttttcctgttccctcccaaacccaaagacacgttgatgattagtcgcacgccagaagttacgtgcgttgtcgtagatgtatcccacgaagaccccgaggtgaagttcaattggtatgtagatggggtggaggtccataacgctaagaccaaaccacgcgaggaacaatataattctacgtaccgcgtagtgagcgttctcacagttcttcaccaggattggcttaacggcaaggagtataagtgtaaggtgtctaataaggccttggctgccccgatcgaaaaaacgataagtaaagcaaagggtcaacctagagaaccccaagtgtacactctcccgccatcacgggatgaattgactaagaaccaagtgtcactcacgtgtcttgtaaagggcttctacccatccgatatagccgttgagtgggaatccaatggtcagccagagaacaactataagacaactccgcccgtacttgatagtgacggttcctttttcctttacagtaaattgacggtagataagtctcgctggcagcaaggaaacgtcttttcttgttcagtgcttcatgaggcgcttcactcacactatactcagaagagtttgagtttgtctccaggtggaggcagcgactcatggcaagaggaagtaatcaaactgtgtggtcgcgaattggtacgagcacagatcgcgatctgcgggaaatcaacaggtggcggcgaaggcggcggggaaggcggcggcgaaggtaggcaactttactcagcccttgcgaacaaatgttgccacgtaggctgtactaagagaagtctcgcccagttttgc

SEQ ID NO: 137 - DNA, 885 bp, synthetic construct
  source 1..885: mol_type = other DNA
gacaagactcatacctgccccccttgtccagcaccagaagcagctggcgggccaagcgtgttcctgtttccacctaagcccaaagatacgttgatgatcagccgcaccccggaagtaacctgtgtagtagtagatgtgtcccacgaagaccccgaagtaaagtttaattggtacgtcgatggtgtcgaagtacataacgctaaaacgaagccccgagaagagcagtacaacagtacttacagagtagtttctgttcttacagtgctgcatcaggattggctgaacgggaaggagtataaatgtaaagtctcaaacaaggcacttgcggcaccaatagagaagacaatatctaaggccaaagggcagcctagagagccacaagtatatacgctgccccccagcagggacgagctgacaaagaaccaagtgtcactgacctgccttgttaagggcttctatccgagtgatattgctgttgaatgggaaagtaacggacagccggagaacaactataaaactactccacccgtgttggatagtgacggtagcttttttctgtactccaagttgacggtagacaaaagtcggtggcagcaggggaacgtattttcttgttctgtcatgcacgaagctcttcacaatcactatacgcagaagtccctctctctctctcctgggaagggtggttccgacagctggcaggaggaggtcattaaactgtgtggtagagagctggtacgggctcaaattgcaatttgtggtaagagtactggcggtggcgaggaagggggtggggaggagggcggaggtaggcagctctactctgctctcgccaacaagtgttgtcacgtcgggtgtactaaaagatcacttgcccgcttttgt

SEQ ID NO: 138 - DNA, 885 bp, synthetic construct
  source 1..885: mol_type = other DNA
gacaaaacacatacatgcccgccgtgtccggcgcctgaagcagcaggaggccccagtgtattccttttccctccaaagccaaaagatacgttgatgatatctaggacacctgaggttacctgcgtcgtagtggacgtatcccacgaagacccagaagtcaagtttaactggtatgtggacggagtggaggtacacaatgcaaagacaaagccgcgagaggaacaatataattccacctatagagtcgtgtcagtccttacggtcttgcaccaggactggctcaatggtaaggagtataagtgcaaagtatcaaacaaagctctcgcagcgcccatcgaaaagaccatcagcaaagctaagggccagccaagagagcctcaagtgtacacgttgccgccttcaagggacgagctcactaaaaatcaggtatcacttacgtgtcttgtcaaagggttttatccttccgacatcgcggttgaatgggagagcaatggacagccggagaataattataaaacgacgccgccggtccttgacagcgatggttcatttttcctttactcaaagctgacggttgataagtctaggtggcagcaggggaacgtcttttcctgtagtgtacttcatgaggcgctccattctcattacactcagaagtcactgagcctttcacccggcaaaggtggatcagactcctggcaagaagaggtaatcaaactctgtgggagggaactcgttcgagcccagattgcaatctgtgggaaaagcacaggcggaggggaagaagggggtggcgaagaaggtgggggcaggcagctctattcagctcttgccaacaaatgctgtcatgtaggctgcacaaagcgatcactggcgagattctgt

SEQ ID NO: 139 - DNA, 885 bp, synthetic construct
  source 1..885: mol_type = other DNA
gataaaactcatacttgcccaccctgcccggctcccgaggcagcaggtggaccctcagtatttttgttccctccgaaacctaaagatacacttatgattagccggacccctgaggtaacgtgtgtggtggttgacgtaagtcatgaagatccagaagtaaagtttaactggtacgtagacggtgtggaggtacataatgcgaagacaaaaccacgagaggaacagtataactctacctaccgcgtagtaagcgtacttactgtgctccaccaagactggcttaacgggaaagagtataagtgtaaagtcagtaataaagcactggccgccccgatcgaaaaaacaatcagcaaggccaaaggacaaccaagggagcctcaggtctatactcttcccccgagtagggatgagcttaccaagaaccaggtgtctctgacatgccttgtcaagggattttacccgagtgacatagccgtagaatgggagtcaaacggccaacctgaaaacaactataagaccacgcctcccgtactcgactcagatggaagctttttcctctatagcaagctgaccgtcgacaaaagtaggtggcaacagggaaacgtctttagttgttccgtcatgcacgaagctttgcataaccattacacccagaagagtctttccctttcccctggcaaggggggctccgactcctggcaagaggaagtaatcaaactgtgtgggcgcgagcttgtccgcgcgcaaatagccatttgcggaaaaagtactggaggaggagaggaaggcggcggcgaggaaggtgggggcaggcagctgtacagtgccttggctaacaagtgctgccatgtcggctgtacgaaaaggtctcttgctcaattctgt

SEQ ID NO: 140 - DNA, 885 bp, synthetic construct
  source 1..885: mol_type = other DNA
gataagacacatacctgtccaccctgcccagcacctgaagctgcaggcggccccagcgtattcctgtttcctccgaagccgaaagacacacttatgatttcccggacgcctgaggtaacttgcgtcgtagtagatgtgtctcacgaagaccccgaggtgaaattcaactggtacgttgatggtgtggaagttcataatgcgaaaactaaaccacgagaggagcaatataactcaacttatagagttgtgagcgtcttgacggtactgcaccaggactggctgaatggcaaagagtacaaatgcaaagtctcaaataaggcgttggcggctcccatagagaaaactatcagcaaagccaagggtcaacctcgggagccacaagtgtatactcttccgcctagtcgcgacgagctcacaaagaatcaggtgagtcttacttgtttggttaagggtttctaccccagtgacattgcggtcgagtgggaaagtaacggacagcctgaaaacaactataaaacaacgcctccagtactcgattcagatggttcattctttctttattccaaactcacagtcgacaagagtagatggcaacaagggaacgtgtttagctgtagcgtactccatgaggcactccactctcactatacccaaaagtctctcagcttgtcacccggaaaaggcggttctgacagttggcaagaggaagtgattaaattgtgtgggcgggaacttgtgagggctcaaatcgcgatttgcggcaagtccactggtggcggcgaggaaggaggaggtgaagaaggaggaggtaggcaactgtattcagcgttggcgaataaatgctgccatgttggatgtactaaacggagccttgctcagttctgc

SEQ ID NO: 141 - DNA, 882 bp, synthetic construct
  source 1..882: mol_type = other DNA
gataaaacgcatacttgccctccttgcccggcacctgaagctgccggaggtccttccgtgttcctgttcccacctaagccaaaagacacacttatgatttctcgcacaccagaagtaacgtgcgtcgtagttgacgtctcccatgaagacccggaggtaaaatttaattggtacgtcgacggggtagaagttcataacgcaaagactaaaccacgagaagagcaatacaactctacatacagagtagtaagcgttctcaccgttcttcatcaagattggctcaacggaaaggagtataagtgtaaggtgtccaataaagcgttggccgcaccaatcgaaaagaccataagcaaagccaaaggccaaccccgcgaaccgcaggtgtacacacttcccccgtccagggatgaattgacaaaaaaccaagtttccctcacgtgtctcgtcaagggattctacccgagtgatatcgcagttgaatgggaaagcaatggtcagcccgagaataactacaagactactccccctgtgttggactcagacggctcattcttcctctacagtaagttgactgtggacaaaagtcggtggcagcaaggcaatgtcttcagttgtagtgtaatgcatgaagcactccacaatcattacacccaaaaatccctgagcctgtccccgggcggaggttcagattcatggcaggaggaagttataaaactgtgcgggcgcgagttggtgagggcgcagatcgcaatctgtggaaagagtacgggaggtggcgaagagggtggtggagaagagggaggaggtcgacaactgtattccgcgctcgcgaacaagtgttgccacgttggctgcaccaaacgaagcctggctcgattttgc

SEQ ID NO: 142 - DNA, 882 bp, synthetic construct
  source 1..882: mol_type = other DNA
gacaagacacacacttgtccaccttgcccggctcccgaggcggcaggaggaccaagcgtttttctgttccctcccaaaccaaaggatacgcttatgatctctcgaacgccggaagttacttgcgtagtagttgatgtctcccatgaagatcccgaagtgaagttcaactggtatgtagatggtgtggaagttcataacgcgaaaaccaaaccacgcgaagaacagtataacagtacttatcgggttgtttcagtactcacggtgctccatcaagactggcttaatggaaaggagtataaatgtaaggtaagtaacaaggcattggcggctcccatcgagaagacaatctccaaagcaaaagggcaaccacgggagcctcaggtgtatacgttgccgcccagcagagatgaacttactaagaatcaggtgagtctcacttgtctcgtcaagggcttctatcccagcgatatagccgtagaatgggagagtaacggtcagccggagaacaactacaaaacaaccccgcctgttttggactccgatgggagtttttttctctacagcaaactcacggtagacaaaagcaggtggcagcagggcaatgttttcagttgctctgttctccacgaagccctccactcccactatactcagaagtctctgagtctctcaccagggggaggtagcgatagctggcaggaggaagtgatcaagttgtgcgggcgcgaactcgtgcgggcacaaattgctatatgcggtaaaagtacgggaggtggagaggagggtggaggtgaagaaggcggtggtagacaattgtatagtgcgctcgccaacaagtgttgtcatgtcgggtgtacgaaacggtccttggcgcggttttgc

SEQ ID NO: 143 - DNA, 882 bp, synthetic construct
  source 1..882: mol_type = other DNA
gacaagacacatacttgtccaccatgtcccgccccagaagctgcgggaggaccatcagtttttttgttccccccgaaaccgaaggataccctcatgataagtcgaacgcccgaagtcacttgcgtggtggttgatgttagccacgaggacccagaagtgaagttcaactggtacgtggacggggtcgaagttcataatgcgaaaacaaagcctcgcgaggaacagtacaactctacatacagggttgtgtctgttttgacagtcttgcaccaagattggctcaacgggaaggaatataagtgtaaggtaagcaataaagcactggcggccccgatcgaaaaaacgatatccaaggccaagggccagccccgagagcctcaggtatatactctgccgccaagccgggatgaactgactaaaaaccaggtctctttgacttgtcttgtcaagggattttacccaagtgacattgcggtagagtgggaaagcaacggtcaaccagaaaacaattacaagacgacaccgccggtactcgactcagatggatcctttttcctgtatagcaagctgacagtggacaagtcccggtggcagcaagggaacgtattttcatgcagcgtgatgcatgaggctcttcacaaccattacacacagaaaagtctgtcattgagccctggcggcgggagcgattcttggcaagaagaagttataaaactttgcggtcgagagctggttcgggcacaaattgctatctgcggaaaatctacaggaggaggcgaggagggagggggcgaagaaggcggggggagacagttgtacagtgcgctcgctaacaagtgttgccacgtcggttgcacaaagagatccctggctcaattctgt

SEQ ID NO: 144 - DNA, 882 bp, synthetic construct
  source 1..882: mol_type = other DNA
gataaaactcacacctgtcccccgtgtcccgcaccagaagcggccggtggtccctccgtttttctcttccctcctaaacctaaggacacacttatgattagcagaactccagaagttacgtgcgtagtcgttgacgttagtcatgaagatcctgaggttaagttcaactggtacgtagacggagtagaggtccacaacgccaagacgaaaccccgagaagagcagtataattctacctatcgagttgtttcagtattgacggtgcttcaccaagattggctgaatggcaaagagtataagtgcaaggtaagcaacaaagcactcgcggctcctatcgagaaaactatttccaaagctaagggccagcctcgcgaaccacaagtctataccctgccaccgagtcgggacgaactcaccaagaaccaagtgtctcttacttgcctcgttaaaggtttttatcccagcgacatagccgtcgaatgggagtccaatggccaacctgagaacaactataaaactacccctcctgtacttgatagcgacggaagttttttcctctattcaaaactcacagttgataagtctcgatggcaacagggcaacgtcttctcttgcagtgtgttgcatgaagctctgcactctcattacacacagaagagtttgtctctcagtccaggtggcggctcagatagctggcaggaagaagtaatcaagttgtgcggcagggaactggtaagggcacagatagccatttgtggaaaatctacgggtggcggtgaggaaggcggcggagaagaagggggaggtcggcagctgtatagtgcactcgcaaacaagtgctgccatgtcgggtgcaccaagcgatcccttgcccagttttgc

SEQ ID NO: 145 - DNA, 885 bp, synthetic construct
  source 1..885: mol_type = other DNA
gacaaaacgcacacctgtccaccgtgtcctgctccagaggcggccgggggaccgtccgttttcctttttcctcccaaacctaaggatacccttatgatctctcgcacgcccgaggttacctgtgttgtggttgacgtgtcccatgaagacccggaagtaaaatttaattggtacgtggacggggtcgaggttcataacgcaaagaccaagccacgagaggagcaatataattccacctatcgcgtagtctccgtcctcaccgtgcttcaccaggattggctcaacgggaaggaatacaaatgtaaagtcagtaataaggctttggcggccccgattgagaagactataagtaaggctaagggacagccacgagaaccgcaagtttatacattgcccccctctagggatgagttgactaagaatcaggtgtcactcacttgtctggtaaaagggttctacccgtccgacatcgctgtggaatgggaaagcaatgggcaacctgaaaataattataagacaacccctccggtgcttgatagcgacggatcattctttctctattccaagcttactgtagataagagtcgatggcaacaggggaacgtattcagttgctctgttctccatgaggccctgcatagtcactacacccaaaaaagccttagtttgagtcccgggaaaggaggctccgattcttggcaagaagaggtaataaagctgtgtggacgagaacttgtccgagcacaaattgcgatttgtggcaaatctacaggagggggagaaggaggcggcgaagggggaggcgagggcaggcaggattattccgctctggcgaacaaatgttgccatgttggatgcacgaaacgaagcctggctcagttttgc

SEQ ID NO: 146 - DNA, 882 bp, synthetic construct
  source 1..882: mol_type = other DNA
gataaaacgcatacctgcccaccgtgtcctgcacctgaggccgctggaggaccttccgtcttcctctttccacccaagccgaaagacacactcatgattagtagaactccagaggtcacgtgtgttgtggtggacgtcagtcacgaggaccctgaggttaagttcaactggtacgttgatggcgtagaagtccacaatgcaaagaccaaaccgagagaggagcaatataacagtacatatagggttgttagcgtacttactgttttgcatcaagactggttgaatgggaaggaatataaatgtaaagtctccaacaaggctctggctgcaccaatagaaaaaactatttctaaggcaaagggtcagcctagagagcctcaagtctataccttgccaccgtcaagagacgagctcactaaaaatcaggtgagcctgacctgtcttgtgaagggcttttacccgtcagatattgccgtggagtgggaatcaaacggtcagccggagaataactacaagacgaccccaccagtactcgatagcgatgggtctttctttctgtactccaagctcaccgtggacaaatcacgctggcaacagggcaacgtctttagttgcagcgtactgcacgaggcactgcacagccactacacacaaaagagtctttctctgtctcccggtggtggctccgatagttggcaggaagaagtcataaagctttgtggaagagagcttgtacgagcgcagattgcaatctgcgggaagagcactggaggaggtgagggagggggtgagggcgggggcgaaggacgccaggactattcagcacttgcaaacaaatgctgccatgtagggtgtacgaagcgctcactggcccggttttgc

SEQ ID NO: 147 - DNA, 882 bp, synthetic construct
  source 1..882: mol_type = other DNA
gataaaacacatacctgccccccatgcccagcccccgaagctgcaggggggccctctgttttcctttttccacccaaacctaaagatactctgatgattagtcggactccggaagtgacttgcgtcgttgtcgacgtctctcatgaggatccagaagttaagtttaactggtatgtcgacggggttgaggttcataatgcaaaaactaaaccgagagaagaacagtacaactctacttatagggttgtcagtgtactgaccgtcctgcaccaggattggcttaacggtaaggagtataagtgtaaagtgtccaataaagcccttgccgcacccatcgagaaaaccatctccaaggcaaaaggacagccaagggaaccgcaggtatatacacttccgccaagccgagacgaacttacgaagaaccaggtgtctctcacgtgtctcgtaaaagggttttatcccagcgatatcgcagttgagtgggagagcaatgggcagccagagaataattataagacaacccctcccgtgctggattcagacgggagtttttttctttactctaagctgaccgtagacaaaagtcgatggcagcaaggcaacgtcttttcctgctccgttctccatgaagcactgcatagccattatacccagaagtcactgagcctctctccagggggcgggtccgattcatggcaggaagaggtaatcaaactctgtggacgcgaactggttcgcgcgcagatagcgatttgcggcaaaagcacaggcggtggggaaggcggtggcgagggcggtggtgaaggtcgacaagattattctgctctcgctaacaagtgttgtcatgtaggatgtactaaaaggagtcttgcgcagttctgt

SEQ ID NO: 148 - DNA, 885 bp, synthetic construct
  source 1..885: mol_type = other DNA
gataagacgcacacatgcccaccctgtcctgcgcctgaagccgcggggggacccagcgtttttctcttcccgccgaaaccgaaagacacacttatgatcagccggactcccgaggttacctgcgtggtggtagatgtatctcacgaggatcccgaggtcaaattcaactggtacgttgatggggttgaagttcataatgccaaaacgaagccaagagaagagcagtataactccacatatagagttgtttccgtcttgactgttcttcaccaagattggctgaatgggaaggagtacaaatgtaaagttagcaacaaggcactcgccgctcccattgaaaaaactataagcaaagctaagggccaaccgcgcgaaccacaggtctacacgttgccgccctctagggacgaactcacgaagaatcaggtttcccttacctgcctcgttaaaggattctacccctctgacatagcggttgaatgggagagcaacggtcagcctgagaacaactacaaaacgacgcctccggtgttggattccgacggtagttttttcctctatagtaagctgacagtggataaatctcggtggcagcaagggaatgtattctcctgttcagtcctgcatgaagccctccactcccattatacacagaaatctctttctctgagtcccggtaaaggtgggagtgactcttggcaggaagaggtaattaagttgtgtggaagggagctggtaagagcacagattgccatctgtggcaaatccacgggcggcgaaggtgaggggggtgagggggaaggggggtccagacaactgtattctgctctggcgaataagtgttgccatgtagggtgcactaaacggtccttggcgcagttctgt

SEQ ID NO: 149 - DNA, 882 bp, synthetic construct
  source 1..882: mol_type = other DNA
gataaaactcatacgtgcccaccttgccccgcaccggaggctgctggaggaccctctgtcttcctgttcccgccgaagcctaaagacacattgatgatcagtcgaacaccggaagtcacctgtgtagtggttgatgtgagccatgaggaccctgaagtaaaatttaactggtatgttgatggcgtagaagtacacaacgcgaagactaaaccaagggaagagcaatacaactctacctatagggtcgttagcgtactgactgtgcttcaccaagactggcttaacgggaaggagtacaagtgcaaagtgagcaataaggccctcgccgcgcctatcgagaaaaccatttccaaagccaagggtcaaccaagggagcctcaggtttacaccctgcccccttcaagggatgagttgacaaaaaaccaggtaagtctgacgtgtctcgttaagggattctacccgtcagatatcgcggtagagtgggagagcaacggtcagccagaaaataattacaaaacaacacctccagttttggactctgatgggagtttttttctttattctaagttgacagtggataagtcacgctggcaacaggggaacgtatttagctgctcagtacttcatgaagcgttgcattctcactacacacagaagagcctctccttgagtcccggaggtggctctgattcttggcaggaggaggtaataaaactttgtggtagagaactggttcgcgctcagatagctatttgtggaaaatccactggcggtgaaggtgaaggtggagaaggagagggcggaagccggcagttgtactctgccctggctaataagtgctgtcacgtgggctgcactaagcggagcttggcaagattttgc

SEQ ID NO: 150 - DNA, 882 bp, synthetic construct
  source 1..882: mol_type = other DNA
gataaaactcatacctgtccaccttgtcctgcgcctgaggcagctggagggcctagcgtgttcctgttcccccccaaacccaaagacacgctcatgattagccgaacccctgaagtgacctgcgttgttgtggacgtaagccacgaagaccccgaagttaagtttaattggtacgtcgacggtgttgaggttcataacgcgaagactaagccgagagaggagcaatataacagcacctaccgcgtagtctcagttcttaccgtgctccaccaggactggcttaacgggaaggaatacaaatgcaaagtttccaacaaagccttggcagccccaatagagaagacaatatctaaggcgaaaggccaaccgcgggaaccgcaagtttataccctcccaccgagcagggatgagctgacaaaaaatcaggtttccctcacttgtctggtcaagggattttatccttcagacatagccgttgaatgggagagtaatgggcagccggagaataattacaagaccacccccccggtgttggacagcgacggttccttctttctctattctaaacttaccgtcgacaaatcacggtggcaacaaggaaatgtattctcatgcagtgtattgcacgaagctctgcactctcattacacccaaaaatccctctctctcagccctggcggtggatctgattcttggcaggaagaggtgattaaactgtgtgggcgagagcttgtccgagctcagatcgctatttgtggcaagagtaccggaggcgagggtgagggaggcgaaggcgagggcggaagccggcaactctatagcgcactcgctaataaatgttgtcatgtcggctgcacgaagcgctcactggcgcagttctgc

SEQ ID NO: 151 - DNA, 885 bp, synthetic construct
  source 1..885: mol_type = other DNA
gacaaaacgcatacctgtcctccatgccccgctcccgaggctgccggcggaccaagcgtatttctcttcccccctaaacctaaagacacattgatgataagtaggacgcctgaagtaacgtgtgttgtcgttgatgtaagccatgaagatcctgaagtaaagtttaattggtatgttgatggcgtagaagtacataacgctaagacgaagccacgggaagagcagtataactcaacttaccgcgttgtaagcgtgcttaccgtcctgcatcaggattggctgaatggtaaggaatataagtgcaaagtaagcaacaaagcattggccgcaccaatagagaagacgattagtaaagcaaaaggccagcccagagagccgcaggtttatacacttccaccaagcagagatgaacttacgaagaaccaggtgtctctgacttgtctggtcaagggtttctatccttccgacattgcagtggagtgggaaagcaatgggcagcccgaaaacaattataagacgacacctccagtgttggactcagacggttcctttttcttgtattccaaacttacagtggataagtcaaggtggcagcaaggcaacgtattttcttgtagtgttttgcacgaagccctgcattcccactatactcaaaagagcctcagtctgtccccaggaaagggagggagtgacagttggcaagaggaggtaataaaattgtgtggcagagagcttgtgcgcgctcagatcgcaatatgcgggaaatctactgggggtgagggtgagggcggcgagggagaggggggcagtcgccaagattattccgcccttgcgaataagtgttgtcacgtcggatgtactaagagatcattggctcagttttgt

SEQ ID NO: 152 - DNA, 945 bp, synthetic construct
  source 1..945: mol_type = other DNA
atggagacggacactttgctgctttgggtactgctgctttgggttcctggatctactggcgataaaacacacacgtgtcccccctgcccggctccagaggcggctggtggtcccagcgtattcttgtttcctcccaaacctaaggatacgctcatgatatcccgcaccccagaagttacgtgtgtagtcgtcgacgtcagtcacgaagatccagaggtcaaatttaactggtatgtcgacggagtagaggtccacaatgcgaaaaccaagcccagagaagagcagtacaactccacgtatcgcgtcgtctccgtcctcaccgtactccatcaagattggctgaatgggaaagagtataaatgcaaagtatctaacaaggctctgccagctccgatagaaaagactatatcaaaggccaaggggcagccaagggagcctcaagtctatactttgcccccatctcgggatgagcttacgaaaaaccaggtcagccttacctgtcttgttaaaggtttttatccgagtgacatcgcagtggaatgggaatctaatggtcaacctgaaaacaattacaaaaccacaccgccagtattggacagcgatggtagtttttttctttactcaaaactgactgtagataaaagcagatggcagcagggcaatgtcttttcatgtagcgttatgcatgaggctcttcacaaccactatacccaaaagtcattgtctcttagtcccggaaagggcggaagtgattcttggaaggaggaggtaatcaagttgtgcgggcgagagttggtacgggcacagatcgcgatatgcggaaaatccacaggtgggggcgaaggaggaggtgagggtggaggtgaaggacgacagttgtattccgccttggcaaacaagtgttgccatgtgggttgcacaaaacgcagtcttgcccgcttctgt

SEQ ID NO: 153 - DNA, 948 bp, synthetic construct
  source 1..948: mol_type = other DNA
atggagaccgatacgctgttgctgtgggtattgcttctctgggtgcccggctcaactggggataagacacatacatgccctccctgtccggctccagaggcagccgggggtccatcagtcttcctttttccgcctaaacctaaggatacactgatgatctctcgaacaccggaggtcacttgtgttgtcgttgacgtatcacatgaggatcccgaagtaaagttcaactggtatgtcgatggtgtggaggttcataatgctaaaactaaaccacgggaggagcaatataattccacatatagggtcgtgagcgtgttgacggtgcttcatcaagactggcttaatgggaaggaatataaatgcaaagtgtcaaataaagcacttcctgcgccaatcgagaaaacaattagtaaggcaaaggggcagccgcgagaacctcaggtgtacaccttgccgccttctagagacgagctcacaaagaaccaagtttccctgacttgcctcgttaaggggttttatccgtccgatatagccgtggagtgggagtcaaacggccaaccggaaaataattacaaaacgacacccccagtattggatagtgacggctcttttttcctttattctaagctgactgtggacaaaagccgctggcagcagggcaatgtcttttcatgcagcgtaatgcatgaagccctgcacaaccactacacgcaaaaatccctttccttgtcacccggcaagggcggctctgactcctggaaagaggaagttataaaactctgtggccgagaacttgttcgagctcaaatcgcgatttgtggtaagtcaacgggtgggggcgaaggtggaggcgagggtgggggagaaggaggaggccagttgtactcagctcttgcaaataagtgttgccacgttggttgtacgaagcggagccttgctcgcttctgc

SEQ ID NO: 154 - DNA, 945 bp, synthetic construct
  source 1..945: mol_type = other DNA
atggaaactgatactcttctgctgtgggtcctgctgctgtgggttccaggatctactggagacaaaacacatacttgtccgccttgcccggcacccgaagcggccggcggacccagtgtctttctcttcccacccaaaccgaaagacactctgatgatttccaggacgcctgaagtgacctgcgttgtagttgatgtatcacacgaggatcccgaggtcaagttcaattggtatgtagatggggtggaggtccataatgcaaagacgaagccacgggaggaacagtacaactctacgtacagagttgtcagtgttttgaccgtccttcatcaggattggctgaacggtaaagaatataaatgcaaggttagcaataaagctttgcccgcccctatagagaaaacgatcagtaaggcgaaggggcagcctagggaaccccaggtatataccttgccgccaagtcgagatgagctgacgaagaaccaagtgagtctgacatgcctcgtgaagggcttctatccgagcgatatcgctgtcgaatgggagagcaatgggcagcctgagaataactataaaacaacgccacccgtcctcgactccgatggctcattcttcctgtacagtaaacttacagtagataagagtagatggcagcagggtaacgtctttagttgctccgtgatgcacgaggcattgcacaatcattacactcaaaaatctctgtccctgagtccgggcaaaggcggttcagatagctggatggaggaggtcataaagctttgtggacgagaactcgttcgcgcccagatagctatttgtgggaaatcaaccgggggtggagaaggtggcggagaagggggaggcgaagggcgccaactgtattctgcattggctaataagtgctgtcacgtaggatgtacaaaaaggtctctggcgagattctgc

SEQ ID NO: 155 - DNA, 945 bp, synthetic construct
  source 1..945: mol_type = other DNA
atggagaccgacaccctcttgttgtgggttctcctcttgtgggtgcccggcagtactggagacaagacgcacacttgtccaccttgccctgcgccggaagctgctggaggccccagtgtctttttgttcccgcccaaaccgaaggacactttgatgataagtcgcacgcccgaggttacctgtgtggttgtcgatgtctcacacgaagatccggaggtgaagtttaattggtatgtagatggcgtggaggttcataacgccaaaacgaaacccagagaagaacaatataacagtacatatcgagtagtatccgttctcactgtcctgcatcaagactggttgaacgggaaggaatataagtgcaaggtgagcaataaagcactcccggccccaatcgaaaagaccatcagcaaagcgaaggggcaacctcgagaaccccaggtatatacgctcccccctagtcgggatgaacttactaaaaatcaggttagcctcacttgccttgttaaagggttctatcccagtgatattgccgtcgaatgggaatcaaacgggcagccggaaaataactacaagacaacccctcctgtgctcgatagcgatggctcttttttcctctacagcaaacttaccgttgataagagccggtggcaacaaggtaatgttttctcctgctccgttatgcatgaagcactccataaccattatacccaaaaaagcctgtcacttagtccgggtaaaggaggtagtgattcttggcaggaggaggtaatcaaactttgtgggagggagctggtacgagctcagattgctatatgtggaaaaagcacgggcggaggagaaggaggtggcgaaggcgggggtgaaggtcggcaactctactccgctctcgctaataagtgctgccacgtcgggtgtacgaagcgctccctggcgcgattctgc

SEQ ID NO: 156 - DNA, 945 bp, synthetic construct
  source 1..945: mol_type = other DNA
atggaaacagataccctcctcctctgggtccttcttctttgggtgcctggctcaactggagataaaacgcacacgtgtccgccctgcccagcgcctgaagccgcaggcgggccgtccgtcttcctctttcctccaaaacccaaagacacacttatgatcagtaggaccccagaggtaacctgcgtcgtggtcgacgtttcccatgaagacccagaggtcaagttcaactggtacgtcgacggtgtcgaagtacataatgctaaaacgaagcctcgggaagagcagtacaactctacctaccgcgtcgtttccgtactcaccgtacttcaccaggactggcttaacggtaaagagtataaatgcaaagtatctaataaggctctcgccgcgccgattgagaagacaatttcaaaggccaaggggcagccgcgggagccccaagtgtataccttgcccccgtcccgagatgagctgactaaaaaccaagtaagcttgacttgcttggtcaaaggcttctacccttccgatatagctgtcgaatgggagtcaaatggccaaccagagaacaattataaaactacacccccggtcttggattctgatggctcattttttctctattctaaactgaccgtggataagtctcgctggcagcaaggtaacgtgttcagttgctctgttcttcacgaagcactgcacagtcattacactcagaagagtcttagcctgagccctggtaaagggggttctgattcctggcaggaggaagtaataaaactctgtggccgggagttggtacgggcgcagattgcgatatgcggtaagagcaccggcggaggcgaaggcggtggggaaggaggaggagaagggagacaactctattccgcattggcaaataagtgctgccacgtcgggtgtaccaaacgatcccttgcacggttctgt

SEQ ID NO: 157 - DNA, 948 bp, synthetic construct
  source 1..948: mol_type = other DNA
atggagacggacaccctccttctctgggttttgctcctttgggtccctggttccactggagataagacccatacgtgccccccttgccctgcgcctgaggcagcgggtggcccatcagtctttttgttcccgcccaagccaaaggacaccctcatgattagtagaacaccggaggttacgtgcgtcgtagtggatgtcagccacgaggatcccgaggttaagtttaactggtacgttgatggggttgaggtccataatgcgaagactaagccgagagaggaacagtacaattccacgtatagagttgtctctgtactgactgtgctgcatcaagattggcttaacggtaaggagtacaagtgcaaagtctctaataaggctcttcctgcacccattgagaaaactataagcaaagcaaaaggtcaacctcgcgaacctcaggtgtacacactgccaccctctagggacgagcttaccaaaaatcaagtatctcttacctgccttgtgaaagggttttatccctcagatattgcggttgagtgggagtctaacggacaacctgagaacaactataagactactcccccggtgcttgattcagacgggagtttttttttgtatagcaaacttaccgtcgacaaaagccggtggcaacagggcaatgtattcagttgttctgtaatgcatgaagctttgcataatcattacacccaaaagagtctttccctgtctcctggaaaaggggggtcagactcctggatggaggaggtgatcaaactgtgtgggagagagctcgtccgggctcagatagctatatgcggcaagtctacgggtgggggagagggcggaggagagggcggtggagaaggaggcggccaactctacagcgctctggccaataaatgttgtcatgtcgggtgtactaagcgctcactggcacgcttttgc

SEQ ID NO: 158 - DNA, 948 bp, synthetic construct
  source 1..948: mol_type = other DNA
atggaaaccgacacccttttgttgtgggtattgctgttgtgggttcccggtagcacgggggacaagacgcatacatgcccgccatgcccggcccccgaagctgctgggggaccatccgtattcctcttccctcccaaaccaaaagacacgttgatgataagtagaacaccagaggtaacgtgcgtggttgtcgatgtttcccacgaagatccggaggtaaaattcaattggtatgtagatggggtggaagtgcacaatgccaaaacaaagccgcgagaagaacaatacaatagtacttaccgggttgtgagcgtgctcacggtgttgcaccaagactggctcaacggcaaggaatacaagtgcaaagtatctaataaagctctgcctgcgccgatagagaagaccatcagtaaggccaaagggcagccccgagagccgcaagtttacactcttcctccgagcagagatgaattgaccaagaaccaagtaagtttgacgtgcctggtgaagggcttctacccctcagacattgcggtggagtgggaaagtaatggtcaaccggaaaacaactacaagaccacgccgcccgtcctcgactccgatgggtctttctttctttattcaaagttgacagtagataagtcaaggtggcagcaaggtaacgtgtttagttgtagtgtaatgcacgaggccctgcataatcattatacccaaaagagtttgagcctctcaccaggaaaaggcggatcagacagctggcaggaggaggtaattaaattgtgtggacgggagttggtcagggcgcaaatagccatctgcggtaagagcacgggtggaggagagggtggaggggaaggtgggggagaaggcggcgggcagctctattctgcactcgccaacaagtgttgtcacgtcggatgcacaaagagatctcttgctcgattctgc

SEQ ID NO: 159 - DNA, 948 bp, synthetic construct
  source 1..948: mol_type = other DNA
atggagactgatactcttttgttgtgggtactgctcctgtgggttccaggctccacaggagacaaaacacacacctgtccgccttgcccggctcctgaagccgcgggtggccctagtgtgtttttgtttccgccgaaacctaaggataccctcatgataagccggacgcccgaggttacctgtgtcgtggtcgatgttagtcatgaggatccagaagtcaagtttaattggtacgtcgacggcgttgaagtccacaatgcaaaaactaaaccgcgagaagaacagtacaactccacctacagagttgtctcagttttgacagttctccatcaggattggctcaatggaaaggaatataagtgcaaggtcagcaataaagcgcttgccgcccctatagagaagaccattagcaaggcgaaaggacagccccgcgagccccaggtctatacgctgcctcctagcagagatgagctcacgaaaaatcaggtcagcttgacatgcttggtgaagggcttctaccccagtgacatcgcagttgaatgggagagcaacggccaacctgagaacaactacaaaacaacgcccccggttcttgacagcgatgggtccttctttctttactctaagcttacagttgataaaagcaggtggcagcaggggaatgtgttctcatgttccgtactgcatgaggctctgcattctcactacacccaaaaaagccttagcctgagccccggtaagggaggtagtgactcatggcaagaggaagtgattaagctctgcggccgggagttggtgagagcccaaatcgccatttgcggtaaaagtaccggagggggcgagggaggaggcgaaggtggaggtgaaggaggtggacagttgtactcagctcttgcaaataaatgttgtcatgttggttgcacgaaaagatctcttgcgaggttctgt

SEQ ID NO: 160 - DNA, 945 bp, synthetic construct
  source 1..945: mol_type = other DNA
atggagactgacactcttttgttgtgggtgcttcttctgtgggtacctggatccactggggataagacgcatacttgtccaccgtgccccgcaccggaagcggctggtggtccatcagtttttctgttcccaccgaaacctaaggacacgttgatgatatcacggacaccagaggttacgtgcgtagtggtggatgtgagccacgaggatccagaagttaaatttaattggtacgtagatggagtggaggttcataatgcgaagacaaagcctcgcgaggaacagtataattccacctatcgcgtcgtatctgtgcttacggtacttcaccaagactggttgaacggtaaggaatataaatgcaaggtttccaataaagcacttcctgcgccaattgagaagacaatatccaaagctaaaggtcaacccagggaaccgcaagtctacactctccccccgtctcgcgatgaattgacgaagaaccaggttagtctcacctgcctggtcaaggggttttacccctctgacatagctgtagaatgggagtctaatggacagccagagaacaattacaaaacgacccccccggtcctcgattctgatgggagtttttttctttattcaaaattgactgtcgataagtcaagatggcaacagggtaacgtattttcttgcagtgttatgcatgaagcattgcacaaccactatacacaaaaatcattgagtttgagtcccggtaaagggggaagcgactcatggatggaagaagtaatcaagctgtgcgggcgagagcttgtgcgagctcagatagcaatctgtggtaagtctacaggtggagagggtggcggtgaagaaggcgggggagagggaggccagctttattctgccctggctaacaagtgctgtcacgttggatgcacgaagcgctccctggcccgattctgc

SEQ ID NO: 161 - DNA, 945 bp, synthetic construct
  source 1..945: mol_type = other DNA
atggaaaccgatacattgcttttgtgggtcctccttctttgggttcctggctctacaggcgataagacgcatacttgtcccccatgtcccgctccggaagccgctggcggcccctccgtttttctgttcccgccgaaaccgaaagacaccctgatgatatcacgcactcccgaggtcacttgcgtggtagtcgatgttagtcatgaagatcctgaggtcaaattcaattggtatgtagatggcgttgaggtacacaacgcgaagacaaaaccccgagaagaacagtataactcaacctaccgcgtagtttcagttcttaccgtactgcaccaagactggttgaacggtaaagagtacaaatgtaaagtcagcaataaagctttgccagcacctatcgaaaaaaccatcagtaaggccaagggtcaacccagggagccgcaagtgtacactcttccccctagcagggatgaattgaccaagaatcaggtctctttgacgtgcctcgttaagggtttctatcccagcgatatagccgtagaatgggagtctaacggtcagccagaaaataactataagacaaccccgcctgttttggattccgacggctctttttttctctactctaagttgaccgttgataagagcagatggcagcagggaaacgtattttcttgttccgtgatgcacgaagccctgcacaatcactatacgcaaaagtctctgagcttgagtccgggtaaaggcggttctgactcctggcaggaggaagtcataaaactctgcggaagagagctcgtaagggcgcaaatcgctatttgtggtaagagcaccggtggggaaggaggcggtgaagagggtggcggcgagggtgggcaattgtattccgcgcttgccaataaatgttgtcacgtaggctgcacaaagcgaagtctcgctaggttctgc

SEQ ID NO: 162 - DNA, 942 bp, synthetic construct
  source 1..942: mol_type = other DNA
atggaaaccgacaccttgcttttgtgggtgctcttgctgtgggttccggggagcactggcgacaagacccacacatgtcccccgtgtccggcaccagaagcagcggggggaccgtcagtattcttgtttccaccgaagcccaaagacacattgatgatttcacgaactcctgaagttacctgtgtggttgtagatgtatcacacgaagacccagaagtcaaattcaattggtatgtcgacggggttgaagttcacaatgcgaagacgaagccccgggaggaacagtacaacagcacgtacagggttgtgagcgttcttactgtattgcaccaggattggctcaacggcaaggagtataaatgtaaagtttctaataaggctcttcctgccccaattgaaaagacgatatctaaagcgaagggccaaccacgggaacctcaggtgtacacacttccgcctagcagggatgagttgaccaagaatcaagtctctttgacgtgcctggtcaaggggttttacccatcagatatcgccgtcgaatgggagtcaaacggacaacccgaaaataactataaaactactccaccagttctggatagcgacggctcattttttctgtattcaaagctcactgtagacaagtctaggtggcagcagggtaatgtcttctcctgctcagtaatgcatgaggctcttcacaaccactatactcaaaagagcctttccctgtcacctggcggtggaagcgactcatggatggaggaggtaataaagctctgcggaagagaactggtacgcgcacaaatcgcaatttgtggtaagagtactggcggggaaggaggtggggaagaagggggcggtgagggcggacagctctattctgcacttgcaaacaaatgttgccacgtgggatgtactaagcgaagccttgcaagattctgc

SEQ ID NO: 163 - DNA, 942 bp, synthetic construct
  source 1..942: mol_type = other DNA
atggagaccgacacactgttgctgtgggtactcctcctgtgggtgccaggaagcacgggcgataaaacccacacatgccctccatgccctgctccagaggccgccggtgggccatcagttttcttgtttccgcctaaaccaaaggacacgcttatgatctccaggacccccgaagttacgtgtgtggtggttgatgttagtcacgaggacccggaagtcaagttcaactggtacgttgatggtgtagaggtgcacaatgcaaagacgaagccacgcgaagaacaatacaacagcacatatcgagttgtgagcgtactcacggtactgcatcaggactggctgaacggtaaagaatacaaatgtaaagtctccaataaggcacttcctgcgccgatagaaaaaacgatcagtaaggccaagggccaaccccgagaaccacaggtatatacgctcccaccgtcacgagacgagttgacaaaaaatcaggtctccctgacttgcctcgtgaaaggtttttatccctcagatattgctgttgagtgggaaagcaatgggcagccagagaataattataagacgactcctccggttttggattccgacggtagttttttcttgtatagtaagcttactgtagacaagtcaagatggcaacaaggtaatgtgttctcttgctcagttatgcatgaagctcttcataaccattacacgcaaaagagtctcagtctgagccccggtggcggtagcgacagttggcaggaagaggtgattaagttgtgcggtcgcgagctcgttcgggcccaaattgcaatctgcggaaaatctacgggcggagagggcgggggtgaggagggtgggggtgaaggtgggcagctctatagcgcccttgcgaataaatgttgtcacgtcggatgcacaaagaggtccctcgccaggttctgc

SEQ ID NO: 164 - DNA, 945 bp, synthetic construct
  source 1..945: mol_type = other DNA
atggaaactgacacactgttgctgtgggtgctgctcctttgggtacccggatcaaccggggataagacccacacttgccccccttgccctgcccccgaagcggccggaggtccttcagtatttttgtttccaccgaaacccaaagatactttgatgatatcaagaactcctgaagtcacctgcgtggtagttgacgtatctcatgaggatcccgaggtgaagttcaattggtacgtcgatggcgtcgaggttcataacgctaagactaagccgagggaagagcaatataattccacttatagggtggtgtccgtcttgactgttttgcaccaggattggttgaacgggaaagagtacaaatgtaaggtgagtaataaagctttggctgctcccatcgaaaagacaataagcaaggccaaggggcaacctcgggagccgcaggtgtacacccttcctcccagtagagacgaactgacaaaaaaccaggtgtccctgacctgccttgtgaaggggttttacccgagcgacatagcggttgaatgggagagcaacgggcaacccgagaacaactacaaaactacaccgcctgtcctggactccgatggaagcttcttcctctactccaaactgaccgtggacaaaagcagatggcaacaaggaaacgtattctcatgctcagtaatgcacgaagcattgcacaatcactacacccaaaagtccctctcactctcccctggtaagggcggatcagactcatggcaagaggaggtaattaagttgtgcgggagggagctcgtccgcgcgcaaatagccatttgtggcaagtccactggaggaggcgagggtggaggagagggtggtggggagggcaggcaactctacagtgcgctcgccaataaatgctgccatgttgggtgcacgaagcgcagtctcgcacaattctgc

SEQ ID NO: 165 - DNA, 945 bp, synthetic construct
  source 1..945: mol_type = other DNA
atggagaccgacactctgctgctctgggtactcttgctgtgggtgcctgggtctactggggataagacccacacgtgtcctccatgtccggcaccggaggctgctggcgggccttctgtattcctcttcccacccaagccaaaagacacattgatgatatcaaggacgccggaagtcacctgtgttgttgtggacgtttcccatgaagacccagaggtaaaattcaattggtatgtggacggcgtagaggttcacaacgccaaaaccaaaccccgagaggaacagtataatagcacatatcgagtagtatctgttctcacagtgctccatcaagactggcttaatggtaaagagtataaatgcaaagtttccaataaagccctcgctgcaccgatcgagaagacaatcagtaaagcgaagggccagcctcgggaaccgcaggtgtatactcttccaccctcaagagacgagctcactaaaaaccaagtttcattgacatgcctcgtcaaaggtttctacccatcagacatcgcggtcgaatgggaaagtaatgggcagccggaaaacaactataaaacgacgccgcccgtcttggattctgatggttcattttttctttactctaaattgaccgtcgataaaagtaggtggcaacaaggaaatgttttttcctgctccgtcctgcatgaagcgttgcacagtcactatacccagaagagtctttctttgtcacccggaaaaggcggttcagattcatggcaggaagaagtaattaaactctgtggccgcgagcttgttagggcgcagatagccatatgtggtaaaagcaccggaggaggtgaaggcggaggcgaaggaggtggggaaggaagacaattgtattctgcacttgcaaataaatgctgtcatgtggggtgcacgaaacgcagtcttgcacaattttgt

SEQ ID NO: 166 - DNA, 942 bp, synthetic construct
  source 1..942: mol_type = other DNA
atggaaaccgatacgctgcttctttgggttcttctcctctgggttccagggtccaccggcgacaaaacccatacctgccccccttgccctgcaccagaagcggcgggaggacctagcgtttttctttttcctccgaaaccgaaagataccctcatgatatcaagaacacctgaggttacttgcgttgtcgtggacgtgagtcacgaagaccccgaggtgaagttcaactggtatgtagatggagtggaggtccataatgcaaaaacgaaaccgagagaagaacaatacaactctacatatcgagtcgtgtcagtactcacggttttgcatcaagattggctgaacggtaaggagtacaagtgtaaggttagcaacaaggctctcgcggcgccgatagaaaagactataagtaaagcaaaaggccagcccagagaacctcaagtttacactctgcctcccagcagagatgaactgactaaaaatcaggtttcattgacctgtctcgtcaagggtttttatccaagcgacatagcagttgaatgggaaagcaacggtcaaccagaaaataattacaaaaccactccaccagtcttggactctgacggatccttctttctctattcaaaattgacggtggataaatctaggtggcagcaaggcaacgtcttctcttgtagcgttatgcatgaggcgctgcacaaccactacacacaaaagtctcttagtttgagcccgggcggcggaagcgactcttggcaagaggaagtgataaaactctgtggtcgagaattggtacgcgcgcagatcgctatctgcggcaagtccacagggggaggggaaggtggcggggaaggtggtggcgagggcaggcagttgtatagtgcacttgccaacaagtgctgccatgtggggtgcaccaagcgcagtttggcacggttctgc

SEQ ID NO: 167 - DNA, 942 bp, synthetic construct
  source 1..942: mol_type = other DNA
atggaaacggacacccttctgctctgggtactgctgctctgggttcctggttctaccggtgataaaactcacacttgtcccccgtgtccggcaccagaagccgcaggagggccatctgtctttctttttcccccaaaacccaaggatacactgatgatctcccgcactcccgaagttacttgtgtcgtagtagacgtttctcacgaggacccagaggtgaaattcaattggtatgttgacggagtagaggtgcataatgccaagacaaagccccgagaggaacaatacaattcaacctacagagtagtgtccgttcttacggttctccatcaggattggctcaacggtaaggaatataagtgcaaggtaagcaacaaagcgctggccgcacccattgagaaaaccatttcaaaagctaaaggccaaccccgcgaaccacaagtttatactctccccccaagtcgcgatgaacttacaaaaaatcaagtctcattgacgtgcttggtcaaaggcttctacccgagcgatatcgctgttgaatgggagtctaatggacaaccggaaaataactataaaactacacccccagtcctcgattcagacggcagcttcttcctgtattcaaaactgacggttgacaaatcacgctggcaacagggtaacgttttttcctgtagcgttcttcatgaagccttgcacagtcactacacccagaagtcccttagcttgtcacctggcgggggttcagactcttggcaggaggaggtaatcaaactgtgcggaagagaactggtgagggctcagattgcaatttgtgggaagagcacgggtggcggtgaaggaggtggcgagggcggaggagaggggaggcaactctacagtgcgttggctaataaatgctgtcacgtcggctgtactaagagaagcctcgccagattttgc

SEQ ID NO: 168 - DNA, 942 bp, synthetic construct
  source 1..942: mol_type = other DNA
atggaaacagatactttgttgctgtgggtactcctcctctgggtacctgggagcaccggggacaagacgcatacttgccctccgtgccctgcaccagaagccgctggtggcccatctgtgtttttgttcccccctaagccaaaagacacattgatgatttcacgaactccagaagtgacttgcgtagttgttgacgtatcacacgaagaccccgaggttaaatttaattggtatgtggacggggtcgaggtgcataacgccaaaaccaaaccccgggaggaacaatataactctacgtatcgggtcgtatctgtgttgaccgtccttcaccaagattggttgaacggcaaggaatataagtgtaaagtgtctaataaagcattggctgccccgatagaaaagacgatctctaaagccaagggccaacccagagagcctcaagtatatactctcccaccgagtcgagatgagctcactaagaaccaggtgtcactcacgtgtctggttaaaggattttaccctagtgatatagccgtcgagtgggaatcaaatgggcagccggagaataactataagaccacgcctccagttctcgattccgatggtagctttttcctttactctaaacttacggtcgacaagtccaggtggcaacagggcaatgtattttcttgctccgtcatgcacgaggctttgcacaaccattacacgcaaaagtcactgtccctgtctcctggaggcggttctgacagttggcaggaggaggtaatcaaattgtgtgggcgggagttggttagggcgcaaattgctatttgcggcaaaagtactgggggcggtgaaggcggaggcgagggaggaggagaaggtcgacaactgtattctgccttggcgaacaaatgctgtcacgtcggctgtacgaaacggtctttggcccagttttgt

SEQ ID NO: 169 - DNA, 942 bp, synthetic construct
  source 1..942: mol_type = other DNA
atggaaactgacactcttctgttgtgggtccttctgctgtgggttcctggctctactggagataagacacacacttgtccgccatgccctgcgccggaagcggcgggaggaccgtccgttttcctgttccctcccaaacccaaagacacgttgatgattagtcgcacgccagaagttacgtgcgttgtcgtagatgtatcccacgaagaccccgaggtgaagttcaattggtatgtagatggggtggaggtccataacgctaagaccaaaccacgcgaggaacaatataattctacgtaccgcgtagtgagcgttctcacagttcttcaccaggattggcttaacggcaaggagtataagtgtaaggtgtctaataaggccttggctgccccgatcgaaaaaacgataagtaaagcaaagggtcaacctagagaaccccaagtgtacactctcccgccatcacgggatgaattgactaagaaccaagtgtcactcacgtgtcttgtaaagggcttctacccatccgatatagccgttgagtgggaatccaatggtcagccagagaacaactataagacaactccgcccgtacttgatagtgacggttcctttttcctttacagtaaattgacggtagataagtctcgctggcagcaaggaaacgtcttttcttgttcagtgcttcatgaggcgcttcactcacactatactcagaagagtttgagtttgtctccaggtggaggcagcgactcatggcaagaggaagtaatcaaactgtgtggtcgcgaattggtacgagcacagatcgcgatctgcgggaaatcaacaggtggcggcgaaggcggcggggaaggcggcggcgaaggtaggcaactttactcagcccttgcgaacaaatgttgccacgtaggctgtactaagagaagtctcgcccagttttgc

SEQ ID NO: 170 - DNA, 945 bp, synthetic construct
  source 1..945: mol_type = other DNA
atggagacagatacccttctgttgtgggtccttctgctttgggtgccgggaagtacaggcgacaagactcatacctgccccccttgtccagcaccagaagcagctggcgggccaagcgtgttcctgtttccacctaagcccaaagatacgttgatgatcagccgcaccccggaagtaacctgtgtagtagtagatgtgtcccacgaagaccccgaagtaaagtttaattggtacgtcgatggtgtcgaagtacataacgctaaaacgaagccccgagaagagcagtacaacagtacttacagagtagtttctgttcttacagtgctgcatcaggattggctgaacgggaaggagtataaatgtaaagtctcaaacaaggcacttgcggcaccaatagagaagacaatatctaaggccaaagggcagcctagagagccacaagtatatacgctgccccccagcagggacgagctgacaaagaaccaagtgtcactgacctgccttgttaagggcttctatccgagtgatattgctgttgaatgggaaagtaacggacagccggagaacaactataaaactactccacccgtgttggatagtgacggtagcttttttctgtactccaagttgacggtagacaaaagtcggtggcagcaggggaacgtattttcttgttctgtcatgcacgaagctcttcacaatcactatacgcagaagtccctctctctctctcctgggaagggtggttccgacagctggcaggaggaggtcattaaactgtgtggtagagagctggtacgggctcaaattgcaatttgtggtaagagtactggcggtggcgaggaagggggtggggaggagggcggaggtaggcagctctactctgctctcgccaacaagtgttgtcacgtcgggtgtactaaaagatcacttgcccgcttttgt

SEQ ID NO: 171 - DNA, 945 bp, synthetic construct
  source 1..945: mol_type = other DNA
atggaaaccgataccctgctcttgtgggtcctcctgctttgggtcccaggttccacaggcgacaaaacacatacatgcccgccgtgtccggcgcctgaagcagcaggaggccccagtgtattccttttccctccaaagccaaaagatacgttgatgatatctaggacacctgaggttacctgcgtcgtagtggacgtatcccacgaagacccagaagtcaagtttaactggtatgtggacggagtggaggtacacaatgcaaagacaaagccgcgagaggaacaatataattccacctatagagtcgtgtcagtccttacggtcttgcaccaggactggctcaatggtaaggagtataagtgcaaagtatcaaacaaagctctcgcagcgcccatcgaaaagaccatcagcaaagctaagggccagccaagagagcctcaagtgtacacgttgccgccttcaagggacgagctcactaaaaatcaggtatcacttacgtgtcttgtcaaagggttttatccttccgacatcgcggttgaatgggagagcaatggacagccggagaataattataaaacgacgccgccggtccttgacagcgatggttcatttttcctttactcaaagctgacggttgataagtctaggtggcagcaggggaacgtcttttcctgtagtgtacttcatgaggcgctccattctcattacactcagaagtcactgagcctttcacccggcaaaggtggatcagactcctggcaagaagaggtaatcaaactctgtgggagggaactcgttcgagcccagattgcaatctgtgggaaaagcacaggcggaggggaagaagggggtggcgaagaaggtgggggcaggcagctctattcagctcttgccaacaaatgctgtcatgtaggctgcacaaagcgatcactggcgagattctgt

SEQ ID NO: 172 - DNA, 945 bp, synthetic construct
  source 1..945: mol_type = other DNA
atggaaaccgacaccctgctgctctgggttcttctcctctgggttcccggctcaaccggagataaaactcatacttgcccaccctgcccggctcccgaggcagcaggtggaccctcagtatttttgttccctccgaaacctaaagatacacttatgattagccggacccctgaggtaacgtgtgtggtggttgacgtaagtcatgaagatccagaagtaaagtttaactggtacgtagacggtgtggaggtacataatgcgaagacaaaaccacgagaggaacagtataactctacctaccgcgtagtaagcgtacttactgtgctccaccaagactggcttaacgggaaagagtataagtgtaaagtcagtaataaagcactggccgccccgatcgaaaaaacaatcagcaaggccaaaggacaaccaagggagcctcaggtctatactcttcccccgagtagggatgagcttaccaagaaccaggtgtctctgacatgccttgtcaagggattttacccgagtgacatagccgtagaatgggagtcaaacggccaacctgaaaacaactataagaccacgcctcccgtactcgactcagatggaagctttttcctctatagcaagctgaccgtcgacaaaagtaggtggcaacagggaaacgtctttagttgttccgtcatgcacgaagctttgcataaccattacacccagaagagtctttccctttcccctggcaaggggggctccgactcctggcaagaggaagtaatcaaactgtgtgggcgcgagcttgtccgcgcgcaaatagccatttgcggaaaaagtactggaggaggagaggaaggcggcggcgaggaaggtgggggcaggcagctgtacagtgccttggctaacaagtgctgccatgtcggctgtacgaaaaggtctcttgctcaattctgt

SEQ ID NO: 173 - DNA, 945 bp, synthetic construct
  source 1..945: mol_type = other DNA
atggaaactgatactcttctcctttgggtgctcctcctctgggttcccgggtccacaggcgataagacacatacctgtccaccctgcccagcacctgaagctgcaggcggccccagcgtattcctgtttcctccgaagccgaaagacacacttatgatttcccggacgcctgaggtaacttgcgtcgtagtagatgtgtctcacgaagaccccgaggtgaaattcaactggtacgttgatggtgtggaagttcataatgcgaaaactaaaccacgagaggagcaatataactcaacttatagagttgtgagcgtcttgacggtactgcaccaggactggctgaatggcaaagagtacaaatgcaaagtctcaaataaggcgttggcggctcccatagagaaaactatcagcaaagccaagggtcaacctcgggagccacaagtgtatactcttccgcctagtcgcgacgagctcacaaagaatcaggtgagtcttacttgtttggttaagggtttctaccccagtgacattgcggtcgagtgggaaagtaacggacagcctgaaaacaactataaaacaacgcctccagtactcgattcagatggttcattctttctttattccaaactcacagtcgacaagagtagatggcaacaagggaacgtgtttagctgtagcgtactccatgaggcactccactctcactatacccaaaagtctctcagcttgtcacccggaaaaggcggttctgacagttggcaagaggaagtgattaaattgtgtgggcgggaacttgtgagggctcaaatcgcgatttgcggcaagtccactggtggcggcgaggaaggaggaggtgaagaaggaggaggtaggcaactgtattcagcgttggcgaataaatgctgccatgttggatgtactaaacggagccttgctcagttctgc

SEQ ID NO: 174 - DNA, 942 bp, synthetic construct
  source 1..942: mol_type = other DNA
atggaaactgacaccttgttgctttgggtattgcttctgtgggttccgggtagcacgggtgataaaacgcatacttgccctccttgcccggcacctgaagctgccggaggtccttccgtgttcctgttcccacctaagccaaaagacacacttatgatttctcgcacaccagaagtaacgtgcgtcgtagttgacgtctcccatgaagacccggaggtaaaatttaattggtacgtcgacggggtagaagttcataacgcaaagactaaaccacgagaagagcaatacaactctacatacagagtagtaagcgttctcaccgttcttcatcaagattggctcaacggaaaggagtataagtgtaaggtgtccaataaagcgttggccgcaccaatcgaaaagaccataagcaaagccaaaggccaaccccgcgaaccgcaggtgtacacacttcccccgtccagggatgaattgacaaaaaaccaagtttccctcacgtgtctcgtcaagggattctacccgagtgatatcgcagttgaatgggaaagcaatggtcagcccgagaataactacaagactactccccctgtgttggactcagacggctcattcttcctctacagtaagttgactgtggacaaaagtcggtggcagcaaggcaatgtcttcagttgtagtgtaatgcatgaagcactccacaatcattacacccaaaaatccctgagcctgtccccgggcggaggttcagattcatggcaggaggaagttataaaactgtgcgggcgcgagttggtgagggcgcagatcgcaatctgtggaaagagtacgggaggtggcgaagagggtggtggagaagagggaggaggtcgacaactgtattccgcgctcgcgaacaagtgttgccacgttggctgcaccaaacgaagcctggctcgattttgc

SEQ ID NO: 175 - DNA, 942 bp, synthetic construct
  source 1..942: mol_type = other DNA
atggagactgacacccttctcctctgggtcctcttgctttgggtccctggctctactggtgacaagacacacacttgtccaccttgcccggctcccgaggcggcaggaggaccaagcgtttttctgttccctcccaaaccaaaggatacgcttatgatctctcgaacgccggaagttacttgcgtagtagttgatgtctcccatgaagatcccgaagtgaagttcaactggtatgtagatggtgtggaagttcataacgcgaaaaccaaaccacgcgaagaacagtataacagtacttatcgggttgtttcagtactcacggtgctccatcaagactggcttaatggaaaggagtataaatgtaaggtaagtaacaaggcattggcggctcccatcgagaagacaatctccaaagcaaaagggcaaccacgggagcctcaggtgtatacgttgccgcccagcagagatgaacttactaagaatcaggtgagtctcacttgtctcgtcaagggcttctatcccagcgatatagccgtagaatgggagagtaacggtcagccggagaacaactacaaaacaaccccgcctgttttggactccgatgggagtttttttctctacagcaaactcacggtagacaaaagcaggtggcagcagggcaatgttttcagttgctctgttctccacgaagccctccactcccactatactcagaagtctctgagtctctcaccagggggaggtagcgatagctggcaggaggaagtgatcaagttgtgcgggcgcgaactcgtgcgggcacaaattgctatatgcggtaaaagtacgggaggtggagaggagggtggaggtgaagaaggcggtggtagacaattgtatagtgcgctcgccaacaagtgttgtcatgtcgggtgtacgaaacggtccttggcgcggttttgc

SEQ ID NO: 176 - DNA, 942 bp, synthetic construct
  source 1..942: mol_type = other DNA
atggaaactgacacacttcttctgtgggtactcttgttgtgggttccgggctcaacgggtgacaagacacatacttgtccaccatgtcccgccccagaagctgcgggaggaccatcagtttttttgttccccccgaaaccgaaggataccctcatgataagtcgaacgcccgaagtcacttgcgtggtggttgatgttagccacgaggacccagaagtgaagttcaactggtacgtggacggggtcgaagttcataatgcgaaaacaaagcctcgcgaggaacagtacaactctacatacagggttgtgtctgttttgacagtcttgcaccaagattggctcaacgggaaggaatataagtgtaaggtaagcaataaagcactggcggccccgatcgaaaaaacgatatccaaggccaagggccagccccgagagcctcaggtatatactctgccgccaagccgggatgaactgactaaaaaccaggtctctttgacttgtcttgtcaagggattttacccaagtgacattgcggtagagtgggaaagcaacggtcaaccagaaaacaattacaagacgacaccgccggtactcgactcagatggatcctttttcctgtatagcaagctgacagtggacaagtcccggtggcagcaagggaacgtattttcatgcagcgtgatgcatgaggctcttcacaaccattacacacagaaaagtctgtcattgagccctggcggcgggagcgattcttggcaagaagaagttataaaactttgcggtcgagagctggttcgggcacaaattgctatctgcggaaaatctacaggaggaggcgaggagggagggggcgaagaaggcggggggagacagttgtacagtgcgctcgctaacaagtgttgccacgtcggttgcacaaagagatccctggctcaattctgt

SEQ ID NO: 177 - DNA, 942 bp, synthetic construct
  source 1..942: mol_type = other DNA
atggagacagatactctcttgctgtgggtgctgctcttgtgggttcctggaagtaccggtgataaaactcacacctgtcccccgtgtcccgcaccagaagcggccggtggtccctccgtttttctcttccctcctaaacctaaggacacacttatgattagcagaactccagaagttacgtgcgtagtcgttgacgttagtcatgaagatcctgaggttaagttcaactggtacgtagacggagtagaggtccacaacgccaagacgaaaccccgagaagagcagtataattctacctatcgagttgtttcagtattgacggtgcttcaccaagattggctgaatggcaaagagtataagtgcaaggtaagcaacaaagcactcgcggctcctatcgagaaaactatttccaaagctaagggccagcctcgcgaaccacaagtctataccctgccaccgagtcgggacgaactcaccaagaaccaagtgtctcttacttgcctcgttaaaggtttttatcccagcgacatagccgtcgaatgggagtccaatggccaacctgagaacaactataaaactacccctcctgtacttgatagcgacggaagttttttcctctattcaaaactcacagttgataagtctcgatggcaacagggcaacgtcttctcttgcagtgtgttgcatgaagctctgcactctcattacacacagaagagtttgtctctcagtccaggtggcggctcagatagctggcaggaagaagtaatcaagttgtgcggcagggaactggtaagggcacagatagccatttgtggaaaatctacgggtggcggtgaggaaggcggcggagaagaagggggaggtcggcagctgtatagtgcactcgcaaacaagtgctgccatgtcgggtgcaccaagcgatcccttgcccagttttgc

SEQ ID NO: 178 - DNA, 945 bp, synthetic construct
  source 1..945: mol_type = other DNA
atggaaactgatacgctcctcctttgggttcttctcctctgggtacccggaagcactggtgacaaaacgcacacctgtccaccgtgtcctgctccagaggcggccgggggaccgtccgttttcctttttcctcccaaacctaaggatacccttatgatctctcgcacgcccgaggttacctgtgttgtggttgacgtgtcccatgaagacccggaagtaaaatttaattggtacgtggacggggtcgaggttcataacgcaaagaccaagccacgagaggagcaatataattccacctatcgcgtagtctccgtcctcaccgtgcttcaccaggattggctcaacgggaaggaatacaaatgtaaagtcagtaataaggctttggcggccccgattgagaagactataagtaaggctaagggacagccacgagaaccgcaagtttatacattgcccccctctagggatgagttgactaagaatcaggtgtcactcacttgtctggtaaaagggttctacccgtccgacatcgctgtggaatgggaaagcaatgggcaacctgaaaataattataagacaacccctccggtgcttgatagcgacggatcattctttctctattccaagcttactgtagataagagtcgatggcaacaggggaacgtattcagttgctctgttctccatgaggccctgcatagtcactacacccaaaaaagccttagtttgagtcccgggaaaggaggctccgattcttggcaagaagaggtaataaagctgtgtggacgagaacttgtccgagcacaaattgcgatttgtggcaaatctacaggagggggagaaggaggcggcgaagggggaggcgagggcaggcaggattattccgctctggcgaacaaatgttgccatgttggatgcacgaaacgaagcctggctcagttttgc

SEQ ID NO: 179 - DNA, 942 bp, synthetic construct
  source 1..942: mol_type = other DNA
atggaaaccgacaccctcctcctttgggtacttcttctctgggttccgggtagtacaggagataaaacgcatacctgcccaccgtgtcctgcacctgaggccgctggaggaccttccgtcttcctctttccacccaagccgaaagacacactcatgattagtagaactccagaggtcacgtgtgttgtggtggacgtcagtcacgaggaccctgaggttaagttcaactggtacgttgatggcgtagaagtccacaatgcaaagaccaaaccgagagaggagcaatataacagtacatatagggttgttagcgtacttactgttttgcatcaagactggttgaatgggaaggaatataaatgtaaagtctccaacaaggctctggctgcaccaatagaaaaaactatttctaaggcaaagggtcagcctagagagcctcaagtctataccttgccaccgtcaagagacgagctcactaaaaatcaggtgagcctgacctgtcttgtgaagggcttttacccgtcagatattgccgtggagtgggaatcaaacggtcagccggagaataactacaagacgaccccaccagtactcgatagcgatgggtctttctttctgtactccaagctcaccgtggacaaatcacgctggcaacagggcaacgtctttagttgcagcgtactgcacgaggcactgcacagccactacacacaaaagagtctttctctgtctcccggtggtggctccgatagttggcaggaagaagtcataaagctttgtggaagagagcttgtacgagcgcagattgcaatctgcgggaagagcactggaggaggtgagggagggggtgagggcgggggcgaaggacgccaggactattcagcacttgcaaacaaatgctgccatgtagggtgtacgaagcgctcactggcccggttttgc

SEQ ID NO: 180 - DNA, 942 bp, synthetic construct
  source 1..942: mol_type = other DNA
atggagaccgacactttgctgctttgggtgttgctgctgtgggtccccggtagtacgggagataaaacacatacctgccccccatgcccagcccccgaagctgcaggggggccctctgttttcctttttccacccaaacctaaagatactctgatgattagtcggactccggaagtgacttgcgtcgttgtcgacgtctctcatgaggatccagaagttaagtttaactggtatgtcgacggggttgaggttcataatgcaaaaactaaaccgagagaagaacagtacaactctacttatagggttgtcagtgtactgaccgtcctgcaccaggattggcttaacggtaaggagtataagtgtaaagtgtccaataaagcccttgccgcacccatcgagaaaaccatctccaaggcaaaaggacagccaagggaaccgcaggtatatacacttccgccaagccgagacgaacttacgaagaaccaggtgtctctcacgtgtctcgtaaaagggttttatcccagcgatatcgcagttgagtgggagagcaatgggcagccagagaataattataagacaacccctcccgtgctggattcagacgggagtttttttctttactctaagctgaccgtagacaaaagtcgatggcagcaaggcaacgtcttttcctgctccgttctccatgaagcactgcatagccattatacccagaagtcactgagcctctctccagggggcgggtccgattcatggcaggaagaggtaatcaaactctgtggacgcgaactggttcgcgcgcagatagcgatttgcggcaaaagcacaggcggtggggaaggcggtggcgagggcggtggtgaaggtcgacaagattattctgctctcgctaacaagtgttgtcatgtaggatgtactaaaaggagtcttgcgcagttctgt

SEQ ID NO: 181 - DNA, 945 bp, synthetic construct
  source 1..945: mol_type = other DNA
atggagacggacactcttctcctgtgggttctcctcttgtgggttccaggatctaccggcgataagacgcacacatgcccaccctgtcctgcgcctgaagccgcggggggacccagcgtttttctcttcccgccgaaaccgaaagacacacttatgatcagccggactcccgaggttacctgcgtggtggtagatgtatctcacgaggatcccgaggtcaaattcaactggtacgttgatggggttgaagttcataatgccaaaacgaagccaagagaagagcagtataactccacatatagagttgtttccgtcttgactgttcttcaccaagattggctgaatgggaaggagtacaaatgtaaagttagcaacaaggcactcgccgctcccattgaaaaaactataagcaaagctaagggccaaccgcgcgaaccacaggtctacacgttgccgccctctagggacgaactcacgaagaatcaggtttcccttacctgcctcgttaaaggattctacccctctgacatagcggttgaatgggagagcaacggtcagcctgagaacaactacaaaacgacgcctccggtgttggattccgacggtagttttttcctctatagtaagctgacagtggataaatctcggtggcagcaagggaatgtattctcctgttcagtcctgcatgaagccctccactcccattatacacagaaatctctttctctgagtcccggtaaaggtgggagtgactcttggcaggaagaggtaattaagttgtgtggaagggagctggtaagagcacagattgccatctgtggcaaatccacgggcggcgaaggtgaggggggtgagggggaaggggggtccagacaactgtattctgctctggcgaataagtgttgccatgtagggtgcactaaacggtccttggcgcagttctgt

SEQ ID NO: 182 - DNA, 942 bp, synthetic construct
  source 1..942: mol_type = other DNA
atggagactgacacactgctcctctgggtccttttgctctgggttccggggtccaccggtgataaaactcatacgtgcccaccttgccccgcaccggaggctgctggaggaccctctgtcttcctgttcccgccgaagcctaaagacacattgatgatcagtcgaacaccggaagtcacctgtgtagtggttgatgtgagccatgaggaccctgaagtaaaatttaactggtatgttgatggcgtagaagtacacaacgcgaagactaaaccaagggaagagcaatacaactctacctatagggtcgttagcgtactgactgtgcttcaccaagactggcttaacgggaaggagtacaagtgcaaagtgagcaataaggccctcgccgcgcctatcgagaaaaccatttccaaagccaagggtcaaccaagggagcctcaggtttacaccctgcccccttcaagggatgagttgacaaaaaaccaggtaagtctgacgtgtctcgttaagggattctacccgtcagatatcgcggtagagtgggagagcaacggtcagccagaaaataattacaaaacaacacctccagttttggactctgatgggagtttttttctttattctaagttgacagtggataagtcacgctggcaacaggggaacgtatttagctgctcagtacttcatgaagcgttgcattctcactacacacagaagagcctctccttgagtcccggaggtggctctgattcttggcaggaggaggtaataaaactttgtggtagagaactggttcgcgctcagatagctatttgtggaaaatccactggcggtgaaggtgaaggtggagaaggagagggcggaagccggcagttgtactctgccctggctaataagtgctgtcacgtgggctgcactaagcggagcttggcaagattttgc

SEQ ID NO: 183 - DNA, 942 bp, synthetic construct
  source 1..942: mol_type = other DNA
atggaaaccgacacgctgctgctgtgggtgctgttgttgtgggttccaggctcaactggcgataaaactcatacctgtccaccttgtcctgcgcctgaggcagctggagggcctagcgtgttcctgttcccccccaaacccaaagacacgctcatgattagccgaacccctgaagtgacctgcgttgttgtggacgtaagccacgaagaccccgaagttaagtttaattggtacgtcgacggtgttgaggttcataacgcgaagactaagccgagagaggagcaatataacagcacctaccgcgtagtctcagttcttaccgtgctccaccaggactggcttaacgggaaggaatacaaatgcaaagtttccaacaaagccttggcagccccaatagagaagacaatatctaaggcgaaaggccaaccgcgggaaccgcaagtttataccctcccaccgagcagggatgagctgacaaaaaatcaggtttccctcacttgtctggtcaagggattttatccttcagacatagccgttgaatgggagagtaatgggcagccggagaataattacaagaccacccccccggtgttggacagcgacggttccttctttctctattctaaacttaccgtcgacaaatcacggtggcaacaaggaaatgtattctcatgcagtgtattgcacgaagctctgcactctcattacacccaaaaatccctctctctcagccctggcggtggatctgattcttggcaggaagaggtgattaaactgtgtgggcgagagcttgtccgagctcagatcgctatttgtggcaagagtaccggaggcgagggtgagggaggcgaaggcgagggcggaagccggcaactctatagcgcactcgctaataaatgttgtcatgtcggctgcacgaagcgctcactggcgcagttctgc

SEQ ID NO: 184 - DNA, 945 bp, synthetic construct
  source 1..945: mol_type = other DNA
atggagacggacacactgctcttgtgggtactgctcctttgggtgccaggaagtacaggagacaaaacgcatacctgtcctccatgccccgctcccgaggctgccggcggaccaagcgtatttctcttcccccctaaacctaaagacacattgatgataagtaggacgcctgaagtaacgtgtgttgtcgttgatgtaagccatgaagatcctgaagtaaagtttaattggtatgttgatggcgtagaagtacataacgctaagacgaagccacgggaagagcagtataactcaacttaccgcgttgtaagcgtgcttaccgtcctgcatcaggattggctgaatggtaaggaatataagtgcaaagtaagcaacaaagcattggccgcaccaatagagaagacgattagtaaagcaaaaggccagcccagagagccgcaggtttatacacttccaccaagcagagatgaacttacgaagaaccaggtgtctctgacttgtctggtcaagggtttctatccttccgacattgcagtggagtgggaaagcaatgggcagcccgaaaacaattataagacgacacctccagtgttggactcagacggttcctttttcttgtattccaaacttacagtggataagtcaaggtggcagcaaggcaacgtattttcttgtagtgttttgcacgaagccctgcattcccactatactcaaaagagcctcagtctgtccccaggaaagggagggagtgacagttggcaagaggaggtaataaaattgtgtggcagagagcttgtgcgcgctcagatcgcaatatgcgggaaatctactgggggtgagggtgagggcggcgagggagaggggggcagtcgccaagattattccgcccttgcgaataagtgttgtcacgtcggatgtactaagagatcattggctcagttttgt

SEQ ID NO: 185 - DNA, 36 bp, synthetic construct
  source 1..36: mol_type = other DNA
acgggaccgatccagcctccggactctagagccacc

SEQ ID NO: 186 - DNA, 36 bp, synthetic construct
  source 1..36: mol_type = other DNA
tgataaaccggttagtaatgagtttgatatctcgac

SEQ ID NO: 187 - protein, 29 aa, synthetic construct
DSWKEEVIKLCGRELVRAQIAICGKSTAS

SEQ ID NO: 188 - protein, 29 aa, synthetic construct
DSWKEEVIKLCGRELVRAQIAICGKSTWS

SEQ ID NO: 189 - protein, 29 aa, synthetic construct
DSWMEEVIKLCGRELVRAQIAICGKSTAS

SEQ ID NO: 190 - protein, 29 aa, synthetic construct
DSWMEEVIKLCGRELVRAQIAICGKSTWS

SEQ ID NO: 191 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTASDAAGANANAGARQLYSALANKCCHVGCTKRSLAQFC

SEQ ID NO: 192 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSDSWKEEVIKLCGRELVRAQIAICGKSTASDAAGANANAGARQLYSALANKCCHVGCTKRSLAEFC

SEQ ID NO: 193 - skipped

SEQ ID NO: 194 - protein, 20 aa, synthetic construct
METDTLLLWVLLLWVPGSTG

SEQ ID NO: 195-199 - skipped (5 entries)

SEQ ID NO: 200 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 201 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 202 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 203 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPG

SEQ ID NO: 204 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGRQLYSALANKCCHVGCTKRSLARFC

SEQ ID NO: 205 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGGGQLYSALANKCCHVGCTKRSLARFC

SEQ ID NO: 206 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGRQLYSALANKCCHVGCTKRSLARFC

SEQ ID NO: 207 - protein, 296 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGGGQLYSALANKCCHVGCTKRSLARFC

SEQ ID NO: 208 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGEGGGEEGGGEGGQLYSALANKCCHVGCTKRSLARFC

SEQ ID NO: 209 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGGGEEGGGEGGQLYSALANKCCHVGCTKRSLARFC

SEQ ID NO: 210 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWMEEVIKLCGRELVRAQIAICGKSTGGEGGGEEGGGEGGQLYSALANKCCHVGCTKRSLARFC

SEQ ID NO: 211 - protein, 294 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGGGEEGGGEGGQLYSALANKCCHVGCTKRSLARFC

SEQ ID NO: 212 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAQFC

SEQ ID NO: 213 - protein, 295 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAEFC

SEQ ID NO: 214 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGRQLYSALANKCCHVGCTKRSLARFC

SEQ ID NO: 215 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTGGGEGGGEGGGEGGGQLYSALANKCCHVGCTKRSLARFC

SEQ ID NO: 216 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGRQLYSALANKCCHVGCTKRSLARFC

SEQ ID NO: 217 - protein, 316 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGGEGGGEGGGEGGGQLYSALANKCCHVGCTKRSLARFC

SEQ ID NO: 218 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWMEEVIKLCGRELVRAQIAICGKSTGGEGGGEEGGGEGGQLYSALANKCCHVGCTKRSLARFC

SEQ ID NO: 219 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWQEEVIKLCGRELVRAQIAICGKSTGGEGGGEEGGGEGGQLYSALANKCCHVGCTKRSLARFC

SEQ ID NO: 220 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWMEEVIKLCGRELVRAQIAICGKSTGGEGGGEEGGGEGGQLYSALANKCCHVGCTKRSLARFC

SEQ ID NO: 221 - protein, 314 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGGGSDSWQEEVIKLCGRELVRAQIAICGKSTGGEGGGEEGGGEGGQLYSALANKCCHVGCTKRSLARFC

SEQ ID NO: 222 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAQFC

SEQ ID NO: 223 - protein, 315 aa, synthetic construct
METDTLLLWVLLLWVPGSTGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGKGGSDSWKEEVIKLCGRELVRAQIAICGKSTASDAAGANANAGARQLYSALANKCCHVGCTKRSLAEFC

SEQ ID NO: 224 - protein, 309 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLYSALANKCCHVGCTKRSLARFCGGGGSGGGGSGGGGSSWMEEVIKLCGRELVRAQIAICGMSTWS

SEQ ID NO: 225 - protein, 329 aa, synthetic construct
METDTLLLWVLLLWVPGSTGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSQLYSALANKCCHVGCTKRSLARFCGGGGSGGGGSGGGGSSWMEEVIKLCGRELVRAQIAICGMSTWS